(19) United States
(12) Patent Application Publication
(10) Pub. No.: US 2020/0121598 A1
(43) Pub. Date: Apr. 23, 2020

(54) PROGRAMMABLE THERMORESPONSIVE GELS

(71) Applicant: City of Hope, Duarte, CA (US)

(72) Inventors: Abraham J. Domb, Duarte, CA (US); Jacob Berlin, Duarte, CA (US)

(21) Appl. No.: 16/622,656

(22) PCT Filed: Jun. 29, 2018
(86) PCT No.: PCT/US2018/040319

§ 371 Date: Dec. 13, 2019

Related U.S. Application Data
(60) Provisional application No. 62/526,630, filed on Jun. 29, 2017.

(51) Int. Cl. A61K 9/06; A61K 9/00; A61L 29/06

(52) U.S. Cl. CPC A61K 9/06; B33Y 70/00; A61L 29/06; A61K 9/0019

(57) ABSTRACT

Provided herein, inter alia, are non-crosslinked polymers that possess thermoresponsive properties. These polymers possess a cleavable bond that breaks under certain conditions The disclosure also provides pharmaceutical compositions containing the polymers and therapeutic agents, methods for delivering the therapeutic agents, and kits, syringes, and catheters containing the polymer compositions and therapeutic agents.

### CROSS-REFERENCE TO RELATED APPLICATIONS

This application claims priority to U.S. application Ser. No. 62/526,630 filed Jun. 29, 2017, the disclosure of which is incorporated by reference herein in its entirety.

### BACKGROUND

The costs associated with drug development are enormus. Improving the effectiveness of existing drug therapies has led to the design of new materials for drug delivery. For example, cell-based therapies have attracted growing interests for the treatment of cancers such as glioblastoma multiforme and muscle-invasive bladder cancer which are highly invasive cancers that are impossible to completely eliminate with surgery and/or radiation. However, using engineered cells to treat these types of disorders is still hampered by a lack of an effective route and method of delivery. In addition, currently available delivery systems cannot achieve targeted and long-term delivery while at the same time responding to environmental changes (such as temperature, pH, etc.) which are caused by many disorders in organs and blood vessels.

Biodegradable polymers play an important role in drug delivery. Because these polymers degrade after a certain period of time, sustained drug release can be enhanced and surgical removal after drug depletion can be avoided. However, many biodegradable polymers have disadvantages in requiring organic solvents for drug loading thereby limiting the selection of drugs that are not adversely affected, i.e. denaturation of protein drugs, by such solvents. Biodegradable polymers also suffer from inconsistent drug release kinetics and lack of response to physiological changes in living organisms. Bioresponsive polymers are another class of polymers widely studied, especially as devices for the delivery of physiological unstable agents, such as protein drugs including growth factors. This class of materials is responsive to physical, chemical, or biological stimuli. However, bioresponsive polymers have problems in non-biodegradability and non-sustained drug release.

Polymer systems that undergo phase transitions in response to environmental stimuli such as temperature and pH have been widely investigated for drug delivery, separations, and diagnostics applications. In particular, thermoresponsive gels have been suggested for potential use as drug carriers and cell delivery scaffolds. However, to date, there have been no reports of non-crosslinked thermoresponsive polymer gels that are water-soluble at temperatures between 0-35° C.; form a semi-solid gel at physiological temperatures; possess breakable bonds that permit a programmable change in gel properties as a function of time following exposure to an external trigger; and are solutions within a 0-35° C. temperature range and which form a continuous gel at physiological temperatures.

Accordingly, a continuing need exists for the development of new thermo-responsive gels that can be programmed to react to biological activity and which provide precise drug and cellular delivery vehicles and other related uses.

### SUMMARY

Provided herein are polymer compositions comprising non-cross linked polymer blocks, wherein the non-cross linked polymer blocks comprise a labile bond separating one or more polymer subunits. In embodiments, the polymer compositions form a semi-solid gel at physiological temperatures. Provided herein are pharmaceutical compositions comprising a therapeutic agent and the polymer compositions described herein. Provided herein are methods for delivering a therapeutic agent to a subject in need thereof, where the methods include administering the polymer compositions and pharmaceutical compositions described herein to the subject. Provided herein are kits, syringes, catheters, and bio-inks comprising the polymer compositions and pharmaceutical compositions described herein.

### DETAILED DESCRIPTION

Provided herein, inter alia, are non-crosslinked di-block, triblock or multi-block copolymers that possess thermoresponsive properties where the polymers are water-soluble and form a gel at physiological temperatures. These polymers possess a labile bond or bonds that break upon application of an external trigger or by hydrolysis.

In aspects, the polymers described herein can have medical uses as part of devices (for example, syringes or catheters) with no active agent, as drug carriers, or as vehicles for cell support and delivery. For drug or cell delivery, the rate of de-gelation can be programmed so that the gel, after formation at physiological temperature, may gradually or instantly lose its gel properties to allow faster release of entrapped cells or drugs from the polymer. As such, the polymers can be tailored according to the needs of a specific application, in order to either immediately or gradually release therapeutic payloads. Of specific interest is the delivery of therapeutic stem cells carrying anticancer cargo for delivery to cancerous tissue, such as tumors. Therapeutic cells embedded or included in the polymer compositions disclosed herein can be delivered to the site of treatment by administering a water-soluble solution containing the cells that instantly gels upon contacting the target tissue due to reaching physiological temperatures followed by the programmed breakdown of gel properties, allowing the cells to move towards the target tissue.

Definitions

As used herein, “polymers” are molecules containing multiple (typically on the order of 5, 10, 100, 1000 or more) copies of one or more constitutional units, commonly referred to as monomers. As used herein, “homopolymers” are polymers that contain multiple copies of a single constitutional unit. “Copolymers” are polymers that contain multiple copies of at least two dissimilar constitutional units.

As used herein, the terms “polymer composition” and “copolymer composition” are used interchangeably and are meant to include at least one synthesized polymer or copolymer with or without residues from initiators, solvents or other elements attendant to the synthesis of such polymers, where such residues are understood as not being covalently incorporated thereto. A polymer composition can also include materials added after synthesis of the polymer to provide or modify specific properties of such composition.

A “polymer chain” or “polymer strand” is a linear (unbranched) grouping of constitutional repeating units (i.e., a linear block).

A “non-cross linked polymer,” as used herein, refers to a polymer that does not dissolve in water but, rather, swells in water due to excess covalent bonds between individual polymer strands (e.g. polymer chains) that form an insoluble network.

As used herein, a polymer “block” is a portion of a polymer which corresponds to a grouping of constitutional units, for example, 10, 25, 50, 100, 250, 500, 1000, or even more units. Blocks can be branched or unbranched. Blocks can contain a single type of constitutional unit (also referred to herein as “homopolymeric blocks”) or multiple types of constitutional units (also referred to herein as “copolymeric blocks”) which may be provided, for example, in a random, statistical, gradient, or periodic (e.g., alternating) distribution.

A “di-block copolymer” refers to a copolymer having two different homopolymer subunits. An exemplary di-block copolymer is a copolymer of polylactic acid (PLA) and polyethylene glycol (PEG).

A “tri-block copolymer” refers to a copolymer having three distinct subunits. These distinct subunits may be three different homopolymers or the distinct subunits may be two different homopolymers where the end homopolymer subunits are the same and the middle homopolymer subunit is different. An exemplary tri-block copolymer comprises polylactic acid (PLA) and polyethylene glycol (PEG), where such tri-block copolymer may be arranged as PLA-PEG-PLA or PEG-PLA-PEG.

A “multi-block copolymer” refers to a copolymer having multiple distinct subunits. The subunits may be alternating or periodic. An exemplary multi-block copolymer comprises polylactic acid (PLA) and polyethylene glycol (PEG), where such multi-block copolymer may be PLA-PEG-PLA-PEG.

“Number average molecular weight” or “Mn” refer to the statistical average molecular weight of all the polymer chains in a sample, and is defined by Mn=(ΣNᵢMᵢ)/(ΣNᵢ), where Mᵢ is the molecular weight of a chain and Nᵢ is the number of chains of that molecular weight. Number average molecular weight can be predicted by polymerization mechanisms or can be measured by methods known in the art, such as gel-permeation chromatography (optionally coupled with a light scattering detector) or nuclear magnetic resonance or by reference to a polystyrene standard.

A “semi-solid” as used herein can be a gel, a colloid, or a gum. As used herein, semi-solids and liquids are fluids distinguished on the basis of viscosity: a semi-solid is a high viscosity fluid, while a liquid has lower viscosity. A semi-solid (for example, a semi-solid gel) can, in certain embodiments, have a viscosity as high as thousands of mPa·s.

“Water-soluble” refers to the ability of a material to be dissolved, dispersed, swollen, hydrated, or similarly admixed in water. Similarly, as used herein, reference to the phrase “dissolved,” “dissolving” and the like refers to the dissolution, dispersion, swelling, hydration and the like admixture of a material in a liquid medium (e.g., water). In embodiments, “water-soluble” refers to a material dissolved in water (i.e., in solution).

A “labile bond” or a “cleavable bond” is a covalent bond, other than a covalent bond to a hydrogen atom, that is capable of being selectively broken or cleaved under conditions that will not break or cleave other covalent bonds (e.g. non-labile bonds) in the same molecule. More specifically, a labile bond is a covalent bond that is less stable (thermodynamically) or more rapidly broken (kinetically) under appropriate conditions than other non-labile covalent bonds in the same molecule. Cleavage of a labile bond within a molecule may result in the formation of two or more molecules. For those skilled in the art, cleavage or lability of a bond is generally discussed in terms of half-life (tv2) of bond cleavage (the time required for half of the bonds to cleave). A labile bond can be sensitive to pH, oxidative or reductive conditions or agents, temperature, salt concentration, light, sound, the presence of an enzyme (such as esterases, including nucleases, and proteases), or the presence of an added agent. For example, increased or decreased pH is the appropriate condition for a pH-labile bond.

“Physiologically labile bond” is a labile bond that is cleavable under conditions normally encountered or analogous to those encountered within a mammalian body. Physiologically labile linkage groups are selected such that they undergo a chemical transformation (e.g., cleavage) when present in certain physiological conditions (such as a tumor microenvironment).

“Physiological temperature” refers to temperatures between about 35° C. and about 42° C., for example, about 35° C., about 36° C., about 37° C., about 38° C., about 39° C., about 40° C., about 41° C., or about 42° C. In some embodiments, physiological temperature refers to the average physiological temperature of a human (e.g., about 37° C.). In other embodiments, physiological temperature refers to the average physiological temperature of a mammal, for example, a companion animal such as a dog or a cat or an agricultural animal such as cows, sheep, horses, or pigs. In embodiments, physiological temperature is about 37° C. In embodiments, physiological temperature is from about 36.5° C. to about 37.5° C. In embodiments, physiological temperature is from about 36° C. to about 38° C. In embodiments, physiological temperature is from about 35° C. to about 39° C. In embodiments, physiological temperature is about 38° C. In embodiments, physiological temperature is about 39° C.

An “individual” or “subject” can be a vertebrate, a mammal, or a human. Mammals include, but are not limited to, farm animals (such as cows), sport animals, pets (such as horses), primates, mice and rats. Individuals also include companion animals including, but not limited to, dogs and cats. In one aspect, an individual is a human.

An “effective amount” refers to an amount of therapeutic compound, such as an anticancer therapy, administered to an individual, either as a single dose or as part of a series of doses, which is effective to produce a desired therapeutic or prophylactic result.

A “therapeutically effective amount” is at least the minimum concentration required to effect a measurable improvement of a particular disorder. A therapeutically effective amount herein may vary according to factors such as the disease state, age, sex, and weight of the patient, and the ability of the therapeutic to elicit a desired response in the individual. A therapeutically effective amount may also be one in which any toxic or detrimental effects of the therapeutic are outweighed by the therapeutically beneficial effects. In the case of cancer, the therapeutically effective amount may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis: inhibit, to some extent, tumor growth; and/or relieve to some extent one or more of the symptoms associated with the cancer. To the extent the therapeutic may prevent growth and/or kill existing cancer cells, it may be cytostatic and/or cytotoxic.

Administration “in combination with” one or more further therapeutic agents includes simultaneous (concurrent) and consecutive administration in any order.

The term “pharmaceutical composition” or “pharmaceutical formulation” refers to a preparation that is in such form as to permit the biological activity of the active ingredient to be effective, and that generally contains one or more pharmaceutically acceptable excipients. Such formulations are generally sterile.

“Pharmaceutically acceptable excipient” refers to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A “sterile” formulation is aseptic or free from all living microorganisms and their spores.

The term “package insert” is used to refer to instructions customarily included in commercial packages of therapeutic products, that contain information about the indications, usage, dosage, administration, contraindications and/or warnings concerning the use of such therapeutic products.

As used herein, the term “protein” includes polypeptides, peptides, fragments of polypeptides, and fusion polypeptides.

As used herein, a “nucleic acid” refers to two or more deoxyribonucleotides and/or ribonucleotides covalently joined together in either single or double-stranded form.

The term “about” as used herein refers to the usual error range for the respective value readily known to the skilled person in this technical field.

As used herein, the singular terms “a,” “an,” and “the” include the plural reference unless the context clearly indicates otherwise.

Unless defined otherwise herein, all technical and scientific terms used herein have the same meaning as commonly understood by one of ordinary skill in the art to which this invention pertains.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Thermoresponsive Gels

Provided herein are non-crosslinked polymers that possess thermoresponsive properties, where the polymers are water-soluble at temperatures less than a physiological temperature and/or ex vivo, and that form a gel in vivo and/or at physiological temperatures. In embodiments, “less than a physiological temperature” is a temperature less than 37° C. In embodiments, “less than a physiological temperature” is a temperature equal to or less than 36° C. In embodiments, “less than a physiological temperature” is a temperature equal to or less than 35° C. In embodiments, “less than a physiological temperature” is a temperature from about 0° C. to 35° C. In embodiments, “less than a physiological temperature” refers to room temperature. In embodiments, room temperature is from about 15° C. to about 30° C. In embodiments, room temperature is from about 20° C. to about 25° C.

These polymers and copolymers possess a labile or cleavable bond or bonds that break upon application of an external trigger or by hydrolysis. The cleavage of these bonds results in the collapse (i.e., the degradation) of the gel to either a precipitate of the resultant polymer blocks that are not gel forming or the polymer blocks being dissolved in water. The polymers and copolymers may be represented by the formula such as P1-R-P2 or P1-R-P2-R-P1 wherein R is a labile bond and P1 and P2 are different polymer subunits. The skilled artisan will envision the numerous iterations that could be planned with different polymers and labile bonds.

In aspects, the polymers are block copolymers comprising: (i) one or more hydrophilic polymer blocks, (ii) one or more relatively hydrophobic polymer blocks, and (iii) one or more labile bonds. “Relatively hydrophobic” means that the polymer block is more hydrophobic or possesses more hydrophobic properties relative to the hydrophilic polymer block. As used herein, “hydrophilic”, “hydrophilicity” or similar terminology is used to describe substrates (e.g., polymers, copolymers, or polymer blocks) that can be wet by water, and other water-based solutions, such as by aqueous solutions of acids and bases and by polar liquids. For example, the hydrophobic polymer block can be about 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, or 90-100%, such as any of about 5%, 10%, 15%, 20%, 25%, 30%, 35%, 40%, 45%, 50%, 55%, 60%, 65%, 70%, 75%, 80%, 85%, 90%, 95%, 100%, 105%, 110%, or more hydrophobic relative to the corresponding hydrophilic polymer blocks in the copolymer. In embodiments, the block copolymers are di-block copolymers. In embodiments, the block copolymers are tri-block copolymers. In embodiments, the block copolymers are multi-block copolymers.

In aspects, the copolymer comprises hydrophilic blocks, relatively hydrophobic blocks, and one or more labile bonds. In embodiments, the hydrophilic blocks comprise ethylene glycol, 1,2-propylene glycol, 1,3-propylene glycol, 1,4-butanediol, or a combination of two or more thereof. In embodiments, the hydrophilic blocks comprise ethylene glycol. In embodiments, the hydrophilic blocks comprise 1,2-propylene glycol. In embodiments, the hydrophilic blocks comprise 1,3-propylene glycol. In embodiments, the hydrophilic blocks comprise 1,4-butanediol. In embodiments, the hydrophilic blocks comprise two of ethylene glycol, 1,2-propylene glycol, 1,3-propylene glycol, and 1,4-butanediol. In embodiments, the hydrophilic blocks comprise three of ethylene glycol, 1,2-propylene glycol, 1,3-propylene glycol, and 1,4-butanediol. In embodiments, the hydrophilic blocks comprise ethylene glycol, 1,2-propylene glycol, 1,3-propylene glycol, and 1,4-butanediol.

In aspects, the relatively hydrophobic blocks comprise lactic acid, glycolic acid, caprolactone, hydroxybutyric acid, hydroxyvaleric acid, hydroxycaproic acid, or a combination of two or more thereof. In embodiments, the relatively hydrophobic blocks comprise lactic acid. In embodiments, the relatively hydrophobic blocks comprise glycolic acid. In embodiments, the relatively hydrophobic blocks comprise hydroxybutyric acid. In embodiments, the relatively hydrophobic blocks comprise hydroxyvaleric acid. In embodiments, the relatively hydrophobic blocks comprise hydroxycaproic acid. In embodiments, the relatively hydrophobic blocks comprise caprolactone. In embodiments, the relatively hydrophobic blocks comprise lactic acid and glycolic acid. In embodiments, the relatively hydrophobic blocks comprise two of lactic acid, glycolic acid, hydroxybutyric acid, hydroxyvaleric acid, hydroxycaproic acid, and caprolactone. In embodiments, the relatively hydrophobic blocks comprise three of lactic acid, glycolic acid, hydroxybutyric acid, hydroxyvaleric acid, hydroxycaproic acid, and caprolactone. In embodiments, the relatively hydrophobic blocks comprise four of lactic acid, glycolic acid, hydroxybutyric acid, hydroxyvaleric acid, hydroxycaproic acid, and caprolactone. In embodiments, the relatively hydrophobic blocks comprise lactic acid, glycolic acid, hydroxybutyric acid, hydroxyvaleric acid, hydroxycaproic acid, and caprolactone.

In aspects, the copolymer comprises: (a) ethylene glycol, 1,2-propylene glycol, 1,3-propylene glycol, 1,4-butanediol, or a combination of two or more thereof; and (b) lactic acid, glycolic acid, caprolactone, hydroxybutyric acid, hydroxyvaleric acid, hydroxycaproic acid, or a combination of two or more thereof. In aspects, the copolymer comprises: (a) ethylene glycol; and (b) 1,2-propylene glycol, 1,3-propylene glycol, 1,4-butanediol, lactic acid, glycolic acid, caprolactone, hydroxybutyric acid, hydroxyvaleric acid, hydroxycaproic acid, or a combination of two or more thereof. In embodiments, the copolymer comprises: (a) ethylene glycol; and (b) 1,2-propylene glycol, 1,3-propylene glycol, 1,4-butanediol, or a combination of two or more thereof. In embodiments, the copolymer comprises: (a) ethylene glycol; and (b) 1,2-propylene glycol, 1,3-propylene glycol, or a combination thereof. In embodiments, the copolymer comprises: (a) ethylene glycol; and (b) 1,2-propylene glycol. In embodiments, the copolymer comprises: (a) ethylene glycol; and (b) 1,3-propylene glycol. In embodiments, the copolymer comprises: (a) ethylene glycol; and (b) lactic acid, glycolic acid, caprolactone, hydroxybutyric acid, hydroxyvaleric acid, hydroxycaproic acid, or a combination of two or more thereof. In embodiments, the copolymer comprises: (a) ethylene glycol; and (b) lactic acid, glycolic acid, caprolactone, or a combination of two or more thereof. In embodiments, the copolymer comprises: (a) ethylene glycol; and (b) caprolactone. In embodiments, the copolymer comprises: (a) ethylene glycol; and (b) lactic acid. In embodiments, the copolymer comprises: (a) ethylene glycol; and (b) glycolic acid. In embodiments, the copolymer comprises: (a) ethylene glycol; and (b) a combination of lactic acid and glycolic acid (e.g., poly(lactic-co-glycolic acid) or poly(lactide-co-glycolide)). In each instance, the copolymers described herein comprise one or more labile bonds. In embodiments, the ethylene glycol is polyethylene glycol. In embodiments, the 1,2-propylene glycol is poly(1,2-propylene glycol). In embodiments, the 1,3-propylene glycol is poly(1,3-propylene glycol). In embodiments, the 1,4-butanediol is poly(1,4-butanediol). In embodiments, the lactic acid is polylactic acid. In embodiments, the glycolic acid is polyglycolic acid. In embodiments, the caprolactone is polycaprolactone. In embodiments, the hydroxybutyric acid is polyhydroxybutyric acid. In embodiments, the hydroxyvaleric acid is polyhydroxyvaleric acid. In embodiments, the hydroxycaproic acid is polyhydrocaproic acid. In embodiments, the polyethylene glycol has a number average molecular weight from about 100 Daltons to about 10,000 Daltons. In embodiments, the polyethylene glycol has a number average molecular weight from about 100 Daltons to about 8,000 Daltons. In embodiments, the polyethylene glycol has a number average molecular weight from about 500 Daltons to about 5,000 Daltons. In embodiments, the polyethylene glycol has a number average molecular weight from about 1,000 Daltons to about 4,000 Daltons. In embodiments, the polyethylene glycol has a number average molecular weight from about 1,500 Daltons to about 10,000 Daltons. In embodiments, the polyethylene glycol has a number average molecular weight from about 2,000 Daltons to about 4,000 Daltons. In embodiments, the polyethylene glycol has a number average molecular weight from about 1,500 Daltons to about 2,500 Daltons. In embodiments, the polyethylene glycol has a number average molecular weight from about 3,000 Daltons to about 4,000 Daltons. In embodiments, the polyethylene glycol has a number average molecular weight of about 2,000 Daltons. In embodiments, the polyethylene glycol has a number average molecular weight of about 3,500 Daltons. In embodiments, the polylactic acid has a number average molecular weight from about 100 Daltons to about 10,000 Daltons. In embodiments, the polylactic acid has a number average molecular weight from about 500 Daltons to 5,000 Dalton. In embodiments, the polylactic acid has a number average molecular weight from about 1,000 Daltons to about 4,000 Daltons. In embodiments, the polylactic acid has a number average molecular weight from about 2,000 Daltons to about 4,000 Daltons. In embodiments, the polylactic acid has a number average molecular weight from about 2,000 Daltons to about 3,000 Daltons. In embodiments, the polylactic acid has a number average molecular weight from about 3,000 Daltons to about 4,000 Daltons. In embodiments, the polyglycolic acid has a number average molecular weight from about 100 Daltons to about 10,000 Daltons. In embodiments, the polyglycolic acid has a number average molecular weight from about 500 Daltons to 5,000 Dalton. In embodiments, the polyglycolic acid has a number average molecular weight from about 1,000 Daltons to about 4,000 Daltons. In embodiments, the polyglycolic acid has a number average molecular weight from about 2,000 Daltons to about 4,000 Daltons. In embodiments, the polyglycolic acid has a number average molecular weight from about 2,000 Daltons to about 3,000 Daltons. In embodiments, the polyglycolic acid has a number average molecular weight from about 3,000 Daltons to about 4,000 Daltons. In embodiments, the weight ratio of lactic acid to glycolic acid in the poly(lactic-co-glycolic acid) is from about 95:5 to about 5:95. In embodiments, the weight ratio of lactic acid to glycolic acid in the poly(lactic-co-glycolic acid) is from about 95:5 to about 50:50. In embodiments, the weight ratio of lactic acid to glycolic acid in the poly(lactic-co-glycolic acid) is from about 90:10 to about 50:50. In embodiments, the weight ratio of lactic acid to glycolic acid in the poly(lactic-co-glycolic acid) is from about 90:10 to about 60:40. In embodiments, the weight ratio of lactic acid to glycolic acid in the poly(lactic-co-glycolic acid) is from about 80:20 to about 50:50. In embodiments, the weight ratio of lactic acid to glycolic acid in the poly(lactic-co-glycolic acid) is from about 80:20 to about 60:40. in embodiments, the weight ratio of lactic acid to glycolic acid in the poly(lactic-co-glycolic acid) is about 50:50. In embodiments, the weight ratio of lactic acid to glycolic acid in the poly(lactic-co-glycolic acid) is about 60:40. In embodiments, the weight ratio of lactic acid to glycolic acid in the poly(lactic-co-glycolic acid) is about 65:35. In embodiments, the weight ratio of lactic acid to glycolic acid in the poly(lactic-co-glycolic acid) is about 70:30. In embodiments, the weight ratio of lactic acid to glycolic acid in the poly(lactic-co-glycolic acid) is about 75:25. In embodiments, the weight ratio of lactic acid to glycolic acid in the poly(lactic-co-glycolic acid) is about 80:20. In embodiments, the weight ratio of lactic acid to glycolic acid in the poly(lactic-co-glycolic acid) is about 85:15. In embodiments, the weight ratio of lactic acid to glycolic acid in the poly(lactic-co-glycolic acid) is about 90:10. In embodiments, the poly(lactic-co-glycolic acid) has a number average molecular weight from about 100 Daltons to about 10,000 Daltons. In embodiments, the poly(lactic-co-glycolic acid) has a number average molecular weight from about 500 Daltons to 5,000 Dalton. In embodiments, the poly(lactic-co-glycolic acid) has a number average molecular weight from about 1,000 Daltons to about 4,000 Daltons. In embodiments, the poly(lactic-co-glycolic acid) has a number average molecular weight from about 2,000 Daltons to about 4,000 Daltons. In embodiments, the poly(lactic-co-glycolic acid) has a number average molecular weight from about 2,000 Daltons to about 3,000 Daltons. In embodiments, the poly(lactic-co-glycolic acid) has a number average molecular weight from about 3,000 Daltons to about 4,000 Daltons. In embodiments, the poly(lactic-co-glycolic acid) has a number average molecular weight from about 3,500 Daltons to about 4,000 Daltons. In embodiments, the poly(1,2-propylene glycol) has a number average molecular weight from about 100 Daltons to about 10,000 Daltons. In embodiments, the poly(1,2-propylene glycol) has a number average molecular weight from about 500 Daltons to 5,000 Dalton. In embodiments, the poly(1,2-propylene glycol) has a number average molecular weight from about 1,000 Daltons to about 4,000 Daltons. In embodiments, the poly(1,2-propylene glycol) has a number average molecular weight from about 1,000 Daltons to about 3,000 Daltons. In embodiments, the poly(1,2-propylene glycol) has a number average molecular weight from about 1,000 Daltons to about 2,000 Daltons. In embodiments, the poly(1,2-propylene glycol) has a number average molecular weight from about 2,000 Daltons to about 4,000 Daltons. In embodiments, the poly(1,2-propylene glycol) has a number average molecular weight from about 2,000 Daltons to about 3,000 Daltons. In embodiments, the poly(1,2-propylene glycol) has a number average molecular weight from about 3,000 Daltons to about 4,000 Daltons. In embodiments, the poly(1,3-propylene glycol) has a number average molecular weight from about 100 Daltons to about 10,000 Daltons. In embodiments, the poly(1,3-propylene glycol) has a number average molecular weight from about 500 Daltons to 5,000 Dalton. In embodiments, the poly(1,3-propylene glycol) has a number average molecular weight from about 1,000 Daltons to about 4,000 Daltons. In embodiments, the poly(1,3-propylene glycol) has a number average molecular weight from about 1,000 Daltons to about 3,000 Daltons. In embodiments, the poly(1,3-propylene glycol) has a number average molecular weight from about 1,000 Daltons to about 2,000 Daltons. In embodiments, the poly(1,3-propylene glycol) has a number average molecular weight from about 2,000 Daltons to about 4,000 Daltons. In embodiments, the poly(1,3-propylene glycol) has a number average molecular weight from about 2,000 Daltons to about 3,000 Daltons. In embodiments, the poly(1,3-propylene glycol) has a number average molecular weight from about 3,000 Daltons to about 4,000 Daltons. In embodiments, the polycaprolactone has a number average molecular weight from about 100 Daltons to about 10,000 Daltons. In embodiments, the polycaprolactone has a number average molecular weight from about 500 Daltons to 5,000 Dalton. In embodiments, the polycaprolactone has a number average molecular weight from about 1,000 Daltons to about 4,000 Daltons. In embodiments, the polycaprolactone has a number average molecular weight from about 1,000 Daltons to about 3,000 Daltons. In embodiments, the polycaprolactone has a number average molecular weight from about 1,000 Daltons to about 2,000 Daltons. In embodiments, the polycaprolactone has a number average molecular weight from about 2,000 Daltons to about 4,000 Daltons. In embodiments, the polycaprolactone has a number average molecular weight from about 2,000 Daltons to about 3,000 Daltons. In embodiments, the polycaprolactone has a number average molecular weight from about 3,000 Daltons to about 4,000 Daltons. In embodiments, the poly(1,4-butanediol) has a number average molecular weight from about 100 Daltons to about 10,000 Daltons. In embodiments, the poly(1,4-butanediol) has a number average molecular weight from about 500 Daltons to 5,000 Dalton. In embodiments, the poly(1,4-butanediol) has a number average molecular weight from about 1,000 Daltons to about 4,000 Daltons. In embodiments, the poly(1,4-butanediol) has a number average molecular weight from about 1,000 Daltons to about 3,000 Daltons. In embodiments, the poly(1,4-butanediol) has a number average molecular weight from about 1,000 Daltons to about 2,000 Daltons. In embodiments, the poly(1,4-butanediol) has a number average molecular weight from about 2,000 Daltons to about 4,000 Daltons. In embodiments, the poly(1,4-butanediol) has a number average molecular weight from about 2,000 Daltons to about 3,000 Daltons. In embodiments, the poly(1,4-butanediol) has a number average molecular weight from about 3,000 Daltons to about 4,000 Daltons. In embodiments, the polyhydroxybutric acid has a number average molecular weight from about 100 Daltons to about 10,000 Daltons. In embodiments, the polyhydroxybutric acid has a number average molecular weight from about 500 Daltons to 5,000 Dalton. In embodiments, the polyhydroxybutric acid has a number average molecular weight from about 1,000 Daltons to about 4,000 Daltons. In embodiments, the polyhydroxybutric acid has a number average molecular weight from about 1,000 Daltons to about 3,000 Daltons. In embodiments, the polyhydroxybutric acid has a number average molecular weight from about 1,000 Daltons to about 2,000 Daltons. In embodiments, the polyhydroxybutric acid has a number average molecular weight from about 2,000 Daltons to about 4,000 Daltons. In embodiments, the polyhydroxybutric acid has a number average molecular weight from about 2,000 Daltons to about 3,000 Daltons. In embodiments, the polyhydroxybutric acid has a number average molecular weight from about 3,000 Daltons to about 4,000 Daltons. In embodiments, the polyhydroxyvaleric acid has a number average molecular weight from about 100 Daltons to about 10,000 Daltons. In embodiments, the polyhydroxyvaleric acid has a number average molecular weight from about 500 Daltons to 5,000 Dalton. In embodiments, the polyhydroxyvaleric acid has a number average molecular weight from about 1,000 Daltons to about 4,000 Daltons. In embodiments, the polyhydroxyvaleric acid has a number average molecular weight from about 1,000 Daltons to about 3,000 Daltons. In embodiments, the polyhydroxyvaleric acid has a number average molecular weight from about 1,000 Daltons to about 2,000 Daltons. In embodiments, the polyhydroxyvaleric acid has a number average molecular weight from about 2,000 Daltons to about 4,000 Daltons. In embodiments, the polyhydroxyvaleric acid has a number average molecular weight from about 2,000 Daltons to about 3,000 Daltons. In embodiments, the polyhydroxyvaleric acid has a number average molecular weight from about 3,000 Daltons to about 4,000 Daltons. In embodiments, the polyhydroxycaproic acid has a number average molecular weight from about 100 Daltons to about 10,000 Daltons. In embodiments, the polyhydroxycaproic acid has a number average molecular weight from about 500 Daltons to 5,000 Dalton. In embodiments, the polyhydroxycaproic acid has a number average molecular weight from about 1,000 Daltons to about 4,000 Daltons. In embodiments, the polyhydroxycaproic acid has a number average molecular weight from about 1,000 Daltons to about 3,000 Daltons. In embodiments, the polyhydroxycaproic acid has a number average molecular weight from about 1,000 Daltons to about 2,000 Daltons. In embodiments, the polyhydroxycaproic acid has a number average molecular weight from about 2,000 Daltons to about 4,000 Daltons. In embodiments, the polyhydroxycaproic acid has a number average molecular weight from about 2,000 Daltons to about 3,000 Daltons. In embodiments, the polyhydroxycaproic acid has a number average molecular weight from about 3,000 Daltons to about 4,000 Daltons.

In aspects, the copolymer is a di-block copolymer comprising polylactic acid (PLA) and polyethylene glycol (PEG). In embodiments, the copolymer is a tri-block copolymer comprising polylactic acid and polyethylene glycol. In embodiments, the tri-block copolymer comprises polylactic acid-polyethylene glycol-polylactic acid. In embodiments, the tri-block copolymer comprises polyethylene glycol-polylactic acid-polyethylene glycol. In embodiments, the copolymer is a multi-block copolymer comprising polylactic acid and polyethylene glycol. As described herein, at least two polymers are linked by a cleavable labile group (described as “R”) such that the disclosure encompasses structures such as, e.g., PLA-R-PEG; PLA-R-PEG-PLA; PLA-R-PEG-R-PLA; PEG-R-PLA-R-PEG; PEG-R-PLA-PEG; PLA-PEG-R-PEG-PLA; PLA-R-PEG-R-PEG-PLA; PLA-R-PEG-R-PEG-R-PLA, and the like. In embodiments, the polylactic acid is a poly(D,L-lactic acid). In embodiments, the polylactic acid is a poly(L-lactic acid). In embodiments, the polyethylene glycol has a number average molecular weight from about 100 Daltons to about 10,000 Daltons. In embodiments, the polyethylene glycol has a number average molecular weight from about 100 Daltons to about 8,000 Daltons. In embodiments, the polyethylene glycol has a number average molecular weight from about 500 Daltons to about 5,000 Daltons. In embodiments, the polyethylene glycol has a number average molecular weight from about 1,000 Daltons to about 4,000 Daltons. In embodiments, the polyethylene glycol has a number average molecular weight from about 1,500 Daltons to about 10,000 Daltons. In embodiments, the polyethylene glycol has a number average molecular weight from about 2,000 Daltons to about 4,000 Daltons. In embodiments, the polyethylene glycol has a number average molecular weight from about 1,500 Daltons to about 2,500 Daltons. In embodiments, the polyethylene glycol has a number average molecular weight from about 3,000 Daltons to about 4,000 Daltons. In embodiments, the polyethylene glycol has a number average molecular weight of about 2,000 Daltons. In embodiments, the polyethylene glycol has a number average molecular weight of about 3,500 Daltons. In embodiments, the polylactic acid has a number average molecular weight from about 100 Daltons to about 10,000 Daltons. In embodiments, the polylactic acid has a number average molecular weight from about 500 Daltons to 5,000 Dalton. In embodiments, the polylactic acid has a number average molecular weight from about 1,000 Daltons to about 4,000 Daltons. In embodiments, the polylactic acid has a number average molecular weight from about 2,000 Daltons to about 4,000 Daltons. In embodiments, the polylactic acid has a number average molecular weight from about 2,000 Daltons to about 3,000 Daltons. In embodiments, the polylactic acid has a number average molecular weight from about 3,000 Daltons to about 4,000 Daltons. The cleavable linking group R is as described further herein.

In aspects, the copolymer is a di-block copolymer comprising polyglycolic acid (PGA) and polyethylene glycol (PEG). In embodiments, the copolymer is a tri-block copolymer comprising polyglycolic acid and polyethylene glycol. In embodiments, the tri-block copolymer comprises polyglycolic acid-polyethylene glycol-polyglycolic acid. In embodiments, the tri-block copolymer comprises polyethylene glycol-polyglycolic acid-polyethylene glycol. In embodiments, the copolymer is a multi-block copolymer comprising polyglycolic acid and polyethylene glycol. As described herein, at least two copolymers are linked by a cleavable labile group (described as “R”) such that the disclosure encompasses structures such as, e.g., PGA-R-PEG; PGA-R-PEG-PGA; PGA-R-PEG-R-PGA; PEG-R-PGA-R-PEG; PEG-R-PGA-PEG; PGA-PEG-R-PEG-PGA; PGA-R-PEG-R-PEG-PGA; PGA-R-PEG-R-PEG-R-PGA, and the like. In embodiments, the polyglycolic acid is a poly(D,L-glycolic acid). In embodiments, the polyglycolic acid is a poly(L-glycolic acid). In embodiments, the polyethylene glycol has a number average molecular weight from about 100 Daltons to about 10,000 Daltons. In embodiments, the polyethylene glycol has a number average molecular weight from about 100 Daltons to about 8,000 Daltons. In embodiments, the polyethylene glycol has a number average molecular weight from about 500 Daltons to about 5,000 Daltons. In embodiments, the polyethylene glycol has a number average molecular weight from about 1,000 Daltons to about 4,000 Daltons. In embodiments, the polyethylene glycol has a number average molecular weight from about 1,500 Daltons to about 10,000 Daltons. In embodiments, the polyethylene glycol has a number average molecular weight from about 2,000 Daltons to about 4,000 Daltons. In embodiments, the polyethylene glycol has a number average molecular weight from about 1,500 Daltons to about 2,500 Daltons. In embodiments, the polyethylene glycol has a number average molecular weight from about 3,000 Daltons to about 4,000 Daltons. In embodiments, the polyethylene glycol has a number average molecular weight of about 2,000 Daltons. In embodiments, the polyethylene glycol has a number average molecular weight of about 3,500 Daltons. In embodiments, the polyglycolic acid has a number average molecular weight from about 100 Daltons to about 10,000 Daltons. In embodiments, the polyglycolic acid has a number average molecular weight from about 500 Daltons to 5,000 Dalton. In embodiments, the polyglycolic acid has a number average molecular weight from about 1,000 Daltons to about 4,000 Daltons. In embodiments, the polyglycolic acid has a number average molecular weight from about 2,000 Daltons to about 4,000 Daltons. In embodiments, the polyglycolic acid has a number average molecular weight from about 2,000 Daltons to about 3,000 Daltons. In embodiments, the polyglycolic acid has a number average molecular weight from about 3,000 Daltons to about 4,000 Daltons. The cleavable linking group R is as described further herein.

In aspects, the copolymer is a di-block copolymer comprising a poly(lactic-co-glycolic acid) (PLGA) and polyethylene glycol (PEG). In embodiments, the copolymer is tri-block copolymer comprising a poly(lactic-co-glycolic acid) and polyethylene glycol. In embodiments, the tri-block copolymer comprises poly(lactic-co-glycolic acid)-polyethylene glycol-poly(lactic-co-glycolic acid). In embodiments, the tri-block copolymer comprises polyethylene glycol-poly(lactic-co-glycolic acid)-polyethylene glycol. In embodiments, the copolymer is a multi-block copolymer comprising a poly(lactic-co-glycolic acid) and polyethylene glycol. As described herein, at least two copolymers are linked by a cleavable labile group (described as “R”) such that the disclosure encompasses structures such as, e.g., PLGA-R-PEG; PLGA-R-PEG-PLGA; PLGA-R-PEG-R-PLGA; PEG-R-PLGA-R-PEG; PEG-R-PLGA-PEG; PLGA-PEG-R-PEG-PLGA; PLGA-R-PEG-R-PEG-PLGA; PLGA-R-PEG-R-PEG-R-PLGA, and the like. In embodiments, the poly(lactic-co-glycolic acid) is a poly(D,L-lactic-co-glycolic acid). In embodiments, the weight ratio of lactic acid to glycolic acid in the poly(lactic-co-glycolic acid) is from about 95:5 to about 5:95. In embodiments, the weight ratio of lactic acid to glycolic acid in the poly(lactic-co-glycolic acid) is from about 95:5 to about 50:50. In embodiments, the weight ratio of lactic acid to glycolic acid in the poly(lactic-co-glycolic acid) is from about 90:10 to about 50:50. in embodiments, the weight ratio of lactic acid to glycolic acid in the poly(lactic-co-glycolic acid) is from about 90:10 to about 60:40. In embodiments, the weight ratio of lactic acid to glycolic acid in the poly(lactic-co-glycolic acid) is from about 80:20 to about 50:50. In embodiments, the weight ratio of lactic acid to glycolic acid in the poly(lactic-co-glycolic acid) is from about 80:20 to about 60:40. In embodiments, the weight ratio of lactic acid to glycolic acid in the poly(lactic-co-glycolic acid) is about 50:50. in embodiments, the weight ratio of lactic acid to glycolic acid in the poly(lactic-co-glycolic acid) is about 60:40. In embodiments, the weight ratio of lactic acid to glycolic acid in the poly(lactic-co-glycolic acid) is about 65:35. in embodiments, the weight ratio of lactic acid to glycolic acid in the poly(lactic-co-glycolic acid) is about 70:30. In embodiments, the weight ratio of lactic acid to glycolic acid in the poly(lactic-co-glycolic acid) is about 75:25. In embodiments, the weight ratio of lactic acid to glycolic acid in the poly(lactic-co-glycolic acid) is about 80:20. In embodiments, the weight ratio of lactic acid to glycolic acid in the poly(lactic-co-glycolic acid) is about 85:15. In embodiments, the weight ratio of lactic acid to glycolic acid in the poly(lactic-co-glycolic acid) is about 90:10. In embodiments, the poly(lactic-co-glycolic acid) has a number average molecular weight from about 100 Daltons to about 10,000 Daltons. In embodiments, the poly(lactic-co-glycolic acid) has a number average molecular weight from about 500 Daltons to 5,000 Dalton. In embodiments, the poly(lactic-co-glycolic acid) has a number average molecular weight from about 1,000 Daltons to about 4,000 Daltons. In embodiments, the poly(lactic-co-glycolic acid) has a number average molecular weight from about 2,000 Daltons to about 4,000 Daltons. In embodiments, the poly(lactic-co-glycolic acid) has a number average molecular weight from about 2,000 Daltons to about 3,000 Daltons. In embodiments, the poly(lactic-co-glycolic acid) has a number average molecular weight from about 3,000 Daltons to about 4,000 Daltons. In embodiments, the poly(lactic-co-glycolic acid) has a number average molecular weight from about 3,500 Daltons to about 4,000 Daltons. In embodiments, the polyethylene glycol has a number average molecular weight from about 100 Daltons to about 10,000 Daltons. In embodiments, the polyethylene glycol has a number average molecular weight from about 100 Daltons to about 8,000 Daltons. In embodiments, the polyethylene glycol has a number average molecular weight from about 500 Daltons to about 5,000 Daltons. In embodiments, the polyethylene glycol has a number average molecular weight from about 1,000 Daltons to about 4,000 Daltons. In embodiments, the polyethylene glycol has a number average molecular weight from about 1,500 Daltons to about 10,000 Daltons. In embodiments, the polyethylene glycol has a number average molecular weight from about 2,000 Daltons to about 4,000 Daltons. In embodiments, the polyethylene glycol has a number average molecular weight from about 1,500 Daltons to about 2,500 Daltons. In embodiments, the polyethylene glycol has a number average molecular weight from about 3,000 Daltons to about 4,000 Daltons. In embodiments, the polyethylene glycol has a number average molecular weight of about 2,000 Daltons. In embodiments, the polyethylene glycol has a number average molecular weight of about 3,500 Daltons. The cleavable linking group R is as described further herein.

In aspects, the copolymer is a di-block copolymer comprising poly(1,2-propylene glycol) (PPG) and polyethylene glycol (PEG). In embodiments, the copolymer is a tri-block copolymer comprising poly(1,2-propylene glycol) and polyethylene glycol. In embodiments, the tri-block copolymer comprises poly(1,2-propylene glycol)-polyethylene glycol-poly(1,2-propylene glycol). In embodiments, the tri-block copolymer comprises polyethylene glycol-poly(1,2-propylene glycol)-polyethylene glycol. In embodiments, the copolymer is a multi-block copolymer comprising poly(1,2-propylene glycol) and polyethylene glycol. As described herein, at least two copolymers are linked by a cleavable labile group (described as “R”) such that the disclosure encompasses structures such as, e.g., PPG-R-PEG; PPG-R-PEG-PPG; PPG-R-PEG-R-PPG; PEG-R-PPG-R-PEG; PEG-R-PPG-PEG; PPG-PEG-R-PEG-PPG; PPG-R-PEG-R-PEG-PPG; PPG-R-PEG-R-PEG-R-PPG, and the like. In embodiments, the polyethylene glycol has a number average molecular weight from about 100 Daltons to about 10,000 Daltons. In embodiments, the polyethylene glycol has a number average molecular weight from about 100 Daltons to about 8,000 Daltons. In embodiments, the polyethylene glycol has a number average molecular weight from about 500 Daltons to about 5,000 Daltons. In embodiments, the polyethylene glycol has a number average molecular weight from about 1,000 Daltons to about 4,000 Daltons. In embodiments, the polyethylene glycol has a number average molecular weight from about 1,500 Daltons to about 10,000 Daltons. In embodiments, the polyethylene glycol has a number average molecular weight from about 2,000 Daltons to about 4,000 Daltons. In embodiments, the polyethylene glycol has a number average molecular weight from about 1,500 Daltons to about 2,500 Daltons. In embodiments, the polyethylene glycol has a number average molecular weight from about 3,000 Daltons to about 4,000 Daltons. In embodiments, the polyethylene glycol has a number average molecular weight of about 2,000 Daltons. In embodiments, the polyethylene glycol has a number average molecular weight of about 3,500 Daltons. In embodiments, the poly(1,2-propylene glycol) has a number average molecular weight from about 100 Daltons to about 10,000 Daltons. In embodiments, the poly(1,2-propylene glycol) has a number average molecular weight from about 500 Daltons to 5,000 Dalton. In embodiments, the poly(1,2-propylene glycol) has a number average molecular weight from about 1,000 Daltons to about 4,000 Daltons. In embodiments, the poly(1,2-propylene glycol) has a number average molecular weight from about 1,000 Daltons to about 3,000 Daltons. In embodiments, the poly(1,2-propylene glycol) has a number average molecular weight from about 1,000 Daltons to about 2,000 Daltons. In embodiments, the poly(1,2-propylene glycol) has a number average molecular weight from about 2,000 Daltons to about 4,000 Daltons. In embodiments, the poly(1,2-propylene glycol) has a number average molecular weight from about 2,000 Daltons to about 3,000 Daltons. In embodiments, the poly(1,2-propylene glycol) has a number average molecular weight from about 3,000 Daltons to about 4,000 Daltons. The cleavable linking group R is as described further herein.

In aspects, the copolymer is a di-block copolymer comprising poly(1,3-propylene glycol) (PPG) and polyethylene glycol (PEG). In embodiments, the copolymer is a tri-block copolymer comprising poly(1,3-propylene glycol) and polyethylene glycol. In embodiments, the tri-block copolymer comprises poly(1,3-propylene glycol)-polyethylene glycol-poly(1,3-propylene glycol). In embodiments, the tri-block copolymer comprises polyethylene glycol-poly(1,3-propylene glycol)-polyethylene glycol. In embodiments, the copolymer is a multi-block copolymer comprising poly(1,3-propylene glycol) and polyethylene glycol. As described herein, at least two copolymers are linked by a cleavable labile group (described as “R”) such that the disclosure encompasses structures such as, e.g., PPG-R-PEG; PPG-R-PEG-PPG; PPG-R-PEG-R-PPG; PEG-R-PPG-R-PEG; PEG-R-PPG-PEG; PPG-PEG-R-PEG-PPG; PPG-R-PEG-R-PEG-PPG; PPG-R-PEG-R-PEG-R-PPG, and the like. In embodiments, the polyethylene glycol has a number average molecular weight from about 100 Daltons to about 10,000 Daltons. In embodiments, the polyethylene glycol has a number average molecular weight from about 100 Daltons to about 8,000 Daltons. In embodiments, the polyethylene glycol has a number average molecular weight from about 500 Daltons to about 5,000 Daltons. In embodiments, the polyethylene glycol has a number average molecular weight from about 1,000 Daltons to about 4,000 Daltons. In embodiments, the polyethylene glycol has a number average molecular weight from about 1,500 Daltons to about 10,000 Daltons. In embodiments, the polyethylene glycol has a number average molecular weight from about 2,000 Daltons to about 4,000 Daltons. In embodiments, the polyethylene glycol has a number average molecular weight from about 1,500 Daltons to about 2,500 Daltons. In embodiments, the polyethylene glycol has a number average molecular weight from about 3,000 Daltons to about 4,000 Daltons. In embodiments, the polyethylene glycol has a number average molecular weight of about 2,000 Daltons. In embodiments, the polyethylene glycol has a number average molecular weight of about 3,500 Daltons. In embodiments, the poly(1,3-propylene glycol) has a number average molecular weight from about 100 Daltons to about 10,000 Daltons. In embodiments, the poly(1,3-propylene glycol) has a number average molecular weight from about 500 Daltons to 5,000 Dalton. In embodiments, the poly(1,3-propylene glycol) has a number average molecular weight from about 1,000 Daltons to about 4,000 Daltons. In embodiments, the poly(1,3-propylene glycol) has a number average molecular weight from about 1,000 Daltons to about 3,000 Daltons. In embodiments, the poly(1,3-propylene glycol) has a number average molecular weight from about 1,000 Daltons to about 2,000 Daltons. In embodiments, the poly(1,3-propylene glycol) has a number average molecular weight from about 2,000 Daltons to about 4,000 Daltons. In embodiments, the poly(1,3-propylene glycol) has a number average molecular weight from about 2,000 Daltons to about 3,000 Daltons. In embodiments, the poly(1,3-propylene glycol) has a number average molecular weight from about 3,000 Daltons to about 4,000 Daltons. The cleavable linking group R is as described further herein.

In aspects, the copolymer is a di-block copolymer comprising polycaprolactone (PCL) and polyethylene glycol (PEG). In embodiments, the copolymer is a tri-block copolymer comprising polycaprolactone and polyethylene glycol. In embodiments, the tri-block copolymer comprises polycaprolactone-polyethylene glycol-polycaprolactone. In embodiments, the tri-block copolymer comprises polyethylene glycol-polycaprolactone-polyethylene glycol. In embodiments, the copolymer is a multi-block copolymer comprising polycaprolactone and polyethylene glycol. As described herein, at least two copolymers are linked by a cleavable labile group (described as “R”) such that the disclosure encompasses structures such as, e.g., PCL-R-PEG; PCL-R-PEG-PCL; PCL-R-PEG-R-PCL; PEG-R-PCL-R-PEG; PEG-R-PCL-PEG; PCL-PEG-R-PEG-PCL; PCL-R-PEG-R-PEG-PCL; PCL-R-PEG-R-PEG-R-PCL, and the like. In embodiments, the polyethylene glycol has a number average molecular weight from about 100 Daltons to about 10,000 Daltons. In embodiments, the polyethylene glycol has a number average molecular weight from about 100 Daltons to about 8,000 Daltons. In embodiments, the polyethylene glycol has a number average molecular weight from about 500 Daltons to about 5,000 Daltons. In embodiments, the polyethylene glycol has a number average molecular weight from about 1,000 Daltons to about 4,000 Daltons. In embodiments, the polyethylene glycol has a number average molecular weight from about 1,500 Daltons to about 10,000 Daltons. In embodiments, the polyethylene glycol has a number average molecular weight from about 2,000 Daltons to about 4,000 Daltons. In embodiments, the polyethylene glycol has a number average molecular weight from about 1,500 Daltons to about 2,500 Daltons. In embodiments, the polyethylene glycol has a number average molecular weight from about 3,000 Daltons to about 4,000 Daltons. In embodiments, the polyethylene glycol has a number average molecular weight of about 2,000 Daltons. In embodiments, the polyethylene glycol has a number average molecular weight of about 3,500 Daltons. In embodiments, the polycaprolactone has a number average molecular weight from about 100 Daltons to about 10,000 Daltons. In embodiments, the polycaprolactone has a number average molecular weight from about 500 Daltons to 5,000 Dalton. In embodiments, the polycaprolactone has a number average molecular weight from about 1,000 Daltons to about 4,000 Daltons. In embodiments, the polycaprolactone has a number average molecular weight from about 1,000 Daltons to about 3,000 Daltons. In embodiments, the polycaprolactone has a number average molecular weight from about 1,000 Daltons to about 2,000 Daltons. In embodiments, the polycaprolactone has a number average molecular weight from about 2,000 Daltons to about 4,000 Daltons. In embodiments, the polycaprolactone has a number average molecular weight from about 2,000 Daltons to about 3,000 Daltons. In embodiments, the polycaprolactone has a number average molecular weight from about 3,000 Daltons to about 4,000 Daltons. The cleavable linking group R is as described further herein.

In aspects, the copolymer is any one of a polylactic acid (PLA)-polyethylene glycol (PEG) di-block copolymer, a PLA-PEG-PLA tri-block copolymer, a PEG-PLA-PEG triblock copolymer, a PEG-PLA multi-block copolymer, a polylactic acid-co-glycolic acid (PLGA)-PEG di-block copolymer, a PLGA-PEG-PLGA tri-block copolymer, a PEG-PLGA-PEG triblock copolymer, a PEG-PLGA multi-block copolymer, a poly(1,2-propylene glycol) (PPG)-PEG di-block copolymer, a PPG-PEG-PPG tri-block copolymer, a PEG-PPG-PEG triblock copolymer, a PEG-PPG multi-block copolymer, polycaprolactone (PCL)-PEG di-block copolymer, a PCL-PEG-PCL tri-block copolymer, a PEG-PCL-PEG triblock copolymer, a PEG-PCL multi-block copolymer, or a combination of two or more thereof. In embodiments, the copolymer is a PLA-PEG di-block copolymer. In embodiments, the copolymer is a PLA-PEG-PLA tri-block copolymer. In embodiments, the copolymer is a PEG-PLA-PEG triblock copolymer. In embodiments, the copolymer is a PEG-PLA multi-block copolymer. In embodiments, the copolymer is a PLGA-PEG di-block copolymer. In embodiments, the copolymer is a PLGA-PEG-PLGA tri-block copolymer. In embodiments, the copolymer is a PEG-PLGA-PEG triblock copolymer. In embodiments, the copolymer is a PEG-PLGA multi-block copolymer. In embodiments, the copolymer is a PPG-PEG di-block copolymer. In embodiments, the copolymer is a PPG-PEG-PPG tri-block copolymer. In embodiments, the copolymer is a PEG-PPG-PEG triblock copolymer. In embodiments, the copolymer is a PEG-PPG multi-block copolymer. In embodiments, the copolymer is PCL-PEG di-block copolymer. In embodiments, the copolymer is a PCL-PEG-PCL tri-block copolymer. In embodiments, the copolymer is a PEG-PCL-PEG triblock copolymer. In embodiments, the copolymer is a PEG-PCL multi-block copolymer.

In aspects, the disclosure provides pharmaceutical composition comprising the polymeric compositions disclosed which are formulated to include therapeutic cells (such as, engineered cells (such as stem cells)) or a biological agent (such as, without limitation, an antibody or small molecule chemical compound) for delivery to a particular target tissue (such as, without limitation, neural tissue, heart tissue, bone tissue, liver tissue, kidney tissue, pancreas tissue, spleen tissue, bladder tissue, lung tissue, breast tissue, prostate tissue or cancerous tissue (e.g., tumors)) to produce a biological effect. In other embodiments, the pharmaceutical compositions and polymeric compositions disclosed herein can deliver therapeutic cells or biological agents to a lesion (for example, a brain tumor or any solid tumor) or a tissue surface (for example, the inner bladder) in order to induce a biological effect. Similarly, drug molecules (such as, without limitation, anticancer agents, antibiotics, nano or microparticles loaded with biological agents) can be delivered to the diseased site to induce an effective biological effect. The cell formulations should be in liquid form at the time of administration (for example, by injection using an 18-23G needle) and solidify upon contacting the target tissue.

In aspects, the polymer composition is a triblock copolymer made from PLA-PEG-PLA or PEG-PLGA-PE, where the PEG chain length and the PLA based blocks are selected to switch from an injectable solution to a semi-solid gel within seconds (such as within from about 1 to about 60 seconds; or about 1, 2, 3, 4, 5, 6, 7, 8, 9, 10, 15, 20, 25, or 30 seconds) of a temperature change taking place from room temperature (e.g., between about 0° C. to about 35° C.) to the physiological temperature upon injection (about 37° C.).

A limitation of some polymeric compositions have with respect to delivering therapeutic cells in this manner occurs when the cells become trapped within the gel and cannot proceed to the target tissue. To overcome this problem, a progressive degradable triblock copolymer can be utilized so that the triblock is divided into two water-soluble blocks that have substantially no gelling capacity and thus reduce the gel viscosity and allow cell movement towards tissue. In some embodiments, the reduction in viscosity of the polymeric composition can be designed by selecting a cleavable bond that either degrades by hydrolysis (such as, without limitation, anhydride bonds, glycolic-glycolic ester bond) or an enzymatically degradable bond, wherein the enzyme is secreted by the entrapped cells or by the cells of the target tissue. These possibilities are described in the following illustration.

PLA-PEG-PLA with fast degrading R labile bond

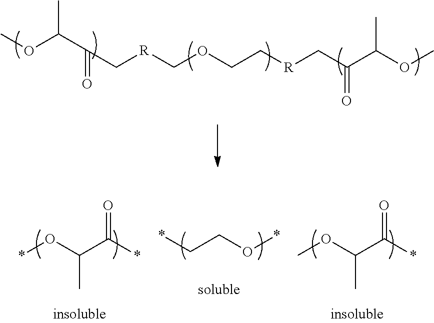

In embodiments, the polymeric compositions is a PLA-PEG-PLA triblock copolymer with cleavable bonds that form two water-soluble components, disrupting the gel. The triblock copolymer can optionally be described as PLA-R-PEG-R-PLA to identify the presence of the labile bond as R. In the non-limiting embodiment illustrated above, R is labile bond (or linker or linking group or linking moiety) that can be hydrolyzed by water or an enzyme (such as an enzyme secreted by the embedded therapeutic cells or an enzyme secreted by a target tissue (such as, a tumor)). The linker can be, without limitation, an anhydride bond, a short peptide that is cleavable by a proteases secreted by the cells (e.g., caspases or an ester bond that is cleaved by esterase). In other embodiments, the linker can be a photosensitive bond or pH sensitive bond that can be cleaved by an external trigger (such as, without limitation, light, pH, and ultrasound). The progress of cells towards the target tissue can dominated by the degradation of bonds within the gel, either by passive hydrolysis of susceptible bonds (anhydrides) or peptidases incorporated or released by the cells.

Labile Bonds

As discussed above, the non-crosslinked polymers disclosed herein possess a labile or cleavable bond or bonds that break upon application of an external trigger or by hydrolysis. For example, the cleavable bond can be bond that is cleaved by hydrolysis such as, without limitation, an anhydride bond or the cleavable bond can be an enzymatic cleavable bond by non-specific or specific protease or esterase enzymes. Bonds that degrade by oxidation or reduction such as —S—S— or —N═N— bonds are also appropriate for use in the polymer compositions disclosed herein as are bonds that change their configuration or cleave upon exposure to UV or visible light such as, without limitation, an ester of the photo cleavable groups o-nitrobenzyl, pyrene, or coumarin. In one embodiment, the cleavable polymeric composition can be illustrated by the following Scheme, using PLA-PEG-PLA as a representative polymer:

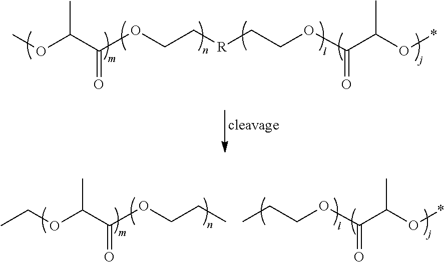

In the example shown above, the cleavable bond is within the PEG chain and upon cleavage by hydrolysis of enzymes, two non-gelling or less gelling fragments are formed which diminish the gel properties or change the gel to a more fluidic nature.

in embodiments, the labile bond is cleavable or susceptible to cleavage by hydrolysis (i.e., the cleavable bond is a water-cleavable bond which can be cleaved by the influence of and reaction with water). Non-limiting examples of hydrolytically susceptible bonds include an anhydride bond, a glycolic-glycolic ester bond, an imine bond, or a carboxyanhydride bond. In embodiments, the labile bond is susceptible to cleavage by rapid hydrolysis compared to other hydrolysable bonds in the block polymers. By “rapid hydrolysis” it is meant that the hydrolytically susceptible bond in the polymer is cleaved more rapidly than other hydrolytically susceptible bonds in the polymer, such as any of about 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 11×, 12×, 13×, 14×, 15×, 16×, 17×, 18×, 19×, 20×, 25×, 30×, 35×, 40×, 45×, 50×, 60×, 70×, 80×, 90×, or 100× more rapidly than other hydrolytically susceptible bonds in the polymer (inclusive of numbers falling within these values).

In embodiments, the polymer of the present disclosure includes a labile bond (for example, a peptide bond) that is cleavable or susceptible to cleavage by an enzyme, for example, a protease. A number of different enzymatically cleavable peptides may be used as labile bonds for the polymer of the present disclosure. In embodiments, the polymer includes a protease cleavable site which is cleaved at or near a tissue of interest such as, without limitation, in the small intestine, in a tumor microenvironment, or in neural tissue. The enzyme can be present at or near the tissue of interest or can be produced by cells of the tissue of interest (for example and without limitation, the enzyme can be produced by a neural cell, a bladder cell, a stem cell, a therapeutic cell, an engineered cell, a cancer cell, or an immune cell).

The enzyme can be a protease, such as, without limitation, a caspase, an esterase, a cysteine-protease, or a cathepsin. In embodiments, the enzymatically cleavable labile bond is a trypsin or chymotrypsin cleavable peptide or may be selectively cleavable by serine proteases, including trypsin and elastase, carboxypeptidases, or aminopeptidases. A wide variety of different protease cleavable peptides that comprise from 2 amino acid residues to 25 amino acid residues may be used as labile bonds in the polymers disclosed herein. For example the protease cleavable peptide can comprise from 5 amino acid residues to 15 amino acid residues, such as any of 5, 6, 7, 8, 9, 10, 11, 12, 13, 14, or 15 amino acid residues. In embodiments, the protease cleavable peptide comprises from 5 amino acid residues to 10 amino acid residues. In embodiments, the protease cleavable peptide comprises from 2 amino acid residues to 15 amino acid residues. In embodiments, the protease cleavable peptide comprises from 2 amino acid residues to 10 amino acid residues. Additionally, the protease cleavable peptide for use in any of the polymer compositions disclosed herein can include amino acid residues selected from lysine, arginine, or glycine.

The non-crosslinked polymers disclosed herein can further possess a labile bond that is cleavable or susceptible to cleavage by changes in pH. For example, the tumor microenvironment is often hypoxic. As tumor mass increases, the interior of the tumor grows farther away from existing blood supply leading to hypoxia. While a lack of oxygen can cause glycolytic behavior in cells, tumor cells also undergo aerobic glycolysis, in which they preferentially produce lactate from glucose even given abundant oxygen, called the Warburg effect. This leaves the extracellular microenvironment of tumors acidic (pH 6.5-6.9). In embodiments, a “pH-labile bond” refers to the selective breakage of a covalent bond under acidic conditions (pH <7, such as a pH of any of about 6.0, 6.1, 6.2, 6.3, 6.4, 6.5, 6.6, 6.7, 6.8, or 6.9). In embodiments, the pH-labile bond breaks at a pH from about 4 to about 6.9. In embodiments, the pH-labile bond breaks at a pH from about 5 to about 6.9. In embodiments, the pH-labile bond breaks at a pH from about 6 to about 6.9. That is, the pH-labile bond of any of the polymers disclosed herein may be broken under acidic conditions in the presence of other covalent bonds in the polymer without their breakage. The term pH-labile includes both linkages and bonds that are pH-labile, very pH-labile, and extremely pH-labile. A subset of pH-labile bonds is very pH-labile. A bond is considered very pH-labile if the half-life for cleavage at pH 5 is less than 45 minutes but more than 15 minutes (such as any of about 45, 40, 35, 30, 25, 20, 19, 18, 17, or 16 minutes). A further subset of pH-labile bonds is extremely pH-labile. A bond is considered extremely pH-labile if the half-life for cleavage at pH 5 is less than 15 minutes. Non-limiting examples of pH labile bonds include ketals that are labile in acidic environments (pH less than 7, greater than 4) to form a diol and a ketone, acetals that are labile in acidic environments (pH less than 7, greater than 4) to form a diol and an aldehyde and/or imines or iminiums that are labile in acidic environments (pH less than 7, greater than 4) to form an amine and an aldehyde or a ketone.

The polymers disclosed herein can also contain silicon-oxygen-carbon linkages that are labile under acidic conditions. Organosilanes have long been utilized as oxygen protecting groups in organic synthesis due to both the ease in preparation (of the silicon-oxygen-carbon linkage) and the facile removal of the protecting group under acidic conditions. For example, silyl ethers and silylenolethers, both possess such a linkage. Silicon-oxygen-carbon linkages are susceptible to hydrolysis under acidic conditions forming silanols and an alcohol (or enol). The substitution on both the silicon atom and the alcohol carbon can affect the rate of hydrolysis due to steric and electronic effects. This allows for the possibility of tuning the rate of hydrolysis of the silicon-oxygen-carbon linkage by changing the substitution on either the organosilane, the alcohol, or both the organosilane and alcohol to facilitate the desired effect. In addition, charged or reactive groups, such as amines or carboxylate, may be linked to the silicon atom, which confers the labile compound with charge and/or reactivity.

In embodiments, the polymer of the present disclosure includes a labile bond cleavable or susceptible to cleavage by photosensitization (i.e., the bonds have photosensitivity). Non-limiting examples of labile bonds cleavable or susceptible to cleavage by photosensitization include azobenzenes, triphenylmethanes leucohydroxides, nitrobenzyls, or cinnamates.

In embodiments, the polymer compositions are a copolymer of Formula I:

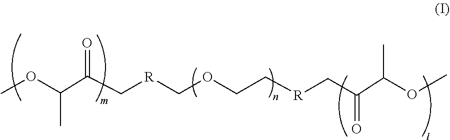

wherein m, n, and l are one or more polymeric subunits (e.g., from 1 to 1,000,000,000 polymeric subunits) and R is a labile bond. In embodiments, R is a physiologically labile bond. In embodiments, R is independently a labile bond cleavable by hydrolysis, an enzyme, photosensitization, a pH change, a soundwave, or a combination of two or more thereof. In embodiments, R is a labile bond cleavable by hydrolysis. In embodiments, R is a labile bond cleavable by rapid hydrolysis compared to other hydrolysable bonds in the block polymer. In embodiments, R is independently an anhydride bond, a glycolic-glycolic ester bond, an imine bond, or a carboxyanhydride bond. In embodiments, R is a labile bond cleavable by an enzyme. In embodiments, R is a labile bond cleavable by a protease. In embodiments, R is a labile bond cleavable by a caspase, an esterase, a cysteine-protease, or a cathepsin. In embodiments, R is a cleavable peptide bond comprising from 2 to 25 amino acid residues. In embodiments, R is a cleavable peptide bond comprising from 2 to 15 amino acid residues. In embodiments, R is a cleavable peptide bond comprising from 2 to 10 amino acid residues. In embodiments, R is a labile bond cleavable by a pH change. In embodiments, R is a labile bond cleavable by photosensitization. In embodiments, R is independently an azobenzene moiety, a triphenylmethane leucohydroxide moiety, a nitrobenzyl moiety, or a cinnamate moiety. In embodiments, R is independently —C(═O)—O—C(═O)—, —N═CH—, —C(═O)—CH₂—O—C(═O)—, or —S—S—. In embodiments, R is —C(═O)—O—C(═O)—. In embodiments, R is —N═CH—. In embodiments, R is —C(═O)—CH₂—O—C(═O)—. In embodiments, R is —S—S—. In embodiments, R is independently at least two of —C(═O)—O—C(═O)—, —N═CH—, —C(═O)—CH₂—O—C(═O)—, and —S—S—. In embodiments, n is a number that provides a polyethylene glycol having a number average molecular weight from about 100 Daltons to about 10,000 Daltons. In embodiments, n is a number that provides a polyethylene glycol having a number average molecular weight from about 100 Daltons to about 8,000 Daltons. In embodiments, n is a number that provides a polyethylene glycol having a number average molecular weight from about 500 Daltons to about 5,000 Daltons. In embodiments, n is a number that provides a polyethylene glycol having a number average molecular weight from about 1,000 Daltons to about 4,000 Daltons. In embodiments, n is a number that provides a polyethylene glycol having a number average molecular weight from about 1,500 Daltons to about 10,000 Daltons. In embodiments, n is a number that provides a polyethylene glycol having a number average molecular weight from about 2,000 Daltons to about 4,000 Daltons. In embodiments, n is a number that provides a polyethylene glycol having a number average molecular weight from about 1,500 Daltons to about 2,500 Daltons. In embodiments, n is a number that provides a polyethylene glycol having a number average molecular weight from about 3,000 Daltons to about 4,000 Daltons. In embodiments, n is a number that provides a polyethylene glycol having a number average molecular weight of about 2,000 Daltons. In embodiments, n is a number that provides a polyethylene glycol having a number average molecular weight of about 3,500 Daltons. In embodiments, m and l are numbers that provide a poly(lactic-co-glycolic acid) having a number average molecular weight from about 100 Daltons to about 10,000 Daltons. In embodiments, m and l are numbers that provide a poly(lactic-co-glycolic acid) having a number average molecular weight from about 500 Daltons to 5,000 Dalton. In embodiments, m and l are numbers that provide a poly(lactic-co-glycolic acid) having a number average molecular weight from about 1,000 Daltons to about 4,000 Daltons. In embodiments, m and l are numbers that provide a poly(lactic-co-glycolic acid) having a number average molecular weight from about 2,000 Daltons to about 4,000 Daltons. In embodiments, m and l are numbers that provide a poly(lactic-co-glycolic acid) having a number average molecular weight from about 2,000 Daltons to about 3,000 Daltons. In embodiments, m and l are numbers that provide a poly(lactic-co-glycolic acid) having a number average molecular weight from about 3,000 Daltons to about 4,000 Daltons. In embodiments, m and l are numbers that provide a poly(lactic-co-glycolic acid) having a number average molecular weight from about 3,500 Daltons to about 4,000 Daltons. In embodiments, the weight ratio of lactic acid to glycolic acid in the poly(lactic-co-glycolic acid) is from about 95:5 to about 5:95. In embodiments, the weight ratio of lactic acid to glycolic acid in the poly(lactic-co-glycolic acid) is from about 95:5 to about 50:50. In embodiments, the weight ratio of lactic acid to glycolic acid in the poly(lactic-co-glycolic acid) is from about 90:10 to about 50:50. In embodiments, the weight ratio of lactic acid to glycolic acid in the poly(lactic-co-glycolic acid) is from about 90:10 to about 60:40. In embodiments, the weight ratio of lactic acid to glycolic acid in the poly(lactic-co-glycolic acid) is from about 80:20 to about 50:50. In embodiments, the weight ratio of lactic acid to glycolic acid in the poly(lactic-co-glycolic acid) is from about 80:20 to about 60:40. In embodiments, the weight ratio of lactic acid to glycolic acid in the poly(lactic-co-glycolic acid) is about 50:50. In embodiments, the weight ratio of lactic acid to glycolic acid in the poly(lactic-co-glycolic acid) is about 60:40. In embodiments, the weight ratio of lactic acid to glycolic acid in the poly(lactic-co-glycolic acid) is about 65:35. in embodiments, the weight ratio of lactic acid to glycolic acid in the poly(lactic-co-glycolic acid) is about 70:30. In embodiments, the weight ratio of lactic acid to glycolic acid in the poly(lactic-co-glycolic acid) is about 75:25. In embodiments, the weight ratio of lactic acid to glycolic acid in the poly(lactic-co-glycolic acid) is about 80:20. In embodiments, the weight ratio of lactic acid to glycolic acid in the poly(lactic-co-glycolic acid) is about 85:15. In embodiments, the weight ratio of lactic acid to glycolic acid in the poly(lactic-co-glycolic acid) is about 90:10.

In aspects, the polymer compositions are a copolymer of Formula (II):

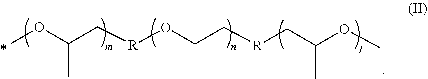

wherein m, n, and l are one or more polymeric subunits (e.g., from 1 to 1,000,000,000 polymeric subunits); R is a labile bond; and * is a terminal group. In embodiments, R is a physiologically labile bond. In embodiments, R is independently a labile bond cleavable by hydrolysis, an enzyme, photosensitization, a pH change, a soundwave, or a combination of two or more thereof. In embodiments, R is a labile bond cleavable by hydrolysis. In embodiments, R is a labile bond cleavable by rapid hydrolysis compared to other hydrolysable bonds in the block polymer. In embodiments, R is independently an anhydride bond, a glycolic-glycolic ester bond, an imine bond, or a carboxyanhydride bond. In embodiments, R is a labile bond cleavable by an enzyme. In embodiments, R is a labile bond cleavable by a protease. In embodiments, R is a labile bond cleavable by a caspase, an esterase, a cysteine-protease, or a cathepsin. In embodiments, R is a cleavable peptide bond comprising from 2 to 25 amino acid residues. In embodiments, R is a cleavable peptide bond comprising from 2 to 15 amino acid residues. In embodiments, R is a cleavable peptide bond comprising from 2 to 10 amino acid residues. In embodiments, R is a labile bond cleavable by a pH change. In embodiments, R is a labile bond cleavable by photosensitization. In embodiments, R is independently an azobenzene moiety, a triphenylmethane leucohydroxide moiety, a nitrobenzyl moiety, or a cinnamate moiety. In embodiments, R is independently —C(═O)—O—C(═O)—, —N═CH—, —C(═O)—CH₂—O—C(═O)—, or —S—S—. In embodiments, R is —C(═O)—O—C(═O)—. In embodiments, R is —N═CH—. In embodiments, R is —C(═O)—CH₂—O—C(═O)—. In embodiments, R is —S—S—. In embodiments, R is independently at least two of C(═O)—O—C(═O)—, —N═CH—, —C(═O)—CH₂—O—C(═O)—, and —S—S—. In embodiments, n is a number that provides a polyethylene glycol having a number average molecular weight from about 100 Daltons to about 10,000 Daltons. In embodiments, n is a number that provides a polyethylene glycol having a number average molecular weight from about 100 Daltons to about 8,000 Daltons. In embodiments, n is a number that provides a polyethylene glycol having a number average molecular weight from about 500 Daltons to about 5,000 Daltons. In embodiments, n is a number that provides a polyethylene glycol having a number average molecular weight from about 1,000 Daltons to about 4,000 Daltons. In embodiments, n is a number that provides a polyethylene glycol having a number average molecular weight from about 1,500 Daltons to about 10,000 Daltons. In embodiments, n is a number that provides a polyethylene glycol having a number average molecular weight from about 2,000 Daltons to about 4,000 Daltons. In embodiments, n is a number that provides a polyethylene glycol having a number average molecular weight from about 1,500 Daltons to about 2,500 Daltons. In embodiments, n is a number that provides a polyethylene glycol having a number average molecular weight from about 3,000 Daltons to about 4,000 Daltons. In embodiments, n is a number that provides a polyethylene glycol having a number average molecular weight of about 2,000 Daltons. In embodiments, n is a number that provides a polyethylene glycol having a number average molecular weight of about 3,500 Daltons. In embodiments, m and l are numbers that provide a polypropylene glycol having a number average molecular weight from about 100 Daltons to about 10,000 Daltons. In embodiments, m and l are numbers that provide a polypropylene glycol having a number average molecular weight from about 500 Daltons to 5,000 Dalton. In embodiments, m and l are numbers that provide a polypropylene glycol having a number average molecular weight from about 1,000 Daltons to about 4,000 Daltons. In embodiments, m and l are numbers that provide a polypropylene glycol having a number average molecular weight from about 1,000 Daltons to about 3,000 Daltons. In embodiments, m and l are numbers that provide a polypropylene glycol having a number average molecular weight from about 1,000 Daltons to about 2,000 Daltons. In embodiments, m and l are numbers that provide a polypropylene glycol having a number average molecular weight from about 2,000 Daltons to about 4,000 Daltons. In embodiments, m and l are numbers that provide a polypropylene glycol having a number average molecular weight from about 2,000 Daltons to about 3,000 Daltons. In embodiments, m and l are numbers that provide a polypropylene glycol having a number average molecular weight from about 3,000 Daltons to about 4,000 Daltons.

In aspects, the polymer compositions are a copolymer of Formula (III):

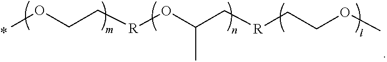

wherein m, n, and l are one or more polymeric subunits (e.g., from 1 to 1,000,000,000 polymeric subunits); R is a labile bond; and * is a terminal group. In embodiments, R is a physiologically labile bond. In embodiments, R is independently a labile bond cleavable by hydrolysis, an enzyme, photosensitization, a pH change, a soundwave, or a combination of two or more thereof. In embodiments, R is a labile bond cleavable by hydrolysis. In embodiments, R is a labile bond cleavable by rapid hydrolysis compared to other hydrolysable bonds in the block polymer. In embodiments, R is independently an anhydride bond, a glycolic-glycolic ester bond, an imine bond, or a carboxyanhydride bond. In embodiments, R is a labile bond cleavable by an enzyme. In embodiments, R is a labile bond cleavable by a protease. In embodiments, R is a labile bond cleavable by a caspase, an esterase, a cysteine-protease, or a cathepsin. In embodiments, R is a cleavable peptide bond comprising from 2 to 25 amino acid residues. In embodiments, R is a cleavable peptide bond comprising from 2 to 15 amino acid residues. In embodiments, R is a cleavable peptide bond comprising from 2 to 10 amino acid residues. In embodiments, R is a labile bond cleavable by a pH change. In embodiments, R is a labile bond cleavable by photosensitization. In embodiments, R is independently an azobenzene moiety, a triphenylmethane leucohydroxide moiety, a nitrobenzyl moiety, or a cinnamate moiety. In embodiments, R is independently —C(═O)—O—C(═O)—, —N═CH—, —C(═O)—CH₂—O—C(═O)—, or —S—S—. In embodiments, R is —C(═O)—O—C(═O)—. In embodiments, R is —N═CH—. In embodiments, R is —C(═O)—CH₂—O—C(═O)—. In embodiments, R is —S—S—. In embodiments, R is independently at least two of —C(═O)—O—C(═O)—, —N═CH—, —C(═O)—CH₂—O—C(═O)—, and —S—S—. In embodiments, m and l are numbers that provide a polyethylene glycol having a number average molecular weight from about 100 Daltons to about 10,000 Daltons. In embodiments, m and l are numbers that provide a polyethylene glycol having a number average molecular weight from about 100 Daltons to about 8,000 Daltons. In embodiments, m and l are numbers that provide a polyethylene glycol having a number average molecular weight from about 500 Daltons to about 5,000 Daltons. In embodiments, m and l are numbers that provide a polyethylene glycol having a number average molecular weight from about 1,000 Daltons to about 4,000 Daltons. In embodiments, m and l are numbers that provide a polyethylene glycol having a number average molecular weight from about 1,500 Daltons to about 10,000 Daltons. In embodiments, m and l are numbers that provide a polyethylene glycol having a number average molecular weight from about 2,000 Daltons to about 4,000 Daltons. In embodiments, m and l are numbers that provide a polyethylene glycol having a number average molecular weight from about 1,500 Daltons to about 2,500 Daltons. In embodiments, m and l are numbers that provide a polyethylene glycol having a number average molecular weight from about 3,000 Daltons to about 4,000 Daltons. In embodiments, m and l are numbers that provide a polyethylene glycol having a number average molecular weight of about 2,000 Daltons. In embodiments, m and l are numbers that provide a polyethylene glycol having a number average molecular weight of about 3,500 Daltons. In embodiments, n is a number that provides a polypropylene glycol having a number average molecular weight from about 100 Daltons to about 10,000 Daltons. In embodiments, n is a number that provides a polypropylene glycol having a number average molecular weight from about 500 Daltons to 5,000 Dalton. In embodiments, n is a number that provides a polypropylene glycol having a number average molecular weight from about 1,000 Daltons to about 4,000 Daltons. In embodiments, n is a number that provides a polypropylene glycol having a number average molecular weight from about 1,000 Daltons to about 3,000 Daltons. In embodiments, n is a number that provides a polypropylene glycol having a number average molecular weight from about 1,000 Daltons to about 2,000 Daltons. In embodiments, n is a number that provides a polypropylene glycol having a number average molecular weight from about 2,000 Daltons to about 4,000 Daltons. In embodiments, n is a number that provides a polypropylene glycol having a number average molecular weight from about 2,000 Daltons to about 3,000 Daltons. In embodiments, n is a number that provides a polypropylene glycol having a number average molecular weight from about 3,000 Daltons to about 4,000 Daltons.

In aspects, the polymer compositions are a copolymer of Formula (IV):

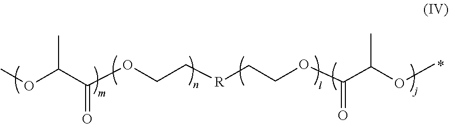

wherein m, n, l, and j are one or more polymeric subunits (e.g., from 1 to 1,000,000,000 polymeric subunits); R is a labile bond; and * is a terminal group. In embodiments, R is a physiologically labile bond. In embodiments, R is a labile bond cleavable by hydrolysis, an enzyme, photosensitization, a pH change, a soundwave, or a combination of two or more thereof. In embodiments, R is a labile bond cleavable by hydrolysis. In embodiments, R is a labile bond cleavable by rapid hydrolysis compared to other hydrolysable bonds in the block polymer. In embodiments, R is an anhydride bond, a glycolic-glycolic ester bond, an imine bond, or a carboxyanhydride bond. In embodiments, R is a labile bond cleavable by an enzyme. In embodiments, R is a labile bond cleavable by a protease. In embodiments, R is a labile bond cleavable by a caspase, an esterase, a cysteine-protease, or a cathepsin. In embodiments, R is a cleavable peptide bond comprising from 2 to 25 amino acid residues. In embodiments, R is a cleavable peptide bond comprising from 2 to 15 amino acid residues. In embodiments, R is a cleavable peptide bond comprising from 2 to 10 amino acid residues. In embodiments, R is a labile bond cleavable by a pH change. In embodiments, R is a labile bond cleavable by photosensitization. In embodiments, R is an azobenzene moiety, a triphenylmethane leucohydroxide moiety, a nitrobenzyl moiety, or a cinnamate moiety. In embodiments, R is —C(═O)—O—C(═O)—, —N═CH—, —C(═O)—CH₂—O—C(═O)—, or —S—S—. In embodiments, R is —C(═O)—O—C(═O)—. In embodiments, R is —N═CH—. In embodiments, R is —C(═O)—CH₂—O—C(═O)—. In embodiments, R is —S—S—. In embodiments, n and 1 are numbers that provide a polyethylene glycol having a number average molecular weight from about 100 Daltons to about 10,000 Daltons. In embodiments, n and 1 are numbers that provide a polyethylene glycol having a number average molecular weight from about 100 Daltons to about 8,000 Daltons. In embodiments, n and 1 are numbers that provide a polyethylene glycol having a number average molecular weight from about 500 Daltons to about 5,000 Daltons. In embodiments, n and 1 are numbers that provide a polyethylene glycol having a number average molecular weight from about 1,000 Daltons to about 4,000 Daltons. In embodiments, n and 1 are numbers that provide a polyethylene glycol having a number average molecular weight from about 1,500 Daltons to about 10,000 Daltons. In embodiments, n and 1 are numbers that provide a polyethylene glycol having a number average molecular weight from about 2,000 Daltons to about 4,000 Daltons. In embodiments, n and 1 are numbers that provide a polyethylene glycol having a number average molecular weight from about 1,500 Daltons to about 2,500 Daltons. In embodiments, n and 1 are numbers that provide a polyethylene glycol having a number average molecular weight from about 3,000 Daltons to about 4,000 Daltons. In embodiments, n and 1 are numbers that provide a polyethylene glycol having a number average molecular weight of about 2,000 Daltons. In embodiments, n and 1 are numbers that provide a polyethylene glycol having a number average molecular weight of about 3,500 Daltons. In embodiments, m and j are numbers that provide a poly(lactic-co-glycolic acid) having a number average molecular weight from about 100 Daltons to about 10,000 Daltons. In embodiments, m and j are numbers that provide a poly(lactic-co-glycolic acid) having a number average molecular weight from about 500 Daltons to 5,000 Dalton. In embodiments, m and j are numbers that provide a poly(lactic-co-glycolic acid) having a number average molecular weight from about 1,000 Daltons to about 4,000 Daltons. In embodiments, m and j are numbers that provide a poly(lactic-co-glycolic acid) having a number average molecular weight from about 2,000 Daltons to about 4,000 Daltons. In embodiments, m and j are numbers that provide a poly(lactic-co-glycolic acid) having a number average molecular weight from about 2,000 Daltons to about 3,000 Daltons. In embodiments, m and j are numbers that provide a poly(lactic-co-glycolic acid) having a number average molecular weight from about 3,000 Daltons to about 4,000 Daltons. In embodiments, m and j are numbers that provide a poly(lactic-co-glycolic acid) having a number average molecular weight from about 3,500 Daltons to about 4,000 Daltons. In embodiments, the weight ratio of lactic acid to glycolic acid in the poly(lactic-co-glycolic acid) is from about 95:5 to about 5:95. In embodiments, the weight ratio of lactic acid to glycolic acid in the poly(lactic-co-glycolic acid) is from about 95:5 to about 50:50. In embodiments, the weight ratio of lactic acid to glycolic acid in the poly(lactic-co-glycolic acid) is from about 90:10 to about 50:50. In embodiments, the weight ratio of lactic acid to glycolic acid in the poly(lactic-co-glycolic acid) is from about 90:10 to about 60:40. In embodiments, the weight ratio of lactic acid to glycolic acid in the poly(lactic-co-glycolic acid) is from about 80:20 to about 50:50. In embodiments, the weight ratio of lactic acid to glycolic acid in the poly(lactic-co-glycolic acid) is from about 80:20 to about 60:40. In embodiments, the weight ratio of lactic acid to glycolic acid in the poly(lactic-co-glycolic acid) is about 50:50. In embodiments, the weight ratio of lactic acid to glycolic acid in the poly(lactic-co-glycolic acid) is about 60:40. In embodiments, the weight ratio of lactic acid to glycolic acid in the poly(lactic-co-glycolic acid) is about 65:35. In embodiments, the weight ratio of lactic acid to glycolic acid in the poly(lactic-co-glycolic acid) is about 70:30. In embodiments, the weight ratio of lactic acid to glycolic acid in the poly(lactic-co-glycolic acid) is about 75:25. In embodiments, the weight ratio of lactic acid to glycolic acid in the poly(lactic-co-glycolic acid) is about 80:20. In embodiments, the weight ratio of lactic acid to glycolic acid in the poly(lactic-co-glycolic acid) is about 85:15. In embodiments, the weight ratio of lactic acid to glycolic acid in the poly(lactic-co-glycolic acid) is about 90:10.

In aspects, the polymer compositions are a copolymer of Formula (V):

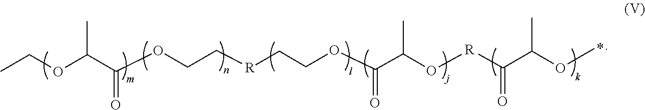

wherein m, n, l, j, and k are one or more polymeric subunits (e.g., from 1 to 1,000,000,000 polymeric subunits); R is a labile bond; and * is a terminal group. In embodiments, R is a physiologically labile bond. In embodiments, R is independently a labile bond cleavable by hydrolysis, an enzyme, photosensitization, a pH change, a soundwave, or a combination of two or more thereof. In embodiments, R is a labile bond cleavable by hydrolysis. In embodiments, R is a labile bond cleavable by rapid hydrolysis compared to other hydrolysable bonds in the block polymer. In embodiments, R is independently an anhydride bond, a glycolic-glycolic ester bond, an imine bond, or a carboxyanhydride bond. In embodiments, R is a labile bond cleavable by an enzyme. In embodiments, R is a labile bond cleavable by a protease. In embodiments, R is a labile bond cleavable by a caspase, an esterase, a cysteine-protease, or a cathepsin. In embodiments, R is a cleavable peptide bond comprising from 2 to 25 amino acid residues. In embodiments, R is a cleavable peptide bond comprising from 2 to 15 amino acid residues. In embodiments, R is a cleavable peptide bond comprising from 2 to 10 amino acid residues. In embodiments, R is a labile bond cleavable by a pH change. In embodiments, R is a labile bond cleavable by photosensitization. In embodiments, R is independently an azobenzene moiety, a triphenylmethane leucohydroxide moiety, a nitrobenzyl moiety, or a cinnamate moiety. In embodiments, R is independently —C(═O)—O—C(═O)—, —N═CH—, —C(═O)—CH₂—O—C(═O)-, or —S—S—. In embodiments, R is —C(═O)—O—C(═O)—. In embodiments, R is —N═CH—. In embodiments, R is —C(═O)—CH₂—O—C(═O)—. In embodiments, R is —S—S—. In embodiments, R is independently at least two of —C(═O)—O—C(═O)—, —N═CH—, —C(═O)—CH₂—O—C(═O)—, and —S—S—. In embodiments, n and 1 are numbers that provide a polyethylene glycol having a number average molecular weight from about 100 Daltons to about 10,000 Daltons. In embodiments, n and 1 are numbers that provide a polyethylene glycol having a number average molecular weight from about 100 Daltons to about 8,000 Daltons. In embodiments, n and 1 are numbers that provide a polyethylene glycol having a number average molecular weight from about 500 Daltons to about 5,000 Daltons. In embodiments, n and 1 are numbers that provide a polyethylene glycol having a number average molecular weight from about 1,000 Daltons to about 4,000 Daltons. In embodiments, n and 1 are numbers that provide a polyethylene glycol having a number average molecular weight from about 1,500 Daltons to about 10,000 Daltons. In embodiments, n and 1 are numbers that provide a polyethylene glycol having a number average molecular weight from about 2,000 Daltons to about 4,000 Daltons. In embodiments, n and 1 are numbers that provide a polyethylene glycol having a number average molecular weight from about 1,500 Daltons to about 2,500 Daltons. In embodiments, n and 1 are numbers that provide a polyethylene glycol having a number average molecular weight from about 3,000 Daltons to about 4,000 Daltons. In embodiments, n and 1 are numbers that provide a polyethylene glycol having a number average molecular weight of about 2,000 Daltons. In embodiments, n and 1 are numbers that provide a polyethylene glycol having a number average molecular weight of about 3,500 Daltons. In embodiments, m, j and k are numbers that provide a poly(lactic-co-glycolic acid) having a number average molecular weight from about 100 Daltons to about 10,000 Daltons. In embodiments, m, j and k are numbers that provide a poly(lactic-co-glycolic acid) having a number average molecular weight from about 500 Daltons to 5,000 Dalton. In embodiments, m, j and k are numbers that provide a poly(lactic-co-glycolic acid) having a number average molecular weight from about 1,000 Daltons to about 4,000 Daltons. In embodiments, m, j and k are numbers that provide a poly(lactic-co-glycolic acid) having a number average molecular weight from about 2,000 Daltons to about 4,000 Daltons. In embodiments, m, j and k are numbers that provide a poly(lactic-co-glycolic acid) having a number average molecular weight from about 2,000 Daltons to about 3,000 Daltons. In embodiments, m, j and k are numbers that provide a poly(lactic-co-glycolic acid) having a number average molecular weight from about 3,000 Daltons to about 4,000 Daltons. In embodiments, m, j and k are numbers that provide a poly(lactic-co-glycolic acid) having a number average molecular weight from about 3,500 Daltons to about 4,000 Daltons. In embodiments, the weight ratio of lactic acid to glycolic acid in the poly(lactic-co-glycolic acid) is from about 95:5 to about 5:95. in embodiments, the weight ratio of lactic acid to glycolic acid in the poly(lactic-co-glycolic acid) is from about 95:5 to about 50:50. In embodiments, the weight ratio of lactic acid to glycolic acid in the poly(lactic-co-glycolic acid) is from about 90:10 to about 50:50. In embodiments, the weight ratio of lactic acid to glycolic acid in the poly(lactic-co-glycolic acid) is from about 90:10 to about 60:40. In embodiments, the weight ratio of lactic acid to glycolic acid in the poly(lactic-co-glycolic acid) is from about 80:20 to about 50:50. In embodiments, the weight ratio of lactic acid to glycolic acid in the poly(lactic-co-glycolic acid) is from about 80:20 to about 60:40. In embodiments, the weight ratio of lactic acid to glycolic acid in the poly(lactic-co-glycolic acid) is about 50:50. In embodiments, the weight ratio of lactic acid to glycolic acid in the poly(lactic-co-glycolic acid) is about 60:40. In embodiments, the weight ratio of lactic acid to glycolic acid in the poly(lactic-co-glycolic acid) is about 65:35. In embodiments, the weight ratio of lactic acid to glycolic acid in the poly(lactic-co-glycolic acid) is about 70:30. In embodiments, the weight ratio of lactic acid to glycolic acid in the poly(lactic-co-glycolic acid) is about 75:25 In embodiments, the weight ratio of lactic acid to glycolic acid in the poly(lactic-co-glycolic acid) is about 80:20. In embodiments, the weight ratio of lactic acid to glycolic acid in the poly(lactic-co-glycolic acid) is about 85:15. hi embodiments, the weight ratio of lactic acid to glycolic acid in the poly(lactic-co-glycolic acid) is about 90:10.

The “terminal group” or “*” in the compounds of Formula (II), Formula (III), Formula (IV), and Formula (V) is a chemical moiety that forms the terminus of a polymer and can be any group known in the art. Example terminal groups include chemical groups (e.g., amine, thiol, hydroxyl, azide, carboxyl); protecting groups; polymerizable groups (e.g., acrylate, methacrylate, alkene); reactive groups (e.g., NHS, maleimide, isocyanate, vinyl sulphone); biotin; fluorophores; contrast agents; radio isotopes; enzymes (e.g., proteases (e.g., cathepsin B, CAPs, PSA), lipases (e.g., PLA2), glycosidases (e.g., amilase), urease, glucose oxidase, peroxidase, esterase, amidase); cell-penetrating peptides; targeting peptides; targeting antibodies; intracellular localization signals; anti-microbial peptides; poly(NIPAM-acrylamide); poly(NIPAM-vinylpyrrolidone); poly(methylvinylether); poly(N-vinylcaprolactam); gold nanoshell; nanorods; silver; titanium dioxide; fullerene; gold; zinc oxide; polyethylene glycol; gelatin; dextran; collagen; glucose; galactose; fructose; glucoronic acid; xylose; mannose; DNA; RNA (e.g., dsRNA, siRNA, shrRNA, crRNA); peptide nucleic acid; glycol nucleic acid; acetyl; phthalyl; methoxy; hydroxpropoxy; succinyl; carboxymethyl; imine; amino ester; ketal; polyhistidine; hydrazone; hydrazide; oxime; acetal; dimethyl maleate; disulfide; azobenzene; nitroaromatic; and quinone. In embodiments, the terminal group is hydrogen, —OH, —COOH, —CONH₂, —COH, —C(O)CH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the terminal group is hydrogen, —OH, —CH₃, —COOH, —CONH₂, —COH, or —C(O)CH₂. In embodiments, the terminal group is hydrogen. In embodiments, the terminal group is —OH. In embodiments, the terminal group is —CH₃. In embodiments, the terminal group is —COOH. In embodiments, the terminal group is —CONH₂. In embodiments, the terminal group is —COH. In embodiments, the terminal group is —C(O)CH₂.

For any of the polymer compositions disclosed herein, labile bond cleavage at physiological temperatures can result in degradation of the gel into a mixture of water-soluble to water insoluble components. In embodiments, the water insoluble components of the degraded gel outnumber the water-soluble components from about 10:1 to about 1.1:1. In embodiments, the water insoluble components of the degraded gel outnumber the water-soluble components from about 10:1 to about 1.5:1. In embodiments, the water insoluble components of the degraded gel outnumber the water-soluble components from about 4:1 to about 2:1. In embodiments, the water insoluble components of the degraded gel outnumber the water-soluble components by about 2:1.

In embodiments, the polymer compositions disclosed herein can be adapted for use as a bio-ink. Bio-inks are solutions containing biological elements such as cells, peptides and proteins, nucleic acid active agents and other active agents, that are used for 3D printing of matrices that contain different biologics (e.g., cells) distributed throughout the matrix in a planned manner. The ink must solidify at the spot of printing, induced by light or change in temperature (Hospodiuk et al., The bioink: A comprehensive review on bioprintable materials, Biotechnology Advances, 2017, 35(2):217-39). The polymers disclosed herein are thermoresponsive and thus solidify into a gel upon change in temperature. Cell printing is a new tool for better design scaffolds for tissue engineering, regenerative medicine and cell therapy. These three dimensional scaffolds can provide highly heterogeneous biological structures that better mimic natural tissues. To allow the design of diverse complex scaffolds with multiple functionalities and cell type, location in the scaffold and composition as well as nutrients and controlling agents such as growth factors, polymers of different properties that can be manipulated after printing are required.

With respect to bio-inks and printing of bio-inks, the thermoresponsive polymeric compositions disclosed herein can be manipulated after printing to meet various scaffold needs. At the time of printing, the polymers disclosed herein form a low viscosity solution in water. Therefore, additional components such as cells, nutrients, growth factors, and/or drugs can be added to the solution used as a printing bionic wherein the printed dot solidify as a function of temperature change. The printed scaffold can be further manipulated, depending on the type of cleavable bond (such as any of the cleavable or labile bonds disclosed herein) inserted in the critical gelling block and between blocks. For example, if a certain site at the 3D printed scaffold needs to become less viscose or even liquefied, a fast hydrolytically degrading bond can be added among the blocks so that at a certain time after fabrication the bond will be cleaved and result in the degradation of the gel at this spot into a solution. Several types of dual responsive inks can be prepared, for example, one that responds to fast hydrolysis and contains anhydride or imine bonds among the blocks, one that has S—S bonds that collapses as a response to reduction, one that degrades in the presence of a certain enzyme, and/or one that degrades as a function of exposure to light sources or specific wavelengths of light or other sources of electromagnetic radiation.

Therapeutic Agents for Inclusion in Polymeric Compositions

The disclosure provides pharmaceutical compositions comprising a therapeutic agent and the polymer compositions described herein. In embodiments, the therapeutic agent is embedded or included into the gel for administration into an individual. In another embodiment, any of the polymeric compositions disclosed herein can comprise one or more therapeutic agents. As used herein, a “therapeutic agent” or “therapeutic” refers to one or more substances that contribute to or causes the eradication of a disease state by, for example, killing an organism (such as a virus or pathogenic microorganism) or by control of erratic or harmful cellular growth or expression. The therapeutic agent can be an antibody, a functional fragment of an antibody, a small molecule chemical compound, a nucleic acid (e.g., a therapeutic nucleic acid or an inhibitory nucleic acid), a polypeptide, a nanoparticle, a contrasting agent, or a cell (such as an engineered cell).

In embodiments, the pharmaceutical compositions and polymer compositions described herein may comprise or be used to deliver one or more therapeutic nucleic acids or polynucleotides. The therapeutic nucleic acid or polynucleotide may be an inhibitory nucleic acid that can reduce the expression or translation of a gene or promote degradation of particular RNA species. In embodiments, the therapeutic nucleic acid may cause or promote the transcription or activation of a gene or gene product. For example, in embodiments the therapeutic nucleic acid may comprise a promoter operably linked to a polynucleotide that encodes a therapeutic protein: optionally, the nucleic acid may also encode an enhancer. In embodiments, the therapeutic nucleic acid is from 15-50, 17-30, or 17-25 nucleotides in length, or any range derivable therein. Examples of an inhibitory nucleic acid that may be used include but are not limited to molecules targeted to an nucleic acid sequence, such as an small interfering RNA (siRNA), short hairpin RNA (shRNA), double-stranded RNA, micro RNA (miRNA) an antisense oligonucleotide, a ribozyme and molecules targeted to a gene or gene product such as an aptamer.

An inhibitory nucleic acid may selectively inhibit the transcription of a gene or prevent the translation of the gene transcript in a cell. An inhibitory nucleic acid may be, e.g., from 4-1000 or 16-1000 nucleotides long. In embodiments, an inhibitory nucleic acid is from 18 to 100 nucleotides long. Various therapeutic nucleotides are known in the art. For example, genes that may be therapeutically targeted by a nucleic acid include, e.g., tumor necrosis factor-α. In some embodiments, the therapeutic nucleic acid may be transcribed in a cell to produce a therapeutic protein in the cell. Inhibitory nucleic acids are well known m the art. For example, siRNA, shRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

In designing a nucleic acid capable of generating an RNAi effect, there are several factors that need to be considered such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Particularly the siRNA exhibits greater than 80, 85, 90, 95, 98% or even 100% identity or complementarity between the sequence of the siRNA and a portion of an target nucleotide sequence. Sequences less than about 80% identical to the target gene are typically substantially less effective. Thus, the greater identity between the siRNA and the gene to be inhibited, the less likely expression of unrelated genes will be affected.

In addition, the size of the siRNA may be an important consideration. In some embodiments, siRNA molecules that are from 19-27 nucleotides in length, more preferably 20-25 nucleotides in length, may be used as the therapeutic nucleotide and may be used to selectively inhibit translation of a particular gene. In some embodiments, the therapeutic nucleotide is an antisense oligonucleotide. The antisense oligonucleotide may be less than 500, 200, 100, 50, 25, or 20 nucleotides in length. In some embodiments, the therapeutic nucleotide is an miRNA that is from about 19-24, or 19, 20, 21, 22, 23 nucleotides in length, or any range derivable therein.

Within an inhibitory nucleic acid, the components of a nucleic acid need not he of the same type or homogenous throughout (e.g., an inhibitory nucleic acid may comprise a nucleotide and a nucleic acid or nucleotide analog). Typically, an inhibitory nucleic acid can form a double-stranded structure; the double-stranded structure may result from two separate nucleic acids that are partially or completely complementary. In certain embodiments of the present invention, the inhibitory nucleic acid may comprise only a single nucleic acid (polynucleotide) or nucleic acid analog and form a double-stranded structure by complementing with itself (e.g., forming a hairpin loop). The double-stranded structure of the inhibitory nucleic acid may comprise 16-500 or more contiguous nucleobases, including all ranges derivable thereof. The inhibitory nucleic acid may comprise or consist of 17 to 35 contiguous nucleobases, more particularly 18 to 30 contiguous nucleobases, more particularly 19 to 25 nucleobases, more particularly 20 to 23 contiguous nucleobases, or 20 to 22 contiguous nucleobases, or 21 contiguous nucleobases that can selectively hybridize with a complementary nucleic acid within the same sequence or with a separate mRNA of interest (e.g., the complementary sequence may be located on the same nucleic acid or may be present in a separate complementary nucleic acid) to form a double-stranded structure. In some embodiments, the RNA may be protected with a chemical modification to slow degradation in the body or bloodstream of a mammalian subject such as a human. In some embodiments, the therapeutic RNA is a locked nucleic acid (LNA).

siRNA can be obtained from commercial sources, natural sources, or can be synthesized using any of a number of techniques well-known to those of ordinary skill in the art. An inhibitory nucleic acid that can be applied in the compositions and methods of the present invention may be any nucleic acid sequence that has been found by any source to be a validated downregulator of the gene or gene product. In one embodiment, the siRNA molecule is at least 75, 80, 85, or 90% homologous, particularly at least 95%, 99%, or 100% similar or identical, or any percentages in between the foregoing (e.g., the invention contemplates 75% and greater, 80% and greater, 85% and greater, and so on, and the ranges are intended to include all whole numbers in between), to at least 10 contiguous nucleotides of any of the nucleic acid sequences encoding a target therapeutic protein.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain embodiments, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Publication 2004/0019001 and U.S. Pat. No. 6,673,611 (each of which is incorporated by reference in its entirety). Collectively, all such altered nucleic acids or RNAs described above are referred to as modified siRNAs.

In embodiments, siRNA is capable of decreasing the expression of a particular genetic product by at least 10%, at least 20%, at least 30%, or at least 40%, at least 50%, at least 60%, or at least 70%, at least 75%, at least 80%, at least 90%, at least 95% or more or any ranges in between the foregoing.

In embodiments, the pharmaceutical compositions and polymer compositions disclosed herein may comprise or contain a therapeutic protein. The therapeutic protein may be a natural and nonnatural (e.g., recombinant) proteins, polypeptides, and peptides. In addition to proteins, the hydrogel network also may include polysaccharides, and particularly mixtures of mucopolysaccharides, carbohydrates, lipids; other organic compounds. For therapeutic applications, the protein may be biologically active.

Examples of therapeutic proteins that may be used in conjunction with the pharmaceutical compositions and polymeric compositions disclosed herein include, but are not limited to, synthetic, natural, or recombinant sources of: a growth hormone (e.g., a somatotropin, e.g., GENOTROPIN®, NUTROPIN®, NORDIROPIN®, SAIZEN®, SEROSTIM®, or HUMATROPE®), including a human growth hormone (hGH), a recombinant human growth hormone (rhGH), a bovine growth hormone, or a porcine growth hormone; a growth hormone-releasing hormone; an interferon (e.g., IFN-γ, IFN-α, IFN-β, IFN-τ; IFN-κ); an interleukin IL-I; IL-2, including, e.g., PROLEUKTN®; IL-3, IL-4, IL-5, IL-6, IL-7, IL-8, IL-9; and the like); a growth factor (e.g., REGRANEX® (beclapermin; PDGF); FIBLAST® (trafermin; bFGF); STEMGEN® (ancestim; stem cell factor); a keratinocyte growth factor; an acidic fibroblast growth factor, a stem cell factor, a basic fibroblast growth factor, a hepatocyte growth factor; insulin, including porcine, bovine, human, and human recombinant insulin Novolin, Humulin, Humalog, Lantus, Ultralente), optionally having counter ions including sodium, zinc, calcium and ammonium; an insulin-like growth factor, including IGF-1; a heparin, including unfractionated heparin, heparinoids, dermatans, chondroitins, low molecular weight heparin, very low molecular weight heparin and ultra low molecular weight heparin; calcitonin, including salmon, eel, and human calcitonin; erythropoietin (e.g., PROCRIT®, EPREX®, or EPOGEN® (epoetin-α); ARANESP® (darbepoetin-α); NEORECORMON®, EPOGIN®) (epoetin-β); and thelike); a blood factor (e.g., ACTIVASE® (alteplase) tissue plasminogen activator; NOVOSEVEN® (recombinant human factor VIIa); Factor VIIa; Factor VIII (e.g., KOGENATE®); Factor IX; β-globin; hemoglobin; and the like); a colony stimulating factor (e.g., NEUPOGEN® (filgrastim; G-CSF), NEULASTA® (pegfilgrastim), a granulocyte colony stimulating factor (G-CSF), a granulocyte-monocyte colony stimulating factor, a macrophage colony stimulating factor, a megakaryocyte colony stimulating factor; and the like); an antigen; an antibody (e.g, a monoclonal antibody) (e.g., RITUXAN® (rituximab); REMICADE® (infliximab); HERCEPTIN® (trastuzumab); HUIIVIIRA™ (adalimumab); XOLAIR® (omalizumab); BEXXAR® (tositumomab); RAPTIVA™ (efalizumab); ERBITUX™ (cetuximab); and the like), an scFv region, or an antibody fragment, including an antigen-binding fragment of a monoclonal antibody; a soluble receptor (e.g., a TNF-α-binding soluble receptor such as ENBREL® (etanercept); a soluble VEGF receptor; a soluble interleukin receptor; a soluble γ/δ T cell receptor; and the like); an enzyme (e.g., α-glucosidase; CERAZYME® (imiglucarase; 3-glucocerebrosidase, CEREDASE® (alglucerase); an enzyme activator (e.g., tissue plasminogen activator); a chemokine (e.g., IP-IP; Mig; Groα/IL-8, RANTES; MIP-Ia; MIP-Iβ; MCP-I; PF-4; and the like); an angiogenic agent (e.g., vascular endothelial growth factor (VEGF); an anti-angiogenic agent (e.g., a soluble VEGF receptor); a neuroactive peptide such as bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, warfarin, dynorphin, neurotensin, motilin, thyrotropin, neuropeptide Y, luteinizing hormone, calcitonin, insulin, glucagon, vasopressin, angiotensin II, thyrotropin-releasing hormone, vasoactive intestinal peptide, a sleep peptide, nesiritide, octreotide, teriparatide, pramlintide, and the like; a thrombolytic agent; an atrial natriuretic peptide; a bone morphogenic protein; thrombopoietin; relaxin; glial fibrillary acidic protein; a follicle stimulating hormone; a human alpha-1 antitrypsin; a leukemia inhibitory factor; a transforming growth factor; a tissue factor; a luteinizing hormone; a leutinizing-hormone-releasing-hormone; a macrophage activating factor, a tumor necrosis factor, a neutrophil chemotactic factor, a nerve growth factor, a tissue inhibitor of metalloproteinases; a vasoactive intestinal peptide, angiogenin, angiotropin, fibrin; hirudin; a leukemia inhibitory factor; an IL-I receptor antagonist (e.g., KINERET® (anakinra)); a protease inhibitor; adrenocorticotropin; a prostaglandin; cyclosporin; cromolyn sodium (sodium or disodium chromoglycate); vancomycin; desferoxamine (DFO); parathyroid hormone (PTH), including its fragments; an antimicrobial; and an anti-fungal agent. Combinations, analogs, fragments, mimetics or polyethylene glycol (PEG)-modified derivatives of these compounds, or other derivatives of any of the above-mentioned substances may also be suitable. Also suitable for use are fusion proteins comprising all or a portion of any of the foregoing proteins. One of ordinary skill in the art, with the benefit of the present disclosure, may recognize additional drugs, including drugs other than proteins or polynucleotides, that may be useful in the compositions and methods of the present disclosure. Such drugs are still considered to be within the spirit of the present disclosure.

Anti-cancer therapeutic agents: In embodiments, one or more anti-cancer therapies can be included in the pharmaceutical compositions and polymeric compositions disclosed herein for administration to an individual. Chemotherapy and anti-cancer agents are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinbiastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are derived from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In aspects, the anti-cancer agent can be selected from remicade, docetaxel, celecoxib, melphalan, dexamethasone (Decadron®), steroids, gemcitabine, cisplatinum, ternozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, docetaxel, pacilitaxel vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil©), paclitaxel, ganciclovir, adriamycin, estrainustine sodium phosphate (Emcyt®), sulindac, or etoposide.

In aspects, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

Small molecule chemical compounds: in aspects, one or more small molecule chemical compounds (for example, antibiotics or vitamins) or can be included in the pharmaceutical compositions and polymeric compositions disclosed herein for administration to an individual. Small molecules are typically organic molecules other than binding polypeptides or antibodies as described above. Small molecules can be identified and chemically synthesized using any one of several well-known methodologies (see, e.g., PCT Application Publication Nos. WO 00/00823 and WO 00/39585, incorporated by reference herein). Small molecules are usually less than about 2000 Daltons in size, such as less than about 1500, 750, 500, 250 or 200 Daltons in size. Small molecules that are capable of binding to a target can be identified using well known techniques (see, e.g., PCT Application Publication Nos. WO 00/00823 and WO 00/39585, incorporated by reference herein).

Small molecules contemplated within the scope of the disclosure include, without limitation, aldehydes, ketones, oximes, hydrazones, semicatbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like. In embodiments, the small molecule(s) contemplated for use with the present invention are components of a combinatorial chemical library. Combinatorial chemical libraries are a collection of multiple species of chemical compounds comprised of smaller subunits or monomers. Combinatorial libraries come in a variety of sizes and can include oligomeric and polymeric libraries comprised of compounds such as carbohydrates, oligonucleotides, and small organic molecules, etc. Such libraries have a variety of uses, such as immobilization and chromatographic separation of chemical compounds, as well as uses for identifying and characterizing ligands capable of binding a target molecule or mediating a biological activity of interest.

Cell-based therapies: In aspects, the pharmaceutical compositions and polymer compositions disclosed herein may comprise or contain one or more therapeutic cells. The cells can be derived from natural sources (for example, blood or bone marrow) or can be engineered to express one or more genes or desired therapeutic proteins.

In some embodiments, the therapeutic cells are autologous or allogenic stem cells. In recent years, high-dose chemotherapy with autologous hematopoietic stem-cell transplantation has become the preferred treatment for certain cancers such as multiple myeloma, non-Hodgkin lymphoma, Hodgkin lymphoma, and leukemia. While not curative, this procedure does prolong overall survival and complete remission. Prior to stem-cell transplantation, patients receive an initial course of induction chemotherapy. The most common induction regimens used today are thalidomide-dexamethasone, bortezomib based regimens, and lenalidomide-dexamethasone (Kyle & Rajkurnar, Blood, 111 (6): 2962-72, 2008). For example, autologous peripheral stem cell transplantation is useful for up to 50% of multiple myeloma patients.

Allogenic transplant (the transplantation of a healthy person's stem cells into the affected individual), is another therapy option for treating cancers. For example, most studies evaluating its use in multiple myeloma patients demonstrate long-term disease-free survival of 10-20%, with a significant fraction of patients developing relapse.

When included as a treatment for suppressing or preventing metastasis according to any of the methods disclosed herein, autologous stem cell transplantation can also include the step of treating the hematopoietic stem-cells and/or bone marrow to be transplanted into the affected individual with any of the anti-cancer agents disclosed herein, prior to transplantation into the affected individual.

Nanoparticles: in aspects, the pharmaceutical compositions and polymers of the present disclosure include a nanoparticle or polymer with a positive charge. A wide variety of nanoparticies or polymers may he used including but not limited to chitosan, poly(ethyleneimine), poly(amidoamine), or a poly(aminoalkyl methacrylate). The nanoparticles or polymers may comprise one or more amino groups which are protonated at the pH of the solution to give the nanoparticle a positive charge. In some particular embodiments, the nanoparticies comprises one more poly(aminoalkyl methacrylate). Some examples of poly(aminoalkyl methacrylate) include poly(2-(diethylaminoethyl)methacrylate) and poly(2-(dimethylaminoethyl)methacrylate). In some embodiments, the methacrylate has been substituted with an amino containing alkyl chain. The amino group can be a primary, secondary (e.g. alkylamine), or tertiary (e.g. dialkylamine) amine. The amino substituted alkyl chain has between 1 and 12 total carbon atoms in some embodiments. In other embodiments, the amino substituted alkyl chain has between 1 and 8 total carbon atoms. In some embodiments, the nanoparticles may have a size from about 50-200 nm.

Methods of Use

Provided herein are methods for delivering a therapeutic agent to an individual in need thereof comprising administering the pharmaceutical compositions and polymer compositions described herein to the individual. In embodiments, the therapeutic agent is delivered to a tissue of an individual in need thereof.

The pharmaceutical compositions and polymer compositions can be administered to the individual via any route known in the art including, without limitation, by injection, inhalation, or insufflation. The liquid forms in which the compositions can be incorporated for administration by injection include aqueous solutions, as well as elixirs and similar pharmaceutical vehicles. Parenteral routes of administration include but are not limited to direct injection into a central venous line, intravenous, intramuscular, intraperitoneal, intraderm al, or subcutaneous injection. The pharmaceutical compositions and polymeric compositions suitable for parenteral administration disclosed herein are generally formulated in USP water or water for injection and may further comprise pH buffers, salts bulking agents, preservatives, and other pharmaceutically acceptable excipients. Nanocarrier complexes, microcarrier complexes, or encapsulates, for parenteral injection may be formulated in pharmaceutically acceptable sterile isotonic solutions such as saline and phosphate buffered saline for injection.

In embodiments, the pharmaceutical compositions and polymeric compositions disclosed herein can be administered directly to a specific tissue. The tissue can be neural tissue, kidney tissue, bladder tissue, skin, heart tissue, lung tissue, lymphatic tissue, pancreatic tissue, splenic tissue, prostatic tissue, breast tissue, testicular tissue, ovarian tissue, uterine tissue, tissue lining a resection cavity wall, olfactory tissue, mucosal tissue, cervical tissue, or cancerous tissue (e.g., a tumor).

Upon coming into contact with the tissue, the gelatinous form of the pharmaceutical compositions and polymeric compositions disclosed herein decreases in viscosity by any of about 5%, 10%, 15%, 20%, 25%, 30%, 35%, 40%, 45%, 50%, 55%, 60%, 65%, 70%, 75%, 80%, 85%, 90%, 95%, or 100%, inclusive of percentages falling in between these values. With respect to therapeutic agents or therapeutic cells embedded in any of the pharmaceutical compositions and polymeric compositions disclosed herein, following decrease in the viscosity of the gel, the therapeutic agents or therapeutic cells embedded in the gel move towards the tissue (such as cancer tissue). In embodiments, the gel can degrade to water-soluble or water insoluble components within about 1-5, 2-4, 1-3, 2-5, 3-5, or 1-2 hours, such as any of about 1, 2, 3, 4, or 5 hours after coming into contact with the tissue. In embodiments, the gel can degrade to water-soluble or water insoluble components within about 1-3 days, such as any of about 12 hours, 24 hours, 36 hours, 48 hours, 60 hours, or 72 hours after coming into contact with the tissue. In embodiments, the gel can degrade to water-soluble or water insoluble components within about 3-7, 4-7, 5-7, 6-7, 3-6, 4-6, 5-6, or any of about 1, 2, 3, 4, 5, 6, or 7 days or more after coming into contact with the tissue. In embodiments, the gel can degrade to water-soluble or water insoluble components within about 30, 45, 60, 75, or 90 minutes of irradiation with light or exposure to an oxidizing agent.

In embodiments, the pharmaceutical compositions and polymeric compositions disclosed herein can be administered to an individual with a cancer, using or in combination with any of the anti-cancer therapeutic agents disclosed herein. In embodiments of the methods herein the cancer may be a solid cancer or a non-solid cancer. In embodiments, the cancer is a solid cancer. Examples of solid cancers contemplated herein include, without limitation, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, brain cancer, cervical cancer, ovarian cancer, liver cancer, sarcoma, bladder cancer, Glioblastoma multiforme, hepatoma, breast cancer, colon cancer, colorectal cancer, endometrial or uterine carcinoma, oralpharyngeal cancer, salivary gland carcinoma, renal cancer, liver cancer, prostate cancer, vulval cancer, thyroid cancer, hepatic carcinoma, gastric cancer, melanoma, and various types of head and neck cancer.

With respect to treatment of cancer, the methods disclosed herein can be practiced in an adjuvant setting. “Adjuvant setting” can refers to a clinical setting in which an individual has had a hi story of cancer, and generally (but not necessarily) been responsive to therapy, which includes, but is not limited to, surgery (such as surgical resection), radiotherapy, and chemotherapy. However, because of their history of the cancer (such as bladder cancer or Glioblastoma multiforme), these individuals are considered at risk of development of cancer. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease (cancer) when first treated.

The methods provided herein may also be practiced in a “neoadjuvant setting,” that is, the method may be carried out before the primary/definitive therapy. In aspects, the subject has previously been treated. In embodiments, the subject has not previously been treated. In embodiments, the treatment is a first line therapy. In embodiments, provided herein is a method for treating or effecting prophylaxis of cancer comprising administering to a subject having or at risk of cancer a therapeutically effective amount of any of the polymeric compositions disclosed herein in a neoadjuvant setting.

In aspects, the disclosure provides methods for using the pharmaceutical compositions and the polymeric compositions disclosed herein for inhibiting the symptoms or conditions (disabilities, impairments) associated with cancer (e.g., metastatic cancer or relapsed cancer) as described in detail below. As such, it is not required that all effects of the condition be entirely prevented or reversed, although the effects of the presently disclosed methods likely extend to a significant therapeutic benefit for the individual. As such, a therapeutic benefit is not necessarily a complete prevention or cure for the condition, but rather, can encompass a result which includes reducing or preventing the symptoms that result from cancer (e.g., metastatic cancer or relapsed cancer), reducing or preventing the occurrence of such symptoms (either quantitatively or qualitatively), reducing the severity of such symptoms or physiological effects thereof, and/or enhancing the recovery of the individual after experiencing cancer (e.g., metastatic cancer or relapsed cancer) symptoms.

Specifically, the therapies (e.g., administration of the pharmaceutical compositions and polymeric compositions disclosed herein), when administered to an individual, can treat or prevent one or more of the symptoms or conditions associated with cancer and/or reduce or alleviate symptoms of or conditions associated with this disorder. As such, protecting an individual from the effects or symptoms resulting from cancer includes both preventing or reducing the occurrence and/or severity of the effects of the disorder and treating a patient in which the effects of the disorder are already occurring or beginning to occur. A beneficial effect can easily be assessed by one of ordinary skill in the art and/or by a trained clinician who is treating the patient. Preferably, there is a positive or beneficial difference in the severity or occurrence of at least one clinical or biological score, value, or measure used to evaluate such individual in those who have been treated with the methods of the present invention as compared to those that have not.

In aspects, the pharmaceutical compositions and polymeric compositions disclosed herein are administered in conjunction with one or more additional anticancer therapies, such as radiation therapy or surgery. As used herein, the term “radiation therapy” refers to the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy partides (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient), External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation. Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may comprise fractionated individual doses of 2 Gy. Dosage ranges for radioisotopes vary widely, and depends on the half-life of the isotope and the strength and type of radiation emitted. When the radiation comprises use of radioactive isotopes, the isotope may be conjugated to a targeting agent, such as a therapeutic antibody, which carries the radionucleotide to the target tissue (e.g., tumor tissue).

Surgery, as described herein, includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and micropically controlled surgery (Mohs surgery). Removal of precancers or normal tissues is also contemplated herein.

Kits, Syringes, and Catheters

Also provided herein are kits including the pharmaceutical compositions and polymeric compositions disclosed herein and written instructions for storing and/or administering the pharmaceutical compositions and polymeric compositions to an individual. The kit can also include one or both of a syringe or catheter as well as one or more therapeutic agents, such as any of the therapeutic agents disclosed herein (including anti-cancer agents). The therapeutic (which may be embedded in the polymeric composition) can be one or more of an antibody or functional fragment thereof, a small molecule chemical compound, an inhibitory nucleic acid, a polypeptide, a nanoparticle, or a cell (such as a stem cell or an engineered cell (e.g., a cell engineered to produce a specific enzyme)). Optionally in conjunction with the syringe, the kit can include a needle, such as an 18-23 gauge needle, such as any of an 18, 19, 20, 21, 22, or 23 gauge needle. In embodiments, the kit includes instructions to store the pharmaceutical composition or polymeric composition at a temperature from about 0° C. to about 35° C. prior to use. In embodiments, the kit is stored at a temperature from about 0° C. to about 35° C. prior to use. In embodiments, the kit is stored at a temperature from about 0° C. to about 35° C. until immediately prior to use.

In aspects, also provided herein is a syringe including the pharmaceutical compositions and polymeric compositions disclosed herein. The syringe can also include one or more therapeutic agents herein (including anti-cancer agents). The therapeutic may further be embedded in the polymeric composition and can be one or more of an antibody or functional fragment thereof, a small molecule chemical compound, an inhibitory nucleic acid, a polypeptide, a nanoparticle, or a cell (such as a stem cell or an engineered cell (e.g., a cell engineered to produce a specific enzyme)). In embodiments, the syringe is stored at a temperature from about 0° C. to about 35° C. prior to use. In embodiments, the syringe is stored at a temperature from about 0° C. to about 35° C. until immediately prior to use. The syringe can have a needle. The needle can be an 18-23 gauge needle, such as any of an 18, 19, 20, 21, 22, or 23 gauge needle.

In aspects, provided herein are catheters including the pharmaceutical compositions or polymeric compositions disclosed herein. The catheter can also include one or more therapeutic agents herein (including anti-cancer agents). The therapeutic may further be embedded in the polymeric composition and can be one or more of an antibody or functional fragment thereof, a small molecule chemical compound, an inhibitory nucleic acid, a polypeptide, a nanoparticle, or a cell (such as a stem cell or an engineered cell (e.g., a cell engineered to produce a specific enzyme)). In embodiments, the catheter is stored at a temperature from about 0° C. to about 35° C. prior to use. In embodiments, the catheter is stored at a temperature from about 0° C. to about 35° C. until immediately prior to use.

### Embodiments 1-74

Embodiment 1. A polymer composition comprising a plurality of non-cross linked polymer blocks, wherein the polymer composition forms a semi-solid gel at physiological temperatures and is water-soluble at temperatures between about 0° C. to about 35° C.; wherein each of the non-cross linked polymer blocks comprise a labile bond separating one or more polymer subunits.

Embodiment 2. The polymer composition of Embodiment 1, wherein the polymer composition forms a semi-solid gel at 37° C.

Embodiment 3. The polymer composition of Embodiment 2, wherein the gel degrades upon cleavage of the labile bond.

Embodiment 4 The polymer composition of any one of Embodiments 1-3, wherein the polymer composition is water-soluble at 25° C.

Embodiment 5. The polymer composition of any one of Embodiments 1-4, wherein the polymer composition is injectable at 25° C.

Embodiment 6. The polymer composition of any one of Embodiments 1-5, wherein the polymer is a copolymer.

Embodiment 7. The polymer composition of Embodiment 6, wherein the polymer is a block copolymer of hydrophilic and relatively hydrophobic polymer blocks.

Embodiment 8. The polymer composition of Embodiment 6 or Embodiment 7, wherein (a) the hydrophilic blocks comprise one or more of ethylene glycol, 1,2-propylene glycol, 1,3-propylene glycol, 1,4-butanediol or any combination thereof; and (b) the relatively hydrophobic blocks comprise one or more of lactic acid, glycolic acid, hydroxybutyric acid, hydroxyvaleric acid, hydroxycaproic acid or any combination thereof.

Embodiment 9. The polymer composition of Embodiment 8, wherein the copolymer is selected from the group consisting of a polylactic acid (PLA)-polyethylene glycol (PEG) di-block copolymer, a PLA-PEG-PLA tri-block copolymer, a PEG-PLA-PEG triblock copolymer, and a PEG-PLA multi-block copolymer.

Embodiment 10. The polymer composition of Embodiment 8, wherein the copolymer is selected from the group consisting of a polylactic acid-co-glycolic acid (PLGA)-polyethylene glycol (PEG) di-block copolymer, a PLGA-PEG-PLGA tri-block copolymer, a PEG-PLGA-PEG triblock copolymer, and a PEG-PLGA multi-block copolymer.

Embodiment 11. The polymer composition of Embodiment 8, wherein the copolymer is selected from the group consisting of a poly(1,2-propylene glycol) (PPG)-polyethylene glycol (PEG) di-block copolymer, a PPG-PEG-PPG tri-block copolymer, a PEG-PPG-PEG triblock copolymer, and a PEG-PPG multi-block copolymers.

Embodiment 12. The polymer composition of Embodiment 8, wherein the copolymer is selected from the group consisting of a polycaprolactone (PCL)-polyethylene glycol (PEG) di-block copolymer, a PCL-PEG-PCL tri-block copolymer, a PEG-PCL-PEG triblock copolymer, and a PEG-PCL multi-block copolymers.

Embodiment 13. The polymer composition of any one of Embodiments 1-12, wherein the labile bond is susceptible to cleavage by one or more of hydrolysis, an enzyme, photosensitivity, a specific pH, and/or soundwaves.

Embodiment 14. The polymer composition of Embodiment 8, wherein the labile bond is susceptible to cleavage by hydrolysis.

Embodiment 15. The polymer composition of Embodiment 14, wherein the labile bond is susceptible to cleavage by rapid hydrolysis compared to other hydrolysable bonds in the block polymers.

Embodiment 16. The polymer composition of Embodiment 14 or Embodiment 15, wherein the bond is an anhydride bond, a glycolic-glycolic ester bond, an imine bond, or a carboxyanhydride bond.

Embodiment 17. The polymer composition of Embodiment 13, wherein the labile bond is susceptible to cleavage by an enzyme.

Embodiment 18. The polymer composition of Embodiment 17, wherein the enzyme is a protease.

Embodiment 19.The polymer composition of Embodiment 18, wherein the protease is a caspase, an esterase, a cysteine-protease, or a cathepsin.

Embodiment 20. The polymer composition of any one of Embodiments 17-19, wherein the bond is a peptide bond.

Embodiment 21. The polymer composition of any one of Embodiments 11-13, wherein the enzyme is released by a cell.

Embodiment 22. The polymer composition of Embodiment 21, wherein the cell is a neural cell, a bladder cell, a stem cell, a therapeutic cell, an engineered cell, a cancer cell, or an immune cell.

Embodiment 23. The polymer composition of Embodiment 22, wherein the cell is a cancer cell.

Embodimeni 24. The polymer composition of Embodiment 13, wherein the labile bond is susceptible to cleavage by changes in pH.

Embodiment 25. The polymer composition of Embodiment 24, wherein the pH is the pH inside infected tissue.

Embodiment 26. The polymer composition of Embodiment 25, wherein the infected tissue is a tumor.

Embodiment 27. The polymer composition of Embodiment 13, wherein the labile bond is susceptible to cleavage by photosensitivity.

Embodiment 28. The polymer composition of Embodiment 27, wherein the bond comprises an azobenzene, a triphenylmethane leucohydroxide, a nitrobenzyl, or a cinnamate.

Embodiment 29. The polymer composition of Embodiment 6 or Embodiment 13, wherein the polymer is the polymer of formula I:

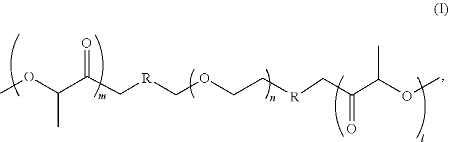

wherein R is one or more of

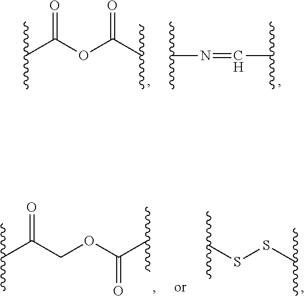

and wherein m, n, and l are one or more polymeric subunits.

Embodiment 30. The polymer composition of Embodiment 6 or Embodiment 13, wherein the polymer is the polymer of formula II:

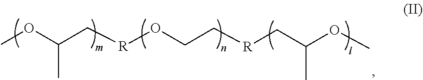

wherein R is one or more of

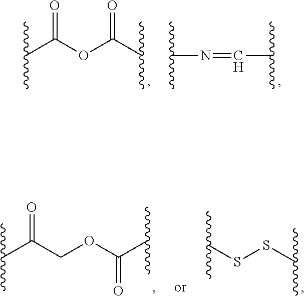

and wherein m, n, and l are one or more polymeric subunits.

Embodiment 31. The polymer composition of Embodiment 6 or Embodiment 13, wherein the polymer is the polymer of formula III:

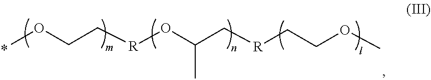

wherein R is one or more of

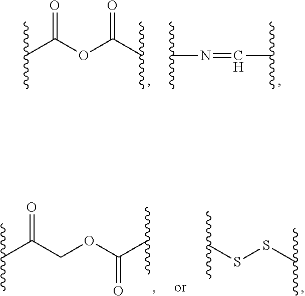

and wherein m, n, and l are one or more polymeric subunits.

Embodiment 32. The polymer composition of Embodiment 6 or Embodiment 13, wherein the polymer is the polymer of formula IV:

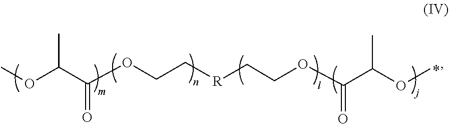

wherein R is one or more of

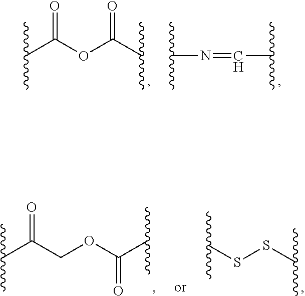

and wherein m, n, l, and j are one or more polymeric subunits.

Embodiment 33. The polymer composition of Embodiment 6 or Embodiment 13, wherein the polymer is the polymer of formula V:

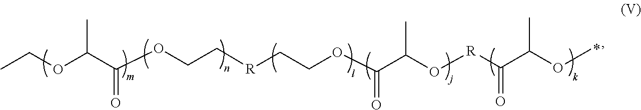

wherein R is one or more of

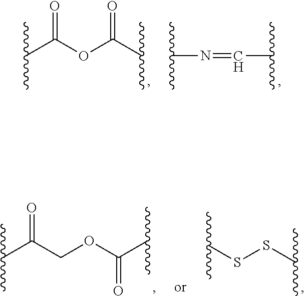

and wherein m, n, l, j, and k are one or more polymeric subunits.

Embodiment 34. The polymer composition of any one of Embodiments 1-33, wherein bond cleavage at physiological temperatures results in degradation of the gel into a 1:2 mixture of water-soluble to water insoluble components.

Embodiment 35. The polymer composition of any one of Embodiments 1-28 or 34, wherein bond cleavage at physiological temperatures results in degradation of the gel into only water-soluble components.

Embodiment 36. The polymer composition of any one of Embodiments 1-35, further comprising a therapeutic embedded in the gel.

Embodiment 37. The polymer composition of Embodiment 36, wherein the therapeutic is one or more of an antibody or functional fragment thereof, a small molecule chemical compound, an inhibitory nucleic acid, a polypeptide, a nanoparticle, a contrasting agent, or a cell.

Embodiment 38. The polymer composition of Embodiment 37, wherein the cell is a stem cell or an engineered cell.

Embodiment 39. The polymer composition of Embodiment 38, wherein nanoparticles are bound to the cell surface and/or included in the cell.

Embodiment 40. The polymer composition of any one of Embodiments 37-39, wherein the nanoparticles are biodegradable.

Embodiment 41. The polymer composition of any one of Embodiments 37-40, wherein the nanoparticles contain an active agent or a contrast agent.

Embodiment 42. The polymer composition of Embodiment 41, wherein the nanoparticles release the active agent for a period of at least about 2 days.

Embodiment 43. A method for delivering a therapeutic to a tissue in an individual in need thereof comprising administering the therapeutic embedded in the polymer composition of any one of Embodiments 1-42 to the individual.

Embodiment 44. The method of Embodiment 43, wherein the polymer is administered via injection.

Embodiment 45. The method of Embodiment 43 or Embodiment 44, wherein the therapeutic is administered directly to the tissue.

Embodiment 46. The method of any one of Embodiments 43-45, wherein the therapeutic is one or more of an antibody or functional fragment thereof, a small molecule chemical compound, an inhibitory nucleic acid, or a cell.

Embodiment 47. The method of Embodiment 46, wherein the cell is a stem cell or an engineered cell.

Embodiment 48. The method of any one of Embodiments 43-47, wherein the tissue is neural tissue, bladder tissue, tumor tissue, tissue lining a resection cavity wall, olfactory tissue, or mucosal tissue.

Embodiment 49. The method of any one of Embodiment 43-48 wherein viscosity of the gel decreases immediately upon coming in contact with the tissue.

Embodiment 50. The method of Embodiment 49, wherein cells embedded in the gel move towards the tissue after the viscosity of the gel decreases.

Embodiment 51. The method of any one of Embodiments 43-50, wherein the gel degrades to water-soluble or water insoluble components within about 1-5 hours after of coming into contact with the tissue.

Embodiment 52. The method of any one of Embodiments 43-50, wherein the gel degrades into a precipitate or non-viscous aqueous solution within 1-3 days of coming into contact with the tissue.

Embodiment 53. The method of any one of Embodiments 43-50, wherein the gel degrades to a water-soluble or water insoluble components within one week of coming into contact with the tissue.

Embodiment 54. The method of any one of Embodiments 43-50, wherein the gel degrades to water-soluble or water insoluble components within one hour of irradiation with light or exposure to an oxidizing agent.

Embodiment 55. A kit comprising: a) the polymer composition of any one of Embodiments 1-42; and b) written instructions for administering the polymer to an individual.

Embodiment 56. The kit of Embodiment 55, further comprising c) a syringe and/or d) a catheter.

Embodiment 57. The kit of Embodiment 55 or Embodiment 56, further comprising e) one or more therapeutic agents.

Embodiment 58. The kit of Embodiment 57, wherein the one or more therapeutic agents are embedded in the polymer.

Embodiment 59. The kit of Embodiment 57 or Embodiment 58, wherein the therapeutic is one or more of an antibody or functional fragment thereof, a small molecule chemical compound, an inhibitory nucleic acid, a polypeptide, a nanoparticle, or a cell.

Embodiment 60. The kit of Embodiment 59, wherein the cell is a stem cell or an engineered cell.

Embodiment 61. The kit of any one of Embodiment 55-60, wherein the polymer is stored at a temperature of between 0-35° C. until immediately prior to use.

Embodiment 62. A syringe comprising the polymer composition of any one of Embodiments 1-42.

Embodiment 63. The syringe of Embodiment 62, further comprising one or more therapeutic agents.

Embodiment 64. The syringe of Embodiment 63, wherein the one or more therapeutic agents are embedded in the polymer.

Embodiment 65. The syringe of Embodiment 63 or Embodiment 64, wherein the therapeutic is one or more of an antibody or functional fragment thereof, a small molecule chemical compound, a polypeptide, an inhibitory nucleic acid, a nanoparticle, or a cell.

Embodiment 66. The syringe of Embodiment 65, wherein the cell is a stem cell or an engineered cell.

Embodiment 67. The syringe of any one of Embodiment 62-66, wherein the syringe is stored at a temperature of between 0-35° C. until immediately prior to use.

Embodiment 68. The syringe of any one of Embodiments 62-67, further comprising an 18-23 gauge needle.

Embodiment 69. A catheter comprising the polymer composition of any one of Embodiments 1-42.

Embodiment 70. The catheter of Embodiment 69, further comprising one or more therapeutic agents.

Embodiment 71. The catheter of Embodiment 70, wherein the one or more therapeutic agents are embedded in the polymer.

Embodiment 72. The catheter of Embodiment 70 or Embodiment 71, wherein the therapeutic is one or more of an antibody or functional fragment thereof, a small molecule chemical compound, an inhibitory nucleic acid, a nanoparticle, a polypeptide, or a cell.

Embodiment 73. The catheter of Embodiment 72, wherein the cell is a stem cell or an engineered cell.

Embodiment 74. The catheter of any one of Embodiment 69-73, wherein the catheter is stored at a temperature of between 0-35° C. until immediately prior to use.

Embodiments N1-N77

Embodiment N1. A polymer composition comprising a non-crosslinked polymer, wherein the non-crosslinked polymer comprises a labile bond linking one or more polymer subunits.

Embodiment N2. The polymer composition of Embodiment 1, comprising a plurality of non-crosslinked polymers.

Embodiment N3. The polymer composition of Embodiment 1 or 2, wherein the polymer composition is a semi-solid gel at a physiological temperature.

Embodiment N4. The polymer composition of any one of Embodiments 1 to 3, wherein the polymer composition is water-soluble at a temperature from about 0° C. to about 35° C.

Embodiment N5. The polymer composition of any one of Embodiments 1 to 4, wherein the polymer degrades upon cleavage of the labile bond.

Embodiment N6. The polymer composition of any one of Embodiments 1 to 5, wherein the polymer composition is injectable at room temperature.

Embodiment N7. The polymer composition of any one of Embodiments 1 to 6, wherein the polymer is a copolymer.

Embodiment N8. The polymer composition of Embodiment 7, wherein the copolymer comprises a hydrophilic polymer subunit and a relatively hydrophobic polymer unit.

Embodiment N9. The polymer composition of Embodiment 7, wherein the copolymer comprises: (a) ethylene glycol, 1,2-propylene glycol, 1,3-propylene glycol, 1,4-butanediol, or a combination of two or more thereof; and (b) lactic acid, glycolic acid, caprolactone, hydroxybutyric acid, hydroxyvaleric acid, hydroxycaproic acid, or a combination of two or more thereof.

Embodiment N10.The polymer composition of Embodiment 7, wherein the copolymer comprises: (a) ethylene glycol; and (b) 1,2-propylene glycol, 1,3-propylene glycol, 1,4-butanediol, lactic acid, glycolic acid, caprolactone, hydroxybutyric acid, hydroxyvaleric acid, hydroxycaproic acid, or a combination of two or more thereof.

Embodiment N11. The polymer composition of Embodiment 7, wherein the copolymer comprises: (a) ethylene glycol; and (b) 1,2-propylene glycol, 1,3-propylene glycol, lactic acid, glycolic acid, caprolactone, or a combination of two or more thereof.

Embodiment N12. The polymer composition of Embodiment 7, wherein the copolymer comprises: (a) ethylene glycol; and (b) 1,2-propylene glycol, 1,3-propylene glycol, or a combination thereof.

Embodiment N13. The polymer composition of Embodiment 7, wherein the copolymer comprises: (a) ethylene glycol; and (b) 1,2-propylene glycol.

Embodiment N14. The polymer composition of Embodiment 7, wherein the copolymer comprises: (a) ethylene glycol; and (b) 1,3-propylene glycol.

Embodiment N15. The polymer composition of Embodiment 7, wherein the copolymer comprises: (a) ethylene glycol; and (b) lactic acid, glycolic acid, caprolactone, or a combination of two or more thereof.

Embodiment N16. The polymer composition of Embodiment 7, wherein the copolymer comprises: (a) ethylene glycol; and (b) lactic acid.

Embodiment N17. The polymer composition of Embodiment 7, wherein the copolymer comprises: (a) ethylene glycol; and (b) glycolic acid.

Embodiment N18. The polymer composition of Embodiment 7, wherein the copolymer comprises: (a) ethylene glycol; and (b) lactic acid and glycolic acid.

Embodiment N19. The polymer composition of Embodiment 7, wherein the copolymer comprises: (a) ethylene glycol; and (b) caprolactone.

Embodiment N20. The polymer composition of Embodiment 7, wherein the copolymer is a polylactic acid-polyethylene glycol di-block copolymer, a polylactic acid-polyethylene glycol-polylactic acid tri-block copolymer, a polyethylene glycol-polylactic acid-polyethylene glycol triblock copolymer, or a polyethylene glycol-polylactic acid multi-block copolymer.

Embodiment N21. The polymer composition of Embodiment 7, wherein the copolymer is a polyglycolic acid-polyethylene glycol di-block copolymer, a polyglycolic acid-polyethylene glycol-polyglycolic acid tri-block copolymer, a polyethylene glycol-polyglycolic acid-polyethylene glycol triblock copolymer, or a polyethylene glycol-polyglycolic acid multi-block copolymer.

Embodiment N22. The polymer composition of Embodiment 7, wherein the copolymer is a poly(lactic-co-glycolic acid)-polyethylene glycol di-block copolymer, a poly(lactic-co-glycolic acid)-polyethylene glycol-poly(lactic-co-glycolic acid) tri-block copolymer, or a polyethylene glycol-poly(lactic-co-glycolic acid) multi-block copolymer.

Embodiment N23. The polymer composition of Embodiment 7, wherein the copolymer is a poly(1,2-propylene glycol)-polyethylene glycol di-block copolymer, a poly(1,2-propylene glycol)-polyethylene glycol-poly(1,2-propylene glycol) tri-block copolymer, a polyethylene glycol-poly(1,2-propylene glycol)-polyethylene glycol triblock copolymer, or a polyethylene glycol-poly(1,2-propylene glycol) multi-block copolymer.

Embodiment N24. The polymer composition of Embodiment 7, wherein the copolymer is a poly(1,3-propylene glycol)-polyethylene glycol di-block copolymer, a poly(1,3-propylene glycol)-polyethylene glycol-poly(1,3-propylene glycol) tri-block copolymer, a polyethylene glycol-poly(1,3-propylene glycol)-polyethylene glycol triblock copolymer, or a polyethylene glycol-poly(1,3-propylene glycol) multi-block copolymer.

Embodiment N25. The polymer composition of Embodiment 7, wherein the copolymer is a polycaprolactone-polyethylene glycol di-block copolymer, a polycaprolactone-polyethylene glycol-polycaprolactone tri-block copolymer, a polyethylene glycol-polycaprolactone-polyethylene glycol triblock copolymer, or a polyethylene glycol-polycaprolactone multi-block copolymer.

Embodiment N26. The polymer composition of any one of Embodiments 1 to 25, wherein the labile bond is cleavable by hydrolysis, an enzyme, photosensitization, a pH change, a soundwave, or a combination of two or more thereof.

Embodiment N27. The polymer composition of Embodiment 26, wherein the labile bond is cleavable by hydrolysis.

Embodiment N28. The polymer composition of Embodiment 26 or 27, wherein the labile bond is an anhydride bond, a glycolic-glycolic ester bond, an imine bond, or a carboxyanhydride bond.

Embodiment N29. The polymer composition of Embodiment 26, wherein the labile bond is cleavable by an enzyme.

Embodiment N30. The polymer composition of Embodiment 29, wherein the enzyme is a protease.

Embodiment N31. The polymer composition of Embodiment 30, wherein the protease is a caspase, an esterase, a cysteine-protease, or a cathepsin.

Embodiment N32. The polymer composition of any one of Embodiments 29 to 31, wherein the labile bond is a peptide bond.

Embodiment N33. The polymer composition of any one of Embodiments 29 to 32, wherein the enzyme is released by a cell.

Embodiment N34. The polymer composition of Embodiment 33, wherein the cell is a neural cell, a bladder cell, a stem cell, a therapeutic cell, an engineered cell, a cancer cell, or an immune cell.

Embodiment N35. The polymer composition of Embodiment 33, wherein the cell is a cancer cell.

Embodiment N36. The polymer composition of Embodiment 26, wherein the labile bond is cleavable by a pH change.

Embodiment N37. The polymer composition of Embodiment 36, wherein the pH change is a pH change inside an infected tissue.

Embodiment N38. The polymer composition of Embodiment 37, wherein the infected tissue is a tumor.

Embodiment N39. The polymer composition of Embodiment 26, wherein the labile bond is cleavable by photosensitization.

Embodiment N40. The polymer composition of Embodiment 39, wherein the labile bond comprises an azobenzene, a triphenylmethane leucohydroxide, a nitrobenzyl, or a cinnamate.

Embodiment N41. The polymer composition of Embodiment 7, wherein the copolymer is of Formula (I):

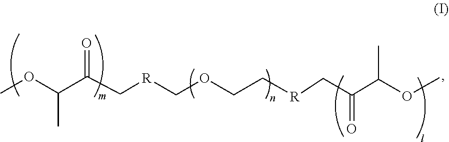

wherein m, n, and l are one or more polymeric subunits; and R is a labile bond.

Embodiment N42. The polymer of Embodiment 41, wherein R is a labile bond cleavable by hydrolysis, an enzyme, photosensitization, a pH change, a soundwave, or a combination of two or more thereof.

Embodiment N43. The polymer of Embodiment 41, wherein R is independently —C(═O)—O—C(═O)—, —N═CH—, —C(═O)—CH₂—O—C(═O)—, or —S—S—.

Embodiment N44. The polymer composition of Embodiment 7, wherein the copolymer is of Formula (II):

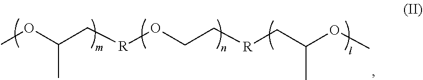

wherein m, n, and l are one or more polymeric subunits; and R is a labile bond.

Embodiment N45. The polymer of Embodiment 44, wherein R is a labile bond cleavable by hydrolysis, an enzyme, photosensitization, a pH change, a soundwave, or a combination of two or more thereof.

Embodiment N46. The polymer of Embodiment 44, wherein R is independently —C(═O)—O—C(═O)—, —N═CH—, —C(═O)—CH₂—O—C(═O)—, or —S—S—.

Embodiment N47. The polymer composition of Embodiment 7, wherein the copolymer is of Formula (III):

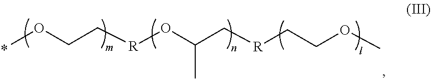

wherein m, n, and l are one or more polymeric subunits; and R is a labile bond.

Embodiment N48. The polymer of Embodiment 47, wherein R is a labile bond cleavable by hydrolysis, an enzyme, photosensitization, a pH change, a soundwave, or a combination of two or more thereof.

Embodiment N49. The polymer of Embodiment 47, wherein R is independently —C(═O)—O—C(═O)—, —N═CH—, —C(═O)—CH₂—O—C(═O)—, or —S—S—.

Embodiment N50. The polymer composition of Embodiment 7, wherein the copolymer is of Formula (IV):

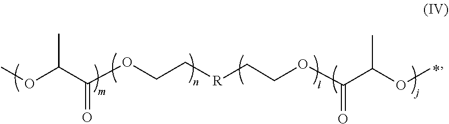

wherein m, n, l, and j are one or more polymeric subunits; R is a labile bond; and * is a terminal group.

Embodiment N51. The polymer of Embodiment 50, wherein R is a labile bond cleavable by hydrolysis, an enzyme, photosensitization, a pH change, a soundwave, or a combination of two or more thereof.

Embodiment N52. The polymer of Embodiment 50, wherein R is independently —C(═O)—O—C(═O)—, —N═CH—, —C(═O)—CH₂—O—C(═O)—, or —S—S—.

Embodiment N53. The polymer composition of Embodiment 7, wherein the copolymer is of Formula (V):

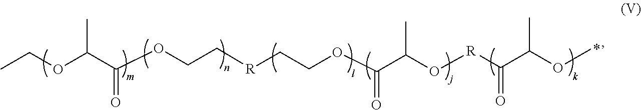

wherein m, n, l, j, and k are one or more polymeric subunits; R is a labile bond; and * is a terminal group.

Embodiment N54. The polymer of Embodiment 53, wherein R is a labile bond cleavable by hydrolysis, an enzyme, photosensitization, a pH change, a soundwave, or a combination of two or more thereof.

Embodiment N55. The polymer of Embodiment 53, wherein R is independently —C(═O)—O—C(═O)—, —N═CH—, —C(═O)—CH₂—O—C(═O)-, or —S—S—.

Embodiment N56. The polymer composition of any one of Embodiments 1 to 55, wherein bond cleavage at a physiological temperature results in degradation of the gel into a 1:2 mixture of water-soluble components to water insoluble components.

Embodiment N57. The polymer composition of any one of Embodiments 1 to 55, wherein bond cleavage at a physiological temperature results in degradation of the gel into only water-soluble components.

Embodiment N58. A pharmaceutical composition comprising the polymer composition of any one of Embodiments 1 to 57 and a therapeutic agent.

Embodiment N59. The pharmaceutical composition of Embodiment 58, wherein the therapeutic agent is an antibody, a functional fragment of an antibody, a small molecule chemical compound, a nucleic acid, a polypeptide, a contrasting agent, a cell, or a combination of two or more thereof.

Embodiment N60. The pharmaceutical composition of Embodiment 58, wherein the therapeutic agent is a stem cell or an engineered cell.

Embodiment N61. The pharmaceutical composition of any one Embodiments 58 to 60, further comprising a pharmaceutically acceptable excipient.

Embodiment N62. A method for delivering a therapeutic agent to a subject in need thereof, the method comprising administering to the subject the pharmaceutical composition of any one of Embodiments 58 to 61.

Embodiment N63. The method of Embodiment 62, wherein the pharmaceutical composition is administered by injection.

Embodiment N64. The method of Embodiment 62 or 63, wherein the pharmaceutical composition is administered to a tissue in the subject.

Embodiment N65. The method of Embodiment 64, wherein the tissue is neural tissue, bladder tissue, tumor tissue, tissue lining a resection cavity wall, olfactory tissue, or mucosal tissue.

Embodiment N66. A kit comprising the polymer composition of any one of Embodiments 1 to 57 and written instructions for administering the polymer composition to a subject.

Embodiment N67. A kit comprising the pharmaceutical composition of any one of Embodiments 58 to 61 and written instructions for administering the pharmaceutical composition to a subject.

Embodiment N68. The kit of Embodiment 66 or 67, further comprising a syringe, a needle, a catheter, or a combination of two or more thereof.

Embodiment N69. The kit of any one of Embodiment 66 to 68, wherein the instructions describe storing the composition at a temperature from about 0° C. to about 35° C. prior to use.

Embodiment N70. The kit of any one of Embodiment 66 to 68, wherein the instructions describe storing the composition at room temperature prior to use.

Embodiment N71. A syringe comprising the polymer composition of any one of Embodiments 1 to 57.

Embodiment N72. A syringe comprising the pharmaceutical composition of any one of Embodiments 58 to 61.

Embodiment N73. The syringe of Embodiment 71 or 72, further comprising an 18 gauge needle, a 19 gauge needle, a 20 gauge needle, a 21 gauge needle, a 22 gauge needle, or a 23 gauge needle.

Embodiment N74. A catheter comprising the polymer composition of any one of Embodiments 1 to 57.

Embodiment N75. A catheter comprising the pharmaceutical composition of any one of Embodiments 58 to 61.

Embodiment N76. A bio-ink comprising the polymer composition of any one of Embodiments 1 to 57.

Embodiment N77. A bio-ink comprising the pharmaceutical composition of any one of Embodiments 58 to 61.

### EXAMPLES

The disclosure a can be further understood by reference to the following examples, which are provided by way of illustration and are not meant to be limiting.

The practice of the disclosure will employ, unless otherwise indicated, conventional techniques of chemistry, polymer chemistry, molecular biology, microbiology, cell biology, biochemistry, nucleic acid chemistry, which are well known to those skilled in the art. Such techniques are explained fully in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Antibodies: A Laboratory Manual, Second edition, Cold Spring Harbor Laboratory Press, Cold Spring Harbor, N.Y. (Greenfield, ed., 2014), Beaucage et al. eds., Current Protocols in Nucleic Acid Chemistry, John Wiley K. Sons. Inc., New York, 2000, (including supplements through 2014); Gene Transfer and Expression in Mammalian Cells (Makrides, ed., Elsevier Sciences B. V., Amsterdam, 2003); Ravve, Principles of Polymer Chemistry (Springer, 1995), and Robeson, Polymer Blends (Hanser Publications, 2007).

### Example 1

Synthesis and characterization of DLPLGA-COOCO-PEG-COOCO-DLPLA responsive gel with fast degrading anhydride bonds. “DLPLGA” and “DL-PLGA” refer to poly(D,L-lactic-co-glycolic acid). “DLPLA,” “DL-PLA,” and “DL-lactic aicd” refer to poly(D,L-lactic acid).

Short chain DLPLAG with carboxylic acid end groups was prepared by melt condensation of DL-lactic acid and glycolic acid at a 6:1 molar ratio, using a drop of phosphoric acid as catalyst. The mixture was heated to 100° C. for 72 hours, applying low vacuum of ˜100 mm Hg. After 72 hours, the reaction mixture was connected to high vacuum pump for 2 hours. A viscous clear polymer was obtain at >90 yield. The molecular weight (MW) of the polymer was 1200 as determined by GPC, 1H-NMR confirmed the LA:GA ratio, IR confirmed ester bonds at 1720 cm⁻¹.

The resulting polymer was reacted with acetic anhydride (10 gram polymer and 25 ml acetic anhydride) at reflux for 2 hours and the acetic anhydride was evaporated to dryness to yield a viscous material. FTIR confirmed anhydride bonds at 1800 and 1740 cm⁻¹ while GPC analysis showed an increase in molecular weight to Mw=2600.

PEG dicarboxylic acid was prepared by mixing PEG diol, number average molecular weight=1000, 1450 and 2000 (all from Sigma-Aldrich) and succinic anhydride (Sigma) at a 1:2.5 OH:anhydride mole ratio in excess toluene. The mixture was heated for 5 hours with stirring and the toluene was evaporated to dryness. The residue was left at 100° C. for 6 hours to affect esterification. The PEG disuccinate was reacted with acetic anhydride at 1:2.5w/v for 60 min and evaporated to dryness to form a viscous material. Anhydride formation was confirmed by IR, with peaks at 1800 and 1740 cm⁻¹. Alternatively, acetate anhydride end groups were obtained by reacting the polymers in dichloromethane and potassium carbonate as described above.

Sebacic acid anhydride pre-polymer was prepared by reacting 10 grams of sebacic acid (Sigma) with acetic anhydride at a 1:2.5 w/v ratio for 60 min. The acetic anhydride was evaporated to dryness to form a solid white material.

Synthesis of polymers: PEG-disuccinate anhydride and sebacic anhydride pre-polymer or/and DLPLGA anhydride pre-polymer were mixed at different w/w ratio of between 10-60% w/w of PEG anhydride, 1-15% sebacic anhydride and 20-60% DLPLGA anhydride. The mixtures were polymerized at 150° C. under high vacuum for 4 hours to form the anhydride containing polymers. The resulted polymers were purified by dispersing the polymer powder in iced water. After 60 minutes of stirring, the insoluble residue was isolated and the aqueous solution was used for gelling experiment and the rest was dried by lyophilization and stored at 20° C. The pure polymer was analyzed by NMR, IR and GPC.

Gelling evaluation: Polymer solutions at 10%, 20% and 30% in deionized iced water were prepared and evaluated for gel formation. The polymer solutions were placed in a water bath at 5° C. and the temperature was increased by 5° C. each time and the gelling point and precipitation temperatures were visualized and determined.

### Example 2

Synthesis and characterization of DLPLGA-PEG-COOCO-PEG-DLPLA responsive gel with fast degrading anhydride bond.

PEG mono carboxylic acid is prepared by selective oxidation of one side or by reacting PEG diol with one equivalent of succinic or maleic anhydride. These HO-PEG-COOH with PEG MWs of 500, 750 and 1000 were used for the block copolymerization with DL-lactide at a 70:30 w/w ratio. The diblock DLPLA-PEG-COOH was reacted with acetic anhydride to form the anhydride bond or reacted with phosgene or phosgene precursor to form the anhydride bond between the two PEG ends. This conjugation can include the formation of a chain of a polyanhydrides of different chain length in between the PEG chains by adding dicarboxylic acid anhydride pre-polymer, such as sebacic anhydride pre-polymer.

Other cleavable bonds can be formed between the two DLPLA-PEG chains such as: PEG-N═C-PEG, PEG-O—-CH₂—COO-PEG; PEG-O—-CH₂—COO-CH₂-O-PEG, which are hydrolytically cleavable. Enzymatically degradable bonds between two PEG chains are incorporated using methods known in the literature. For example, in PEG-CO—CH₂—O—CO-PEG, this type of bond is cleavable by cysteine-proteases. Incorporation of a short peptide that is cleavable by a specific enzyme that is secreted by a certain cell type can be incorporated within two PEG chains.

### Example 3

O-nitrobenzyl (o-NB)-containing polymers: These polymers are sensitive to UV light and cleave upon irradiation into the corresponding o-nitrosobenzaldehyde and free carboxylic acid, as described in Scheme 1. The concept can be expanded to yield organic bases by employing o-nitrobenzyl carbarmates of amines and diamines, which then result in the release of the respective alkylamines.

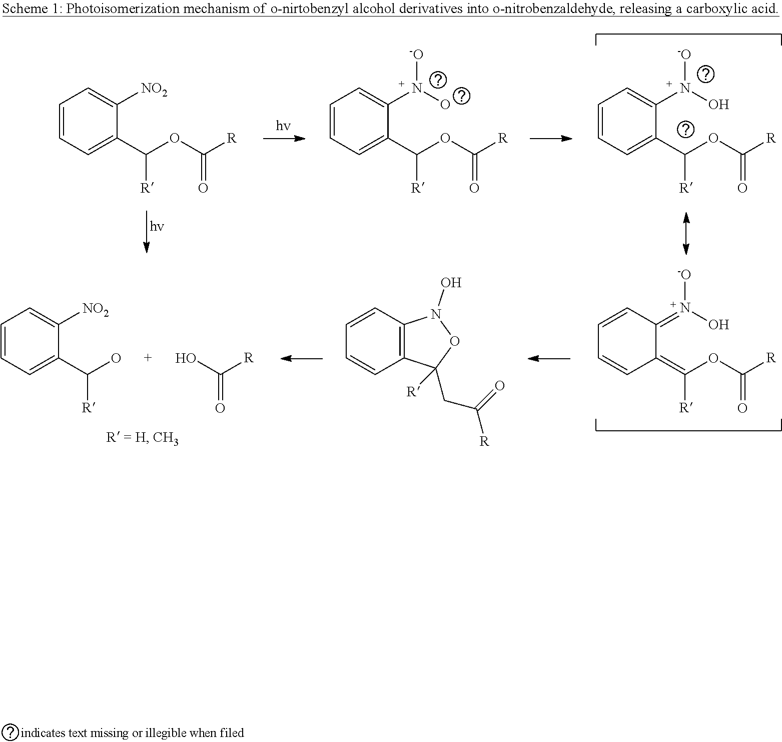

Incorporation of an o-NB group for the development of photo responsive materials is known in the art. Applications using this technology primarily have been focused on the development of micelles and nanoparticles generated out of photo-cleavable amphiphilic block copolymers, for light induced drug delivery, with an o-NB group on the backbone or, alternatively, as an amphiphilic block copolymer with a photo-cleavable o-NB group incorporated along the backbone. In both cases, light triggers disassembly of the micelle or nanoparticle accompanied by burst release of the payload.

Multi-stimuli o-NB-based materials have been reported as well with thermo- and light-sensitive hydrophilic block copolymers, where the micellization process is temperature dependent, due to the polymer thermo-responsive backbone, and the dissociation process induced by UV irradiation, as a result of o-NB cleavage.

Other than for drug delivery, the o-NB group was also exploited for development of biological systems, for example, as a light-triggered cell adhesion and differentiation substrate, protected by a chain PEG linked through o-NB group, which cleave upon irradiation and exposes the substrate for controllable and precise cell differentiation. Finally, hydrogels consisting of PEG with photo-cleavable o-NB junctions have been developed as dynamic cell culture platforms.

In this Example, o-nitrobenzyl functional groups were incorporated into thermoresponsive block copolymers based on PEG and biodegradable polyesters based on lactic, glycolic, hydroxybutyric, caprolactone, and polycarbonates. These thermogel polymers are sensitive to photo cleavage into block components that do not possess gel properties and either precipitate or dissolve in water.

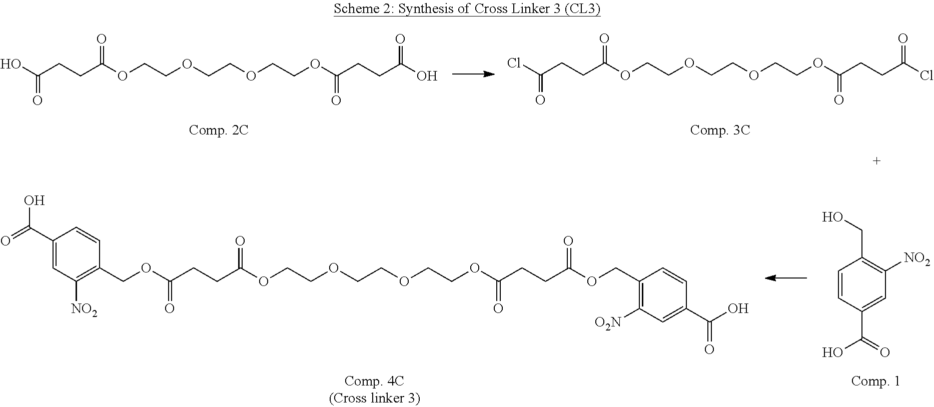 [SMILES: O=C(O)CCC(=O)OCCOCCOCCOC(=O)CCC(=O)O ; O=C(Cl)CCC(=O)OCCOCCOCCOC(=O)CCC(=O)Cl ; O=C(CCC(=O)OCc1ccc(C(=O)O)cc1[N+](=O)[O-])OCCOCCOCCOC(=O)CCC(=O)OCc1ccc(C(=O)O)cc1[N+](=O)[O-] ; O=C(O)c1ccc(CO)c([N+](=O)[O-])c1]

Synthesis of Compound 2C (triethylene glycol-bis-succinic acid ester): Triethylene glycol (5 ml, 0.037 mol) was dissolved in dry DCM (20 ml) followed by addition to succinic anhydride (9 gr, 0.09 mol) and a catalytic amount followed by overnight reflux. For purification, the product was precipitated by adding diethyl ether 2 times then dissolved again in DCM, and washed 3 times with diluted HCl solution, and finally washed with DDW. The product was confirmed by ¹H NMR, and ES-MS.

Synthesis of Compound 3C (triethylene glycol-bis-succinic acyl chloride ester): Triethylene glycol-bis succinic acid ester (400 mg, 1.14 mmol) dissolved in dry DCM (5 ml), added with thionyl chloride (250 μl, 3.42 mmol) stirred overnight at RT under N₂. The product was washed with heptane and confirmed by ¹H NMR.

Synthesis of Compound 4C (Triethylene glycol-bis-succinic NB ester (CL-3)): HM-NBA (200 mg, 1.02 mmol), TEA (300 μl, 2.04 mmol) and DMAP (catalytic amount) were dissolved in dry THF (10 ml). Triethylene glycol-bis-succinic acyl chloride ester (200 mg, 0.51 mmol) was dissolved in dry THF (10 ml), and added dropwise during 2 hours to the reaction mixture, then stirred for 24 Hours under N₂. For purification, DDW was added to the reaction mixture and extracted 3 times with EtOAc.

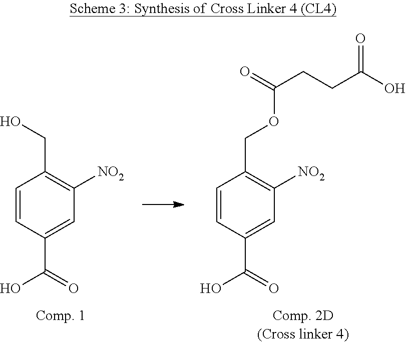 [SMILES: O=C(O)c1ccc(CO)c([N+](=O)[O-])c1 ; O=C(O)CCC(=O)OCc1ccc(C(=O)O)cc1[N+](=O)[O-]]

Synthesis of Compound 2D (CL-4): HM-NBA (500 mg, 2.53 mmol) was dissolved in dry EtOAc (20 ml), added with a TEA (700 μl, 3.81 mmol) and succinic anhydride (253 mg, 2.53 mmol). The reaction mixture was refluxed overnight under dry conditions. For purification, the product was precipitated in cold EtOAc, several times. The product was analyzed by ¹H NMR and ESI-MS.

O-NB Cross Linkers Characterization:

Light induced Change in absorption spectrum of cross linker: Synthetized cross likers were examined for their reaction to UV irradiation. A solution of diluted cross linker in DMSO or methanol (0.03 μg/ml) was prepared. The sample was exposed to UV light using 230V (50/60 Hz), 365 nm UV lamp for different increasing time periods followed by recording of UV—Vis absorption spectra in the range of 200-450 nm on an Ultrospec 2100 pro instrument (Amersham Biosciences).

Electrospray Ionization Mass Spectrometry (ESI-MS): Mass spectrometry is a sensitive analytical technique used to detect, identify and quantitate molecules based on their mass and charge (m/z) ratio, after their conversion to ions. ESI-MS was recorded on a ThermoQuest Finnigan LCQ-Duo instrument

Photo Cleavable PEG-PLA Di-Block Copolymer Synthesis:

Synthesis of Poly(ethylene glycol) methyl ether NB ester: PEG methyl ether 2000 Da (1 gr, 0.5 mmol), DCC (134 mg, 0.65 mmol) and a catalytic amount of DMAP were dissolved in dry previously distilled DCM (20 ml) and cooled to 0° C. HM-NBA 118 mg (0.6 mmol) was dissolved in dry THF (5 ml) and combined with dry DCM (15 ml). This solution was added dropwise during 30 min to the reaction mixture followed by overnight stirring and ending with quenching the reaction with AcOH. For purification, the reaction mixture was cooled and the precipitation filtered. In order to obtain the final product, the solvent was evaporated, which was confirmed by TLC and ¹H NMR.

PLA ROP on o-Me PEG NB ester: Recrystallized L-lactide (450 mg, 3.12 mmol), o-Me PEG NB ester (320 mg, 0.15 mmol) and stannous octoate (catalytic amount) were added to siliconized RBF dissolved in dry EtOAc (10 ml) under dry conditions. Subsequently, the temperature was gradually elevated to 90° C. and the EtOAc was evaporated from the system. Eventually, the temperature was elevated to 160° C. and the reaction mixture was stirred for 6 hr. For purification, the reaction mixture was cooled and dissolved in DCM and precipitated with ethyl ether. The product was analyzed using ¹H NMR.

PLA ROP on isobutanol (Isobutene-PLA synthesis): Recrystallized L-lactide (3 gr, 20.8 mmol), dry distilled isopropanol (45 mg, 0.75 mmol) and stannous octoate (catalytic amount) were reacted by the same procedure described above for ‘PLA ROP on o-Me PEG NB ester’. The product was confirmed by ¹H NMR and GPC.

Isobutene-PLA succinic acid ester synthesis: Isobutene-PLA 3000 Da (1 gr, 0.33 mmol), succinic anhydride (50 mg, 0.5 mmol) and TEA (0.5 mmol) were dissolved in dry DCM and stirred under N₂ for 24 hours. For Purification, the product was crystalized twice with ether. The product was confirmed by ¹H NMR and GPC.

PEG-PLA photo cleavable di-block co polymer synthesis: o-Me PEG NB ester (1 gr, 0.45 mmol), isobutene-PLA-succinic acid ester (1.4 gr, 0.45 mmol), DCC (154 mg, 0.67 mmol) and a catalytic amount of DMAP were dissolved in dry DCM and stirred under dry conditions at 0° C. for 3 hr. For purification, the solution was dialyzed in DCM with 2000 Da cutoff for 2 days. The product was confirmed by ¹H NMR and GPC.

Molecular weight determination: The polymerization of PLA and the coupling with PEG was confirmed by increase in molecular weight. The molecular weights of the polymers were determined by Gel permeation chromatography (GPC). GPC system consisting of a Waters 1515 Isocratic HPLC Pump, with 2410 Refractive Index detector (RI) (Waters, Mass.), a Rheodyne (Coatati, Calif.) injection valve with a 20 μL loop. The samples were eluted with CHCl₃ (HPLC grade) through a Styragel HR4E column (Waters, Mass.) at a flowrate of 1 mL/min. The molecular weights were determined relative to polystyrene standards (Polyscience, Warrington, Pa.) with a molecular-weight range of 500-10,000 Da.

In this Example, o-NB based crosslinkers were synthesized. The aim was to achieve a water-soluble crosslinker that may be used to cross link water-soluble polymers, such as polysaccharides and form a hydrogel. The cross linker molecule consists of carboxylic acids, throughout which the crosslinking will be carried out; at least one o-nitrobenzyl group that will provide the photo-cleavable function; and water solubility of the final molecule, in order to react it in aqueous media with polysaccharides. The last synthesized cross linker molecule (CL-4) met all the requirements, and was used later for the next step of chitosan cross-linking.

Synthesis of Compound 1 (HM-NBA): The synthesis of the starting material, HM-NBA, shared for all synthesized cross linkers, was performed by hydrolysis reaction of commercial BM-NBA according to Scheme 4.

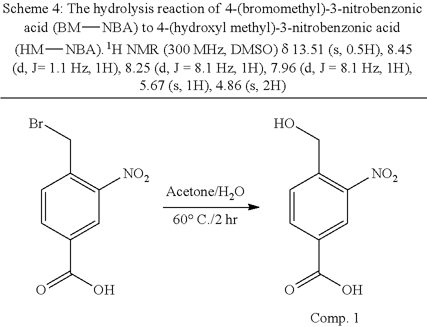

The synthesis of CL-3 was carried out by relying on the synthesis method of CL-2 and replacing the sebacic acid based bridge (that was synthesized as a proof of concept) with a more hydrophilic chain (triethylene glycol). The reaction scheme is described in Scheme 5.

Synthesis of compound 2C (triethylene glycol-bis-succinic acid ester): Starting with triethylene glycol and reacting it from both sides with succinic anhydride, resulting in a dicarboxylic acid (parallel to sebacic acid). ¹H NMR (300 MHz, cdcl₃) δ 10.65 (s, 1H), 4.31-4.19 (m, 2H), 3.69 (s, 2H), 3.63 (d, J=4.1 Hz, 2H), 2.65 (dd, J=5.8, 4.0 Hz, 4H)

Synthesis of compound 3C (triethylene glycol-bis-succinic acyl chloride ester): Triethylene glycol-succinic acid ester chlorination was performed in order to make the molecule available for nucleophilic attack at the next reaction step. 1H NMR (300 MHz, cdcl3) δ 4.26 (dd, J=11.1, 6.3 Hz, 1H), 3.70 (dd, J=4.8, 3.4 Hz, 1H), 3.64 (s, 1H), 3.22 (t, J=6.6 Hz, 1H), 2.70 (dd, J=13.3, 6.8 Hz, 1H)

Synthesis of compound 4C (bis succinic NB Triethylene glycol ester (CL-3)): A nucleophilic attack reaction of HM-NBA (2 equimolar) was performed with triethylene glycol-bis-succinic acyl chloride ester (1 equimolar).

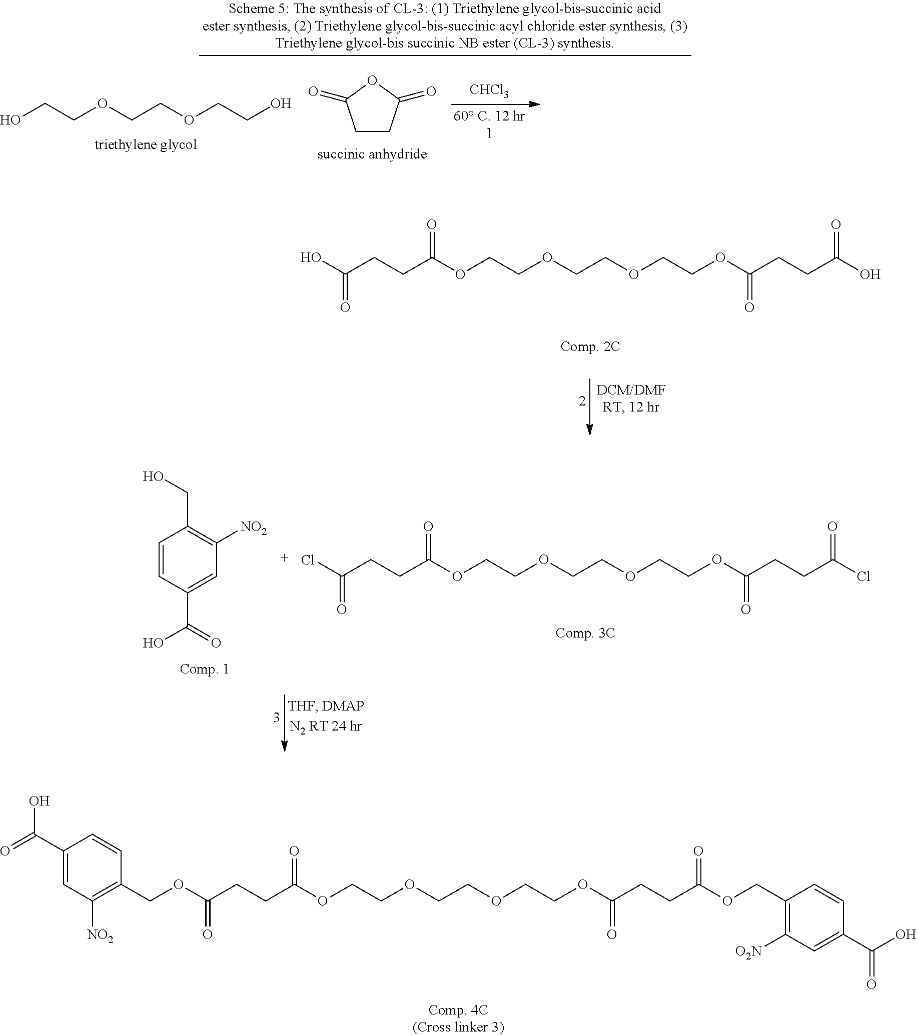

Synthesis of compound 2D (Cross linker 4), NBA succinic acid ester: CL-4 was synthesized by reacting HM-NBA with an equimolar of succinic anhydride, resulting in a molecule with two carboxylic acids and one NB group. This product was water-soluble and therefore met all the requirements for the o-nitrobenzyl based cross linker. Additionally, the method was of high yield, and the product was easily purified. Reaction scheme described in Scheme 6.

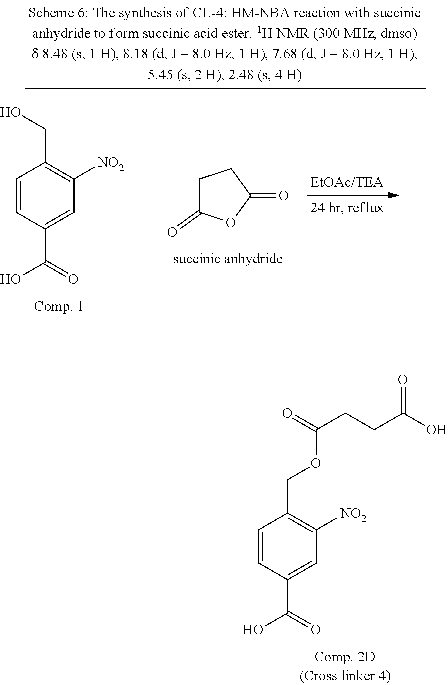

O-NB Based Cross Linkers Characterization:

Effect of UV irradiation on crosslinkers UV absorption spectrum: We examined the effect of UV irradiation and subsequent photo-cleavage properties of the synthetized cross linkers by irradiating the prepared cross linkers using 365 nm UV lamp and monitoring the change in its UV absorbance spectrum.

Irradiation of Cross linker 1 molecule resulted with a change in the absorbance spectrum, with a gradual appearance of two peaks at around 320 nm and 290 nm, as the irradiation period increases. A similar result was obtained for the irradiation of cross linker 2 molecule, where a gradual appearance of a new peak at 250 nm was observed, as the irradiation period increased.

The analysis was performed on the cross linkers final molecule (CL-4) in order to ensure its structure according to its molecular mass. ESI-MS after irradiation was conducted to conform the photo cleavage of the synthetized cross linker corresponds to the reported photo cleavage mechanism of o-NB alcohol derivatives as illustrated in Scheme 7.

Before irradiation (final cross linker molecule), the results show one main peak at 296.90 m/z corresponding to the theoretical molecular mass of the molecule. After irradiation, the results show two main peaks: 234.92 m/z, 116.91 m/z. The first suitable for the reaction of the o-nitrobenzaldehyde after reaction with methanol (the solvent at the analysis) and the second that with a sodium ion with a molecular mass totaling of 234.04 (g/mol).

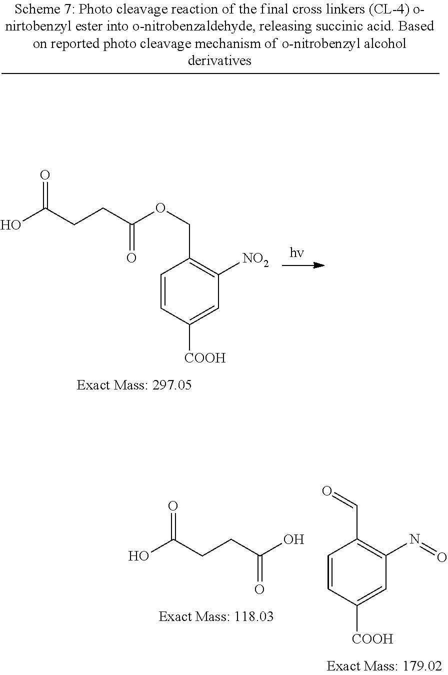

Photo Cleavable PEG-PLA Di-Block Co Polymer Synthesis:

Synthesis of Poly (ethylene glycol) methyl ether NB ester: HM-NBA coupling reaction with Poly (ethylene glycol) was performed with methyl ether, to form an ester bond. This serves as the first block of the photo-responsive block copolymer.

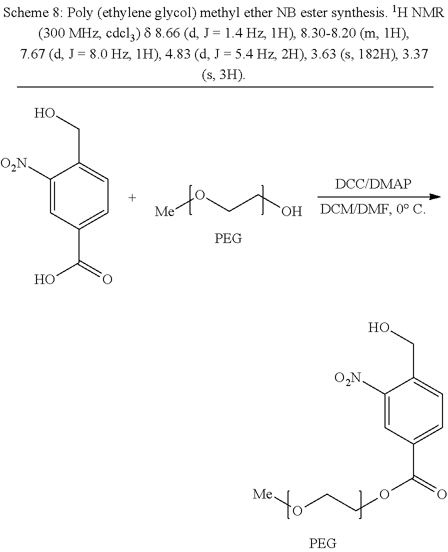

PLA ROP on o-Me PEG NB ester: Block copolymerization with lactide and glycolide was performed as described in Scheme 9, using stannous octoate as polymerization catalyst. Scheme 9: Ring opening polymerization of poly(lactic acid) on top of the free hydroxyl group. ¹H NMR (300 MHz, cdcl₃) δ 5.29-5.05 (m, 1H), 3.63 (s, 2H), 1.64-1.47 (m, 3H).

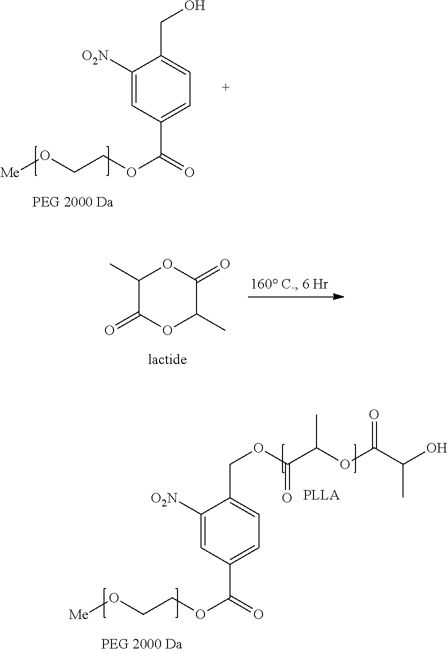

PLA ROP (ring opening polymerization) on isobutanol (Isobutene-PLA synthesis): Scheme 10: ROP on top of the free hydroxyl group of isobutanol. ¹H NMR (300 MHz, cdcl₃) δ 5.25-5.08 (m, 23H), 3.91 (dd, J=15.1, 8.6 Hz, 2H), 1.57 (t, J=8.1 Hz, 69H), 0.91 (t, J=7.7 Hz, 6H).

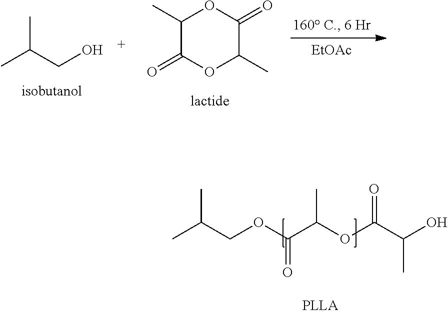

Synthesis of Isobutene-PLA Succinic Acid Ester:

Esterification reaction of PLLA with succinic anhydride: The resulting PLA with one hydroxyl end group was reacted with succinic anhydride to form PLA with one carboxyl end group that can be further conjugated (Scheme 11). “PLLA” refers to poly(L-lactic acid). In aspect, poly(D,L-lactic acid) can be used.

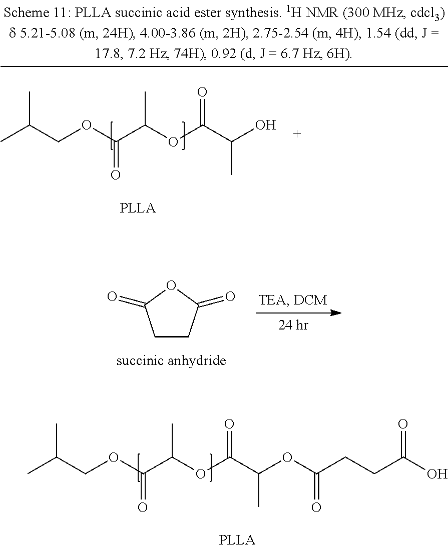

Synthesis of PEG-PLA Photo Cleavable Di-Block Co Polymer:

A coupling reaction between the carboxylic acid end group of the PLLA-succinic acid ester and the hydroxyl group of NB-PEG o-methyl ester is the final synthesis step of photo-responsive PEG-PLA, performed under moderate reaction conditions (0° C.), where the stability of the o-NB is unquestionable.

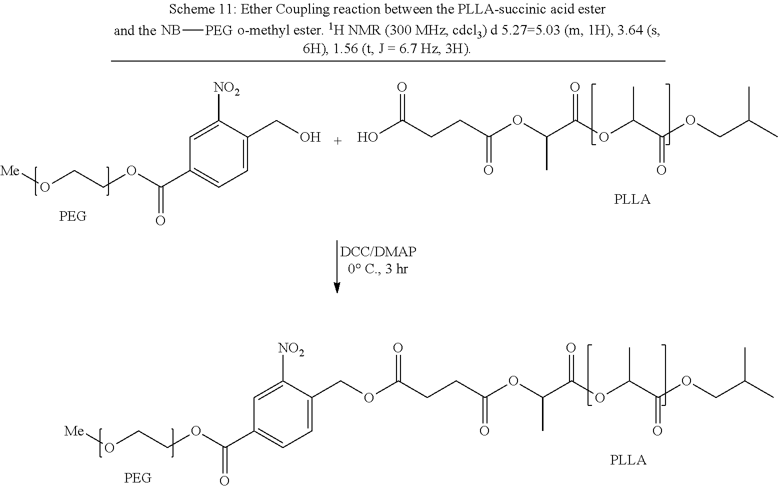

Photo Cleavable PEG-PLA Di-Block Co Polymer Molecular Weight Determination:

GPC analysis was conducted in order to observe the rise in molecular weight of the block copolymer after the coupling reaction, and ensure the coupling occurred. The GPC results shown in Table 1 demonstrate the formation of the PEG-PLA di-block copolymer, as the values of PEG-PLLA are equal to the sum of its components.

TABLE 1
Number average molecular wegith (Mn) in daltons (Da), weight
average molecular weight (Mw) in daltons (Da), and PDI values
as received from GPC analysis for o-Me-PEG-NB ester,
PLLA-succinic acid ester and PEG-PLLA di-block copolymer.

| | Mn (Da) | Mw (Da) | PDI |
|---|---|---|---|
| O-Me PEG 2000 | 3530 | 3724 | 1.05 |
| PLLA-succinic acid | 2463 | 3178 | 1.29 |
| PEG-PLLA-PS | 5286 | 6125 | 1.16 |

PEG-PLA Photo Responsive Block Copolymer Synthesis and Characterization:

A method for synthesis of PEG-PLA photo responsive block copolymer was also developed, established upon di-block of PEG and PLA connected though a junction of o-NB group, providing it with a photo-cleavable property.

PEG-PLA Photo Responsive Block Copolymer

Tri-block copolymer of PLA-PEG-PLA (Scheme 13) was prepared by using PEG diol terminated molecule, and reacting it with 2 equivalents of HM-NBA. In the next step, the product was reacted with 2 equivalents of the PLA-succinic ester, by the same procedure described above. Later, two stereo-isomers of the tri-block copolymer PLLA-PEG-PLLA and PDLA-PEG-PDLA were prepared by simply replacing the L-Lactide with D-Lactide during the ROP.

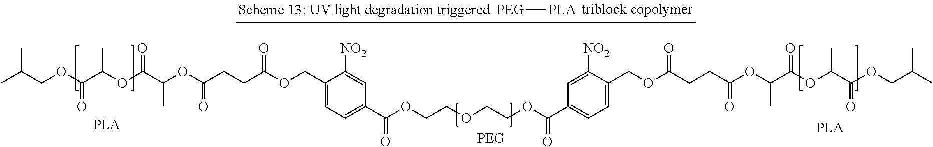

### Example 4

Synthesis of Biodegradable Thermoresponsive Collapsing Hydrogel with Triple Cleavable Bonds

Caprolactone diol 2000 (PCL, available from Sigma) was reacted with succinic anhydride in dichloromethane to form PCL docarboxylic acid. This polymer was mixed with methoxy-PEG 600 carboxylic acid and conjugated by anhydride bonds using acetic anhydride, thionyl chloride or phosgene in the presence of acid scavenger to form the anhydride bond between PCL-PEG segments. The polymer is water-soluble in iced water to form a 15% w/w solution which solidify into a gel at body temperature. The gel collapses after 7 days at 37° C. into aqueous dispersion containing PCL fragments.

### Example 5

Thermogel with S—S Cleavable Bond:

Hexadecaoxa-28,29-dithiahexapentacontanedioic acid, MW 950 (Sigma) was anhydride copolymerized with two equivalents of carboxylic acid terminated PLA-2000 or PCL1500 or poly(propylene carbonate)2000. Anhydride bond formation was affected by phosgene in chloroform solution contacting an acid scavenger. The formed polymers are soluble in iced water at 15%w/w and gels at body temperature. The gel collapses after about 7 days at 37° C. In a different experiment, an oxidizing agent, hydrogen peroxide, is added to the gel which immediately degrades the gel while the H₂O₂ is diffusing into the gel.

Alternatively, PEG diol 1000 containing an S—S bond along the PEG chain, is block copolymerized at a 1:2 w/w ratio, with lactide or lactide-glycolide 6:1 w/w ration, or caprolactone by ring opening polymerization using stannous octoate as initiator at 130oC for 24 hours. The block copolymers are soluble in iced water and immediately forms a gel when a 20% solution in water is placed in a 37° C. oven. The gel collapses when the gel is exposed to 3% hydrogen peroxide.

PLA dicarboxylic acid with oxidation cleavable disulfide bond is prepared by ring opening polymerization using hydroxyethyl disulfide (available from Sigma). The PLA disulfide-dicarboxylic acid terminated polymers of 2000 molecular weight were conjugated to PEG 600 monocarboxylic acid or mono diol to form a triblock copolymer that is soluble in iced water and immediately forms a gel when a 20% solution in water is placed in a 37° C. oven. The gel collapses when the gel is exposed to 3% hydrogen peroxide.

### Example 6

Preparation PLA-PEG-PLA with Cinnamate Light Cleavable Bonds:

p-nitrocinnamic acid was esterified to the hydroxyl end a PLA chain of an average molecular weight of 1500 using DCC as coupling agent. Similarly, PEG 1000 diol was esterified in both end with p-nitrocinnamic acid using DCC or thionyl chloride as coupling agents. A 2:1 w/w mixture of PLA-cinnamate and PEG di-cinnamate were dissolved in dichloromethane and the clear solution was irradiated at 300 nm to crosslink the cinnamate bonds and form PLA-PEG-PLA triblock gelling polymers. The solvent was evaporated to dryness and the residue was dissolved in iced cold water, the traces of polymer that did not dissolve in the water isolated and discarded. The soluble polymer was diluted in water to form a 20% w/w solution that is soluble in cold water of below 10° C. but which gels at physiological temperature. The gel collapses into a non-gel aqueous precipitate immediately after irradiation with UV lamp at 350 nm.

### Example 7

Preparation of PEG-PPG Collapsible Thermo-Gelling Polymer.

Monomethyl polyethylene glycol (PEG) 4000 carboxylic acid was added to a chloroformic solution of poly(propylene glycol) 2500 dicarboxylic acid at a 2:1 w/w ratio. To the solution, a molar equivalent amount of phosgene, or phosgene reagent, per the PPG carboxylic groups along with an acid scavenger such as triethylamine, sodium carbonate or crosslinked poly(4-vinly pyridine) resin at a 1.5 equivalents per mole phosgene. The reaction was left to conjugate overnight under dry conditions. The precipitate was isolated by filtration and the residue was concentrated and precipitated in excess hexanes. The isolated polymer is kept refrigerated in a dry bottle until use. A 20% w/w solution of the polymer was prepared in iced cold water. This solution converts into a gel when placed in a 37° C. oven. The gel become soluble when cooled in an iced bath. When the solution was placed in a 37° C. oven, the gel erodes with time by which after 10 days the gel is completely soluble in water with no gel content. FTIR analysis indicates that there are no anhydride bonds in the polymer sample after degradation while the original polymer possessed anhydride bonds at 1740 and 1800 cm⁻¹ absorption and a significant decrease in polymer molecular weight.

### Example 8

All materials were purchased from commercial sources and were used as obtained. PEG was dried by evaporating toluene to dryness. Reactions were carried out in dry glassware under inert conditions. Molecular weight was determined either by gel-permeation chromatography or nuclear magnetic resonance. Gel temperature of hydrogels was determined by the upside-down vial method.

(2-(tosyloxy)ethyl)disulfide, 2. Tosyl chloride (2.15 g, 11.3 mmol) in dichloromethane (5.6 mL) was added slowly to an ice-bath cooled mixture of 2-hydroxyethyl disulphide (575.3 mg, 3.730 mmol) and triethylamine (1.3 mL, 9.3 mmol) in dichloromethane (5.6 mL). The mixture was allowed to rise to rt and stir for 22 h, whereupon the mixture was filtered, washed 1× with 0.1 M NaOH, 2× w/DDW, dried over Na₂SO₄ and evaporated to dryness to afford 2 (1.06 g, 2.29 mmol, 61%).

PEG(SS), 3. PEG-1000 (6.25 g, 6.25 mmol) and compound 2 (1.06 g, 2.29 mmol) were melted at 80° C. overnight. The crude product was taken up in DCM and precipitated into ether to afford disulphide-containing PEG 3 (6.06 g, 83%). MW=3554 Da, Mn=2990 Da (GPC).

PLGA-PEG(SS)-PLGA, 4. Stannous Octoate (3.9 mg, 9.6 μmol) was added to a melted mixture of PEG(SS) (3, 38.98 mg, 10.97 μmol), D,L-lactide (76.4 mg, 530 μmol) and glycolide (11.24 mg, 96.8 μmol). The mixture was stirred at 120° C. for 1 h, then at 150° C. overnight. The crude polymer was taken up in DCM and precipitated into ether.

PLGA-(CO)O(CO)-PEG-(CO)O(CO)-PLGA, 6. Stannous Octoate (10.2 mg, 25.1 mol) was added to a melted mixture of PEG-600 diacid (5, 252.9 mg, 421.5 μmol), D,L-lactide (481.8 mg, 3.34 mmol) and glycolide (63.9 mg, 0.550 μmol). The mixture was stirred at 120° C. for 30 min, then at 150° C. overnight.

PLGA-PEG-PLGA triblock copolymers that contained at least one disulphide bond were synthesized. The disulphide bond causing the polymeric structure to be susceptible to degradation via reduction. A successful polymer in this case is one that dissolves at a low concentration (<30% in water), upon heating forms a gel, and the gel structure decomposes upon reduction of the disulphide bond. The synthesis of a series of disulphide bond-containing PLGA-PEG-PLGA triblock copolymers is described in Scheme 12:

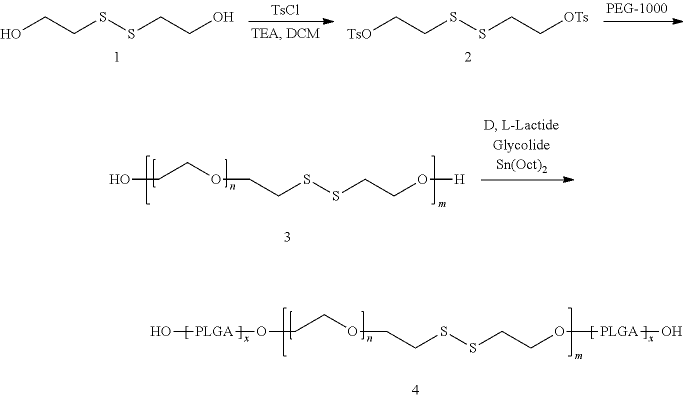

In the first step, the terminal alcohols of commercially-available 2-hydroxyethyl disulphide (1) is functionalized with tosylate groups to afford dielectrophile 2. Next, the terminal alcohols of PEG-1000 were allowed to attack the electrophilic tosylates in a melt polymerization to afford PEG with internal S—S bonds (PEG(SS), 3). The length of chain (m) was determined by GPC. Finally, ring-opening polymerization with stannous octoate catalyst and D,L-lactide and glycolide in a 6:1 molar ratio in a variety of PEG:PLGA ratios gave a series of triblock copolymer 4. The resulting polymers were purified, the L:G ratio and chain length (x) were defined, and their gelation properties in water were tested. These results are summarized in Table 2. Sample 7 provided the best result, as id displayed the gel effect.

TABLE 2

| | Molecular Weight | | |
|---|---|---|---|
| | (Mn) | L/G | |
| Sample | (PLGA-PEG-PLGA) | (NMR) | Gelation (T, % in H₂O) |
| 1 | 385-3554-385 | 7.4 | n/a |
| 2 | 621-3554-621 | 6.7 | n/a |
| 3 | 1000-3554-1000 | 5.9 | n/a |
| 4 | 1030-3554-1030 | 12.8 | n/a |
| 5 | 2692-3554-2692 | 4.6 | n/a |
| 6 | 4011-3554-4011 | 10.2 | Gel (RT, 40-60%) |
| 7 | 3882-3554-3882 | 5.3 | Gel (RT, 20%) |

A preliminary test was performed to test the sensitivity of PLGA-PEG(SS)-PLGA gel (Table 2, sample 6) to reductants. To this effect, one drop of 20 mg/mL NaBH₄ in water was added to both regular PLGA-PEG-PLGA hydrogel and to its disulphide bond-containing conjugate. No change was observed upon introduction of the reductant to the regular gel, whereas the disulphide-containing conjugate decomposed and precipitated immediately.

PLGA-PEG-PLGA triblock copolymers that contained at least one anhydride bond were synthesized. Such a hydrogel would collapse quickly in acidic or basic media based on internal anhydride bonds. Synthetic Scheme 13, wherein a PEG-diacid (5) can be used as the initiator of a PLGA-PEG-PLGA triblock copolymer (6) instead of a diol, as in 3, was followed as shown below.

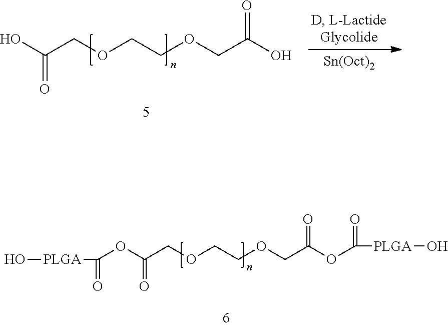

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

References: 1. Carbohydrate Polymers, 85, 2011, 261-276; 2. Molec. Pharmaceutics, 7(4) 2010, 963-968. 3. Molec. Therapy 15(6) 2007, 1189-1194; 4. Reactive & Functional Polymers (2012), 72(11), 846-855; 5. Journal of Controlled Release (2011), 152(Suppl._1), e127-e128; 6. Journal of Applied Polymer Science (2011), 122(2), 898-907; 7. Faming Zhuanli Shenqing (2009), CN 101544757 A 20090930; 8. Journal of Biomaterials Science, Polymer Edition (2009), 20(7-8), 923-934; 9. Biochimica et Biophysica Acta, Proteins and Proteomics (2007), 1774(10), 1274-1280; 10. Dehydrated scaffold seeded with cells prior to injection. Hansen K, Müller FJ, Messing M, Zeigler F, Loring J, et al. (2010) A 3-dimensional extracellular matrix as a delivery system for the transplantation of glioma-targeting neural stem/progenitor cells. Neuro-Oncology. 12(7):645-654. II. In situ polymerizable scaffold requiring mixing of two components for polymerization. Kauer T, Figueiredo J L, Hingtgen S, & Shah K. (2012) Encapsulated therapeutic stem cells implanted in the tumor resection cavity induce cell death in gliomas. Nat. Neurosci. 15(2):197-204. 12. In situ polymerizable scaffold requiring mixing of two components for polymerization. Zou Z, Denny E, Brown C E, Jensen MC, Li G, et al. (2012) Cytotoxic T Lymphocyte Trafficking and Survival in an Augmented Fibrin Matrix Carrier. PLOS ONE 7(4): e34652. doi: 10.1371/journal.pone.0034652; 13. Thermoresponsive polymers are used as a detachable culture system that can be used to generate detachable, transplantable cell sheets. Science and Technology of Advanced Materials, Volume 16, Issue 4, article id. 045003 (2015). 14. Thermoresponsive polymers (PEG-PLGA-PEG) were used to improve the engraftment of mesenchymal stem cells in diabetic ulcers. Molecular Therapy 15(6):1189-94 Jul. 2007. 15. In situ-forming chitosan/nano-hydroxyapatite/collagen gel for the delivery of bone marrow mesenchymal stem cells Carbohydrate Polymers Volume 85, Issue 1, 22 Apr. 2011, Pages 261-267; 16. In Vivo Osteogenic Differentiation of Rat Bone Marrow Stromal Cells in Thermosensitive MPEG—PCL Diblock Copolymer Gels, Tissue Eng. 2006 October; 12(10):2863-73; 17. Thermoresponsive Hydrogel as a Delivery Scaffold for Transfected Rat Mesenchymal Stem Cells Mol Pharm. 2010 Aug. 2; 7(4):963-8. doi: 10.1021/mp100094k. 18. Stimuli-responsive chitosan-starch injectable hydrogels combined with encapsulated adipose-derived stromal cells for articular cartilage regeneration, Soft Matter, 2010,6, 5184-5195. 19. Differentiation of cardiosphere-derived cells into a mature cardiac lineage using biodegradable poly(N-i sopropylacrylamide), Biomaterials. 2011 April; 32(12): 3220-32. doi: 10.1016/j.biomaterials.2011.01.050. Epub 2011 Feb. 5; 20. Hudson, W, Collins, M C, deFreitas, D, Sun, Y S, Muller-Borer, B and Kypson, A P. Beating and arrested intramyocardial injections are associated with significant mechanical loss: implications for cardiac cell transplantation. J Surg Res 142: 263-267.

1. A polymer composition comprising a non-crosslinked polymer, wherein the non-crosslinked polymer comprises a labile bond linking one or more polymer subunits.

2. The polymer composition of claim 1, comprising a plurality of non-crosslinked polymers.

3. The polymer composition of claim 1, wherein the polymer composition is a semi-solid gel at a physiological temperature, water-soluble at a temperature from about 0° C. to about 35° C., and injectable at room temperature.

4. (canceled)

5. The polymer composition of claim 1, wherein the polymer degrades upon cleavage of the labile bond.

6. (canceled)

7. The polymer composition of claim 1, wherein the polymer is a copolymer.

8. The polymer composition of claim 7, wherein the copolymer comprises a hydrophilic polymer subunit and a relatively hydrophobic polymer unit.

9. The polymer composition of claim 7, wherein the copolymer comprises:
(i) (a) ethylene glycol, 1,2-propylene glycol, 1,3-propylene glycol, 1,4-butanediol, or a combination of two or more thereof; and (b) lactic acid, glycolic acid, caprolactone, hydroxybutyric acid, hydroxyvaleric acid, hydroxycaproic acid, or a combination of two or more thereof; or
(ii) (a) ethylene glycol; and (b) 1,2-propylene glycol, 1,3-propylene glycol, 1,4-butanediol, lactic acid, glycolic acid, caprolactone, hydroxybutyric acid, hydroxyvaleric acid, hydroxycaproic acid, or a combination of two or more thereof:

10-11. (canceled)

12. The polymer composition of claim 7, wherein the copolymer comprises:
(i) (a) ethylene glycol; and (b) 1,2-propylene glycol, 1,3-propylene glycol, or a combination thereof; or
(ii) (a) ethylene glycol; and (b) lactic acid, glycolic acid, caprolactone, or a combination of two or more thereof.

13-19. (canceled)

20. The polymer composition of claim 7, wherein the copolymer is a polylactic acid-polyethylene glycol di-block copolymer, a polylactic acid-polyethylene glycol-polylactic acid tri-block copolymer, a polyethylene glycol-polylactic acid-polyethylene glycol triblock copolymer, a polyethylene glycol-polylactic acid multi-block copolymer, a polyglycolic acid-polyethylene glycol di-block copolymer, a polyglycolic acid-polyethylene glycol-polyglycolic acid tri-block copolymer, a polyethylene glycol-polyglycolic acid-polyethylene glycol triblock copolymer, a polyethylene glycol-polyglycolic acid multi-block copolymer, a poly(lactic-co-glycolic acid)-polyethylene glycol di-block copolymer, a poly(lactic-co-glycolic acid)-polyethylene glycol-poly(lactic-co-glycolic acid) tri-block copolymer, a polyethylene glycol-poly(lactic-co-glycolic acid) multi-block copolymer, a poly(1,2-propylene glycol)-polyethylene glycol di-block copolymer, a poly(1,2-propylene glycol)-polyethylene glycol-poly(1,2-propylene glycol) tri-block copolymer, a polyethylene glycol-poly(1,2-propylene glycol)-polyethylene glycol triblock copolymer, or a polyethylene glycol-poly(1,2-propylene glycol) multi-block copolymer, a poly(1,3-propylene glycol)-polyethylene glycol di-block copolymer, a poly(1,3-propylene glycol)-polyethylene glycol-poly(1,3-propylene glycol) tri-block copolymer, a polyethylene glycol-poly(1,3-propylene glycol)-polyethylene glycol triblock copolymer, a polyethylene glycol-poly(1,3-propylene glycol) multi-block copolymer, a polycaprolactone-polyethylene glycol di-block copolymer, a polycaprolactone-polyethylene glycol-polycaprolactone tri-block copolymer, a polyethylene glycol-polycaprolactone-polyethylene glycol triblock copolymer, or a polyethylene glycol-polycaprolactone multi-block copolymer.

21-25. (canceled)

26. The polymer composition of claim 1, wherein the labile bond is cleavable by hydrolysis, an enzyme, photosensitization, a pH change, a soundwave, or a combination of two or more thereof.

27. (canceled)

28. The polymer composition of claim 1, wherein the labile bond comprises an anhydride bond, a glycolic-glycolic ester bond, an imine bond, a carboxyanhydride bond, a peptide bond, an azobenzene, a triphenylmethane leucohydroxide, a nitrobenzyl, or a cinnamate.

29-40. (canceled)

41. The polymer composition of claim 7, wherein the copolymer is a copolymer of Formula (I), a copolymer of Formula (II), a copolymer of Formula (III), a copolymer of Formula (IV), or a copolymer of Formula (V): 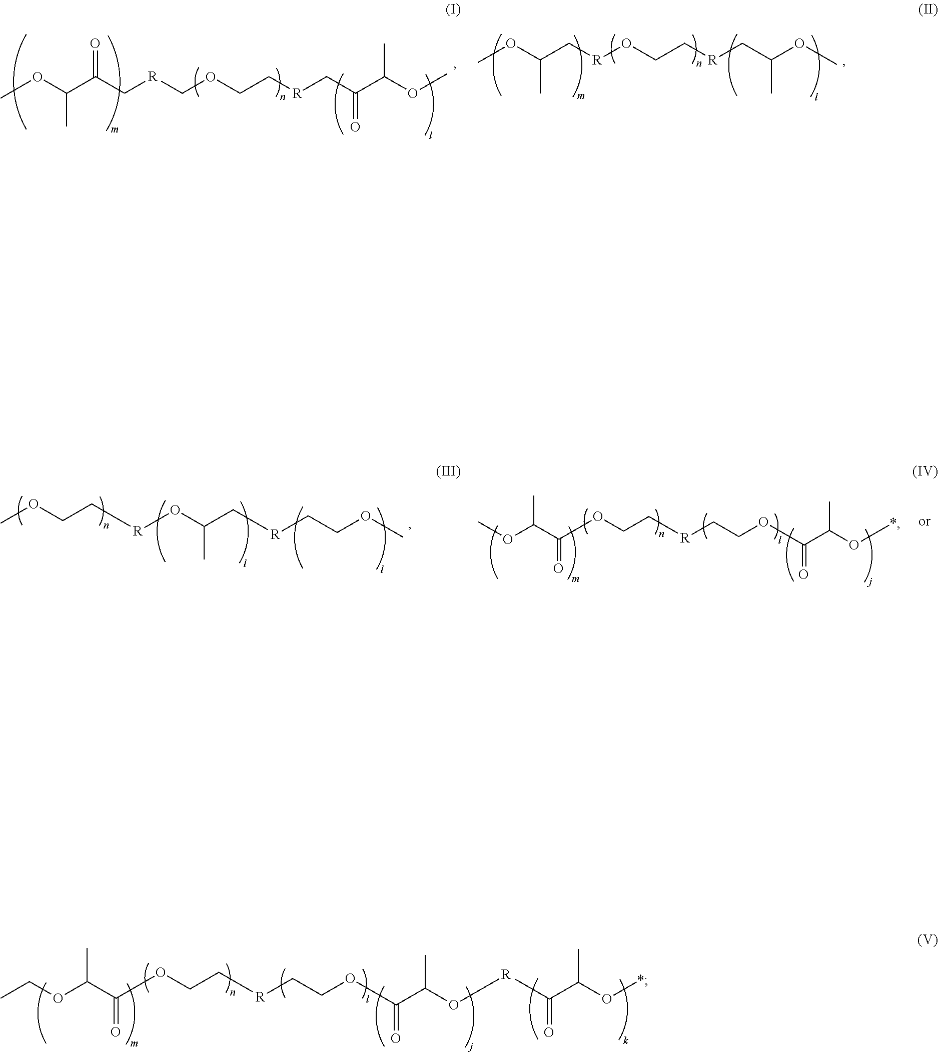
wherein m, n, l, j, and k are independently one or more polymeric subunits; * is a terminal group; and R is a labile bond.

42. The polymer of claim 41, wherein R is a labile bond cleavable by hydrolysis, an enzyme, photosensitization, a pH change, a soundwave, or a combination of two or more thereof.

43. The polymer of claim 41, wherein R is independently —C(═O)—O—C(═O)—, —N═CH—, —C(═O)—CH₂—O—C(═O)-, or —S—S—.

44. (canceled)

45. The polymer of claim 41, wherein R is a labile bond comprising an anhydride bond, a glycolic-glycolic ester bond, an imine bond, a carboxyanhydride bond, a peptide bond, an azobenzene, a triphenylmethane leucohydroxide, a nitrobenzyl, or a cinnamate.

46-57. (canceled)

58. A pharmaceutical composition comprising the polymer composition of claim 1, a therapeutic agent, and a pharmaceutically acceptable excipient.

59. The pharmaceutical composition of claim 58, wherein the therapeutic agent is an antibody, a functional fragment of an antibody, a small molecule chemical compound, a nucleic acid, a polypeptide, a contrasting agent, a cell, or a combination of two or more thereof.

60-61. (canceled)

62. A method for delivering a therapeutic agent to a subject in need thereof, the method comprising administering to the subject the pharmaceutical composition of claim 1.

63-65. (canceled)

66. A kit comprising the polymer composition of claim 1 and written instructions for administering the polymer composition to a subject.

67-70. (canceled)

71. A syringe, a catheter, or a bio-ink comprising the polymer composition of claim 1.

72-77. (canceled)
